(19) United States
(12) Patent Application Publication
(10) Pub. No.: US 2026/0158161 A1
(43) Pub. Date: Jun. 11, 2026

(54) AN ADJUVANT AND CD47-BLOCKADE COMBINATION CANCER TREATMENT AND METHODS OF USING THE SAME

(71) Applicant: BOARD OF REGENTS, THE UNIVERSITY OF TEXAS SYSTEM, Austin, TX (US)

(72) Inventors: Yon Son Betty KIM, Houston, TX (US); Wen JIANG, Houston, TX (US); Yifei LU, Houston, TX (US); Kristin HUNTOON, Houston, TX (US)

(73) Assignee: BOARD OF REGENTS, THE UNIVERSITY OF TEXAS SYSTEM, Austin, TX (US)

(21) Appl. No.: 19/127,045

(22) PCT Filed: Nov. 2, 2023
(86) PCT No.: PCT/US2023/078492

§ 371 Date: May 2, 2025

Related U.S. Application Data
(60) Provisional application No. 63/382,251, filed on Nov. 3, 2022.

(51) Int. Cl. A61K 47/68; A61K 39/395; A61P 35/00

(52) U.S. Cl. CPC A61K 47/6849; A61K 39/3955; A61K 47/6811; A61K 47/6883; A61P 35/00

(57) ABSTRACT

Provided are materials and methods for adjuvant and CD47 blockage cancer therapy. The adjuvant can be a nanoparticle with a cancer binding molecule and a SLAMF7 polypeptide attached. The binding of the nanoparticle to the cancer cell through the cancer binding molecule decorates the cancer cell with SLAMF7 polypeptides and allows the binding of an antigen presenting cell to the cancer cell and phagocytosis of the cancer cell by the antigen presenting cell.

### CROSS-REFERENCE TO RELATED APPLICATIONS

This application claims the benefit of U.S. Provisional Application No. 63/382,251, filed Nov. 3, 2022, which is incorporated by reference herein in its entirety.

### GOVERNMENT FUNDING STATEMENT

This invention was made with government support under W81XWH-19-1-0325 awarded by the Medical Research and Materiel Command, and CA241070 awarded by the National Institutes of Health. The government has certain rights in the invention.

### REFERENCE TO SEQUENCE LISTING SUBMITTED ELECTRONICALLY

The content of the electronically submitted sequence listing in ASCII text file (Name 4443_032PC01_Seqlisting_ST26; Size: 18,289 bytes; and Date of Creation: Oct. 26, 2023) filed with the application is incorporated herein by reference in its entirety.

### BACKGROUND

Solid tumors display limited response to immunotherapies. By contrast, hematological malignancies exhibit significantly higher response rates to immunotherapies as compared to solid tumors. Although the reason for this disparity in cancer immunotherapy response is multifactorial, one possible contributing factor is the distinctive expression patterns of surface proteins on hematologic and solid tumour cells that regulate immune cell functions. One membrane bound protein, signaling lymphocytic activation molecule family member 7 (SLAMF7), was recently reported to be exclusively expressed by hematopoietic cells including cancer cells, but not in cells from solid tumour (Chen et al., Nature, 544:493-497 (2017). SLAMF7 was discovered to be a “pro-phagocytosis” molecule expressed by liquid tumors, but not by solid tumors (Chen et al., Nature, 544:493-497 (2017)). Studies also demonstrated that without the presence of SLAMF7, the innate immune system (first responders of immunity) cannot detect and engulf solid tumors, and thus allows solid tumors to grow and spread (Chen et al, Nature, 544:493-497 (2017)). The exclusive expression of SLAMF7 by hematologic cancers was postulated to govern their responsiveness to cancer immunotherapy such as CD47 blockade (Uger, R. & Johnson, L. Blockade of the CD47-SIRPalpha axis: a promising approach for cancer immunotherapy. Expert Opin. Biol. Ther. 20, 5-8 (2020); Zhong, C. et al. Poly(I:C) enhances the efficacy of phagocytosis checkpoint blockade immunotherapy by inducing IL-6 production. J. Leukoc. Biol. (2021); Cao, X. et al. Effect of cabazitaxel on macrophages improves CD47-targeted 363 immunotherapy for triple-negative breast cancer. J. Immunother. Cancer 9 (2021); Zhang, A. L. et al. Dual targeting of CTLA-4 and CD47 on T-reg cells promotes immunity against solid tumors. Sci. Transl. Med. 13 (2021)).

### BRIEF SUMMARY OF THE INVENTION

Provided is a particle comprising a nanoparticle, a first molecule which binds a tumor cell, and a second molecule which allows the tumor cell to be recognized by an antigen presenting cell, wherein the first and the second molecules are linked to the nanoparticle with a molecular ratio of about 1:10 to about 10:1.

In some aspects, the first molecule and the second molecule are covalently linked to the nanoparticle.

In some aspects, the first molecule is selected from an antibody, a single-chain antibody, a nanobody, and a non-antibody antigen-binding polypeptide.

In some aspects, the first molecule binds to a protein expressed on the tumor cell selected from the group consisting of HER2, EGFR, EGFRviii, PSMA, CEA, CA125, TROP2, CD105, GD2, GD3, VEGFR1, VEGFR2, NCAM, CD133, ADAM17, MCSP, PSCA, FOLR1, EPCAM, HER3, EGFR, PSMA, Integrin α4β1, FAP, MUC-1, P glycoprotein, AFP, ALK, CD30, ErbB3, ErbB4, Mesothelin, CAIX, CD66e, cMet, EphA2, HGF/SF, TPBG, BRCA1, BORIS, FOLR1, GAGE-1, GAGE-2, GM3, gp100, MAGE-A3, MAGE1, MAGE2, MAGE3, MAGE4, MAGE6, MAGE12, MART-1, Muc1, Muc3A, Muc3B, Muc4, Muc12, Muc13, Muc15, Muc16, Muc17, Muc21, Muc22, PRLR, or CD340.

In some aspects, the first molecule binds to a HER2 protein or EGFR protein expressed on the tumor cell.

In some aspects, the first molecule comprises an antibody or antigen-binding fragment that binds a HER2 or an EGFR.

In some aspects, the first molecule is selected from the group consisting of folate, mannose, dehydroabietic acid, and any combination thereof.

In some aspects, the second molecule is not an antibody.

In some aspects, the second molecule comprises a signaling lymphocytic activation molecule family member 7 (SLAMF7) polypeptide or a functional fragment thereof.

In some aspects, the second molecule comprises a full-length SLAMF7 polypeptide.

In some aspects, the second molecule comprises an extracellular domain of SLAMF7.

In some aspects, the second molecule comprises an Ig-like domain of SLAMF7.

In some aspects, the second molecule comprises an Ig-like V-type domain of SLAMF7.

In some aspects, the second molecule comprises an Ig-like C2-type domain of SLAMF7.

In some aspects, the SLAMF7 is a human SLAMF7 polypeptide or a functional fragment thereof.

In some aspects, the molecular ratio of the first molecule to the second molecule is about 1:3 to 3:1.

In some aspects, the molecular ratio of the first molecule to the second molecule is about 1:3.

In some aspects, the nanoparticle comprises a poly lactic acid polymer, a polyglycolic acid polymer, a poly(D,L-lactic-co-glycolic acid) co-polymer, a polyethylene glycol polymer, a poly(D,L-lactic-co-glycolic acid)-b-poly(ethylene glycol) copolymer with terminal methoxy groups (PLGA-b-mPEG), a poly(D,L-lactic-co-glycolic acid)-b-poly(ethylene glycol) copolymer with terminal N-hydroxysuccinimide ester groups (PLGA-b-PEG-NHS), a poly(D,L-lactic-co-glycolic acid)-b-poly(ethylene glycol) copolymer with terminal folic acid groups (PLGA-b-PEG-Folate), or any combination thereof.

In some aspects, the nanoparticle has a size of about 70 nm to about 85 nm.

In some aspects, the nanoparticle has a polydispersity index of about 0.09 to about 0.4.

In some aspects, the nanoparticle is stable for at least 72 hours at 37° C.

In some aspects, the first molecule comprises a HER2 antibody or antigen-binding fragment thereof and the second molecule comprises full-length SLAMF7 polypeptide.

In some aspects, the particle comprises about 100 to 500 first molecules (e.g., HER2 antibody or antigen-binding fragments thereof molecules) and about 500 to 1800 second molecules (e.g., full length SLAMF7 polypeptide molecules).

In some aspects, the particle comprises about 120 to 400 first molecules (e.g., HER2 antibody or antigen-binding fragments thereof molecules) and about 500 to 1600 second molecules (e.g., full length SLAMF7 polypeptide molecules).

In some aspects, the particle comprises about 144 and 360 HER2 antibody or antigen-binding fragments thereof molecules and about 522 and 1564 full length SLAMF7 polypeptide molecules.

In some aspects, the particle comprises 144 HER2 antibody or antigen-binding fragments thereof molecules and 1564 full length SLAMF7 polypeptide molecules.

In some aspects, provided is a composition comprising a particle as described herein and a pharmaceutically acceptable carrier.

In some aspects, also provided is a combination therapy comprising a particle as described herein, a composition as described herein and a CD47 blocking agent.

In some aspects, the CD47 blocking agent used in the combination therapy is selected from the group consisting of an anti-CD47 antibody or antigen-binding fragment thereof, an anti-CD47 nanobody, an anti-CD47 single chain antibody, a CD47 binding polypeptide, and SIRPalpha inhibitor.

In some aspects, the CD47 blocking agent used in the combination therapy comprises an anti-CD47 antibody or antigen-binding fragment thereof.

In some aspects, the CD47 blocking agent used in the combination therapy comprises a SIRPalpha inhibitor.

In some aspects, the combination therapy further comprises an immune checkpoint inhibitor, which is not the CD47 blocking agent.

In some aspects, the immune checkpoint inhibitor is selected from the group consisting of a PD-1 antibody, a fragment thereof, a PD-1 single chain antibody, a PD-1 nanobody, a PD-1 binding polypeptide, a PD-L1 antibody, a fragment thereof, a PD-L1 single chain antibody, a PD-L1 nanobody, a PD-L1 binding polypeptide, and any combination thereof.

In some aspects, the immune checkpoint inhibitor is selected from a PD-1 blocking agent, a PD-L1 blocking agent, or a combination thereof.

In some aspects, provided is a method of treating a cancer in a subject comprising administering to the subject a therapeutically effective amount of the particle as described herein, the composition as described herein, or the combination therapy as described herein.

In some aspects, the cancer treated is a solid tumor.

In some aspects, the cancer is a breast cancer, a brain cancer, a prostate cancer, a lung cancer, a colorectal cancer, a skin cancer, a head and neck cancer, or a pancreatic cancer.

In some aspects, the particle and the CD47 blocking agent are administered at the same time or sequentially.

In some aspects, the particle, the CD47 blocking agent, and the immune checkpoint inhibitor are administered at the same time or sequentially.

In some aspects, the sequential administration comprises administration of the particle, the CD47 blocking agent, and/or the immune checkpoint inhibitor consecutively or administration together in one or more compositions consecutively.

In some aspects, the particle is administered via a route selected from intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrasternal, oral, rectal, topical, epidermal, mucosal, intranasal, vaginal, rectal, sublingual administration, and combinations thereof.

In some aspects, the CD47 blocking agent is administered via a route selected from intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrasternal, oral, rectal, topical, epidermal, mucosal, intranasal, vaginal, rectal, sublingual administration, and combinations thereof.

In some aspects, the immune checkpoint inhibitor is administered via a route selected from intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrasternal, oral, rectal, topical, epidermal, mucosal, intranasal, vaginal, rectal, sublingual administration, and combinations thereof.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS/FIGURES

FIGS. 1A-1C. FIG. 1A shows size, zeta potential, and polydispersity index of nanoparticles (NPs) alone, NPs with attached HER2 antibodies, NPs with attached SLAMF7 polypeptide and NPS with attached HER2 antibodies and SLAMF7 polypeptide (BiTNHER). FIG. 1B shows valency of BiTNHER NPs. The left column indicates the molecular ratio of anti-HER2 antibody and SLAMF7 added into the conjugation reaction; the right two columns indicates the number of copies of anti-HER2 antibody and SLAMF7 per nanoparticle. FIG. 1C shows specific binding of HER-NPs with the HER2/neu high mouse breast cancer cell lines EO771/E2 and TUBO, compared with HER2/neu low 71 EO771 and 4T1 cells. Scale bar, 50 μm.

FIGS. 2A-2C. FIG. 2A shows nanoparticle size in PBS or FBS-containing media within 72 hours. FIG. 2B shows polydispersity index (PDI) of nanoparticles in PBS or FBS-containing media within 72 hours. FIG. 2C shows loaded protein of nanoparticle in PBS or FBS-containing media within 72 hours.

FIGS. 3A-3C. FIG. 3A shows expression of SLAMF7 on the hematopoietic mouse leukemia cell line L1210, mouse bone marrow-derived macrophage (BMDMs), mouse tumor-associated macrophages (TAM), and the human macrophage cell line THP-1. FIG. 3B shows flow cytometry images of anti-CD47 antibody-mediated macrophage phagocytosis of SLAMF7 high L1210 cells compared to SLAMF7 negative breast cancer cell lines EO771 or EO771/E2. FIG. 3C shows a bar graph of the flow cytometry results of FIG. 3B.

FIGS. 4A-4F. FIG. 4A shows a schematic of the nanoparticle-based conversion strategy via anti-HER2 antibody targeting and SLAMF7 labeling of HER2-expressing cancer cells. FIG. 4B shows the size distribution of unconjugated PEG-PLGA nanoparticles (NPs) and BiTNHER measured by dynamic light scattering. FIG. 4C shows a gel electrophoresis of unconjugated NP and NP conjugated with anti-HER2 antibody (HER-NP), SLAMF7 (S-NP), and both (BiTNHER). FIG. 4D shows confocal images and a FACS semiquantitative analysis of HER-NP labeling of the HER2-high expressing human breast cancer cell line SK-BR-3 and the HER2-low expressing cell line MDA-MB-468; green, NPs; blue, DAPI; scale bar, 50 μm. FIG. 4E shows confocal images and a FACS semiquantitative analysis of HER2-low MDA-MB-468 cells and HER2-high SK-BR-3 cells after incubation with IgG-SLAMF7-NP or HER-SLAMF7-NP and cell staining with anti-SLAMF7 antibody and anti-human IgG antibody; scale bar, 50 μm, higher-magnification image of SK-BR-3 cells in the outlined area, scale bar, 20 μm. FIG. 4F shows binding affinity of nanoparticles with different HER:SLAMF7 conjugation ratios to HER2-expressing SK-BR-3 cells.

FIGS. 5A-5D. FIG. 5A shows a western blotting confirming the knock-down of SLAMF7 in THP-1 via siRNA silencing. FIG. 5B shows macrophage phagocytosis of THP1 and SLAMF7 knock-down THP1 of SK-BR-3 cancer cells. FIG. 5C shows a western blotting confirming the overexpression of SLAMF7 on SK-BR-3 9 and MDA-MB-468 via plasmid transfection. FIG. 5D shows enhanced macrophage phagocytosis of SK-BR-3/SLAMF7+ and MDA-MB-468/SLAMF7+ in the presence of CD47 blockade.

FIG. 6 shows confocal imaging using a pHrodo assay confirming BiTNHER induced phagocytosis of EO771/E2 cancer cells (blue) colocalized with pHrodo bioparticles (green) in same macrophages (arrow heads). Scale bar, 20 μm.

FIG. 7 shows BiTNHER (with aCD47) induced macrophage phagocytosis in two different cancer cell lines.

FIGS. 8A-8B. FIG. 8A shows macrophage phagocytosis of HER2-positive EO771/E2 cells with increasing concentrations of BiTNHER. FIG. 8B shows macrophage phagocytosis of HER2-positive EO771/E2 cells in the presence of 0.4 mg/ml BiTNHER2 and different amounts of aCD47.

FIGS. 9A-9B. FIG. 9A shows SLAMF7 expression on macrophages polarized to M1 by lipopolysaccharide and to M2 with IL-4. FIG. 9B shows macrophage phagocytosis of HER2-high EO771/E2 cells of M1 polarize dnad M2 polarized macrophages in the presence of the combination treatment of BiTNHER and aCD47.

FIGS. 10A-10C. FIG. 10A shows a SDS-PAGE analysis of Fc fragment digestion and F(ab′)2 generation from anti-HER2 antibody. Red line indicated the band of F(ab′)2 around 110 kDa. FIG. 10B shows that a purified F(ab′)2 fragment of anti-HER2 antibody had similar binding affinity to HER2-overexpressing EO771/E2 cells as the anti-HER2 IgG. FIG. 10C shows BMDM phagocytosis of HER2-low EO771 and HER2-high EO771/E2 mouse breast cancer cells in the presence of aCD47 after treatment with PBS, NP with unconjugated anti-HER2 antibody (IgG or F(ab′)2 fragment) and SLAMF7, or BiTNHER conjugated with anti-HER2 antibody (IgG or F(ab′)2 fragment).

FIGS. 11A-11E. FIG. 11A shows IVIS imaging of mice after i.t. injection of BODIPY-loaded nanoparticles. FIG. 11B shows tumor signal decline over time after i.t. injection. FIG. 11C shows ex vivo imaging of major organs 24 hours after i.t. injection. FIG. 11D shows a quantitive measurement of NP signals in major organs 24 hours after i.t. injection. FIG. 11E shows confocal imaging of intratumoral distribution of BODIPY-labeled nanoparticles. Red, BODIPY; Blue, DAPI. Scale bar, 50 μm.

FIGS. 12A-12B. FIG. 12A shows tumor size of TUBO tumor-bearing BALB/c mice treated with different amounts of aCD47 and treated with 4 mg/kg BiTNHER in combination with different amounts of aCD47. FIG. 12B shows survival of TUBO tumor-bearing BALB/c mice treated with aCD47 at different amounts and treated with 4 mg/kg BiTNHER in combination with different amounts of aCD47.

FIGS. 13A-13I. FIG. 13A shows human THP-1 phagocytosis of HER2-expressing SK-BR-3 cancer cells in the presence of aCD47 and BiTN_{HER} with different SLAMF7:HER conjugation ratios. FIG. 13B shows BiTN_{HER} converted HER2/neu-expressing human (SK-BR-3) and mouse (EO771/E2) breast cancer cells into SLAMF7-high cells and promoted human THP-1 or mouse (C57BL6 bone marrow) macrophage phagocytosis in the presence of aCD47, comparable with the SLAMF7-expressing mouse leukemia L1210 cells. FIG. 13C shows that anti-SLAMF7 antibody abrogated the pro-phagocytosis effect of BiTN_{HER} and aCD47 on HER2-expressing cancer cells. FIG. 13D shows phagocytosis of CFSE-labelled HER2-low EO771 and HER2-high EO771/E2 mouse breast cancer cells and SLAMF7-high L1210 mouse leukemia cells by mouse bone marrow macrophages in the presence of aCD47 after treatment with NP alone, NP with unconjugated anti-HER2 antibody and SLAMF7, or BiTN_{HER}. Red, macrophages; green, cancer cells; sscale bar, 50 μm. FIG. 13E shows BiTN_{HER} with aCD47 promotes macrophage phagocytosis against HER2-expressing breast cancer cells. FIG. 13F shows increased antigen presentation of the H2kb-SIINFEKL complex on macrophages after phagocytosis of BiTN_{HER}-treated HER2-expressing EO771/E2-cOVA cells. Green, macrophages; red, H2kb-SIINFEKL complex; scale bar, 50 μm. FIG. 13G shows increased macrophage antigen presentation of HER2-expressing cancer cells in the presence of a combination of BiTN_{HER} and aCD47. FIG. 13H shows proliferation and percentage of total CD8+ cells of cOVA antigen-specific OT-I T cells in the presence of a combination of BiTN_{HER} and aCD47. FIG. 13I shows proliferation and percentage of total CD4+ cells of cOVA antigen-specific OT-II T cells in the presence of a combination of BiTN_{HER} and aCD47.

FIGS. 14A-14H. FIG. 14A shows tumors and tumor sizes of HER2-high TUBO breast cancer cells implanted in BALB/c mice following treatment with a combination of BiTNHER and aCD47 and effects on the survival of the mice. FIG. 14B shows tumors and tumor sizes of HER2-low 4T1 breast cancer cells implanted in BALB/c mice following treatment with a combination of BiTNHER and aCD47 and effects on the survival of the mice. FIG. 14C shows that combined treatment with BiTNHER and aCD47 induced the greatest tumor-inhibition effect in HER2/neu expressing TUBO tumors, with 3 of 6 mice remaining tumor-free (TF). FIG. 14D shows that combined treatment with BiTNHER and aCD47 promoted the infiltration of dendritic cells (DCs) and macrophages within TUBO tumors relative to 4T1 tumors. FIG. 14E shows that combined treatment with BiTNHER and aCD47 increased the infiltration of CD4+T and CD8+ T cells into TUBO tumors. FIG. 14F shows that combined treatment with BiTNHER and aCD47 increased the number of IFNγ-producing CD8+ T cells in TUBO tumors. FIG. 14G shows that combined treatment with BiTNHER and aCD47 and decreased the number of intratumoral regulatory CD4+ T cells. FIG. 14H shows that combined treatment with BiTNHER and aCD47 increased the peripheral-blood cytokine levels in TUBO tumor-bearing mice but not in 4T1 tumor-bearing mice.

FIG. 15 shows infiltration of NK cells within TUBO tumors compared with 4T1 tumors in the presence of combined treatment with BiTNHER and aCD47.

FIGS. 16A-16B. FIG. 16A shows intratumoral F4/80+ macrophages and Iba-1+ activated macrophages in HER2-high TUBO breast tumors in the presence of combined treatment with BiTNHER and aCD47. FIG. 16B shows intratumoral F4/80+ macrophages and Iba-1+ activated macrophFages in HER2-low 4T1 breast tumors in the presence of combined treatment with BiTNHER and aCD47.

FIGS. 17A-17B. FIG. 17A shows CD4+ and CD8+ lymphocyte infiltration in HER2-high TUBO breast tumors in BALB/c mice in the presence of combined treatment with BiTNHER and aCD4. FIG. 17B shows CD4+ and CD8+ lymphocyte infiltration in HER2-low 4T1 breast tumors in BALB/c mice in the presence of combined treatment with BiTNHER and aCD47.

FIGS. 18A-18E. FIG. 18A shows the in vivo experimental design for CD8 depletion in TUBO tumor bearing mice. FIG. 18B shows a flow cytometry analysis of CD8 expression on splenocytes after aCD8 treatment. FIG. 18C shows tumor size in TUBO tumor bearing mice treated with combination treatment of BiTNHER and aCD47 in the absence and presence of aCD8. FIG. 18D shows survival of TUBO tumor bearing mice treated with combination treatment of BiTNHER and aCD47 in the absence and presence of aCD8. FIG. 18E shows that CD8+ T cell depletion significantly decreased the infiltration of CD4+ cells, CD8+ T cells, and dendritic cells (DCs), especially the number of infiltrated IFNγ-producing CD8+ T cells, while increasing the number of immunosuppressive myeloid-derived suppressor cells (MDSCs).

FIGS. 19A-19B. FIG. 19A shows flow cytometric analysis of splenocytes from TUBO tumor-bearing mice showing that the combination of BiTNHER and aCD47 induced a shift in naïve CD4+ and CD8+ T cells towards memory phenotypes. FIG. 19B shows flow cytometric analysis of splenocytes from TUBO tumor-bearing mice showing that the combination of BiTNHER and aCD47 induced a shift in naïve CD4+ and CD8+ T cells towards central memory phenotypes but not effective memory phenotypes.

FIGS. 20A-20F. FIG. 20A shows that tumor growth was inhibited by the triple-combination of BiTNHER, aCD47, and aPD1 treatment and significantly prolonged the survival of EO771/E2 tumor-bearing mice. FIG. 20B shows that STING knockout partially abrogated the antitumor effect of the triple-combination treatment in EO771/E2-bearing mice. FIG. 20C shows immune-fluorescence staining of EO771/E2 tumors implanted in wild-type (WT) mice. FIG. 20D shows immune-fluorescence staining of EO771/E2 tumors implanted in STING knockout mice. Scale bar, 50 μm. Areas within the dashed squares are shown at higher magnification in the images to the right; arrows indicate the nuclear-translocated pIRF3 within F4/80+ macrophage or CD11c+ dendritic cells. Scale bar, 20 μm. FIG. 20E shows the quantification of infiltrated F4/80+ macrophages and percentage of nuclear pIRF3+ macrophages in the tumors. FIG. 20F shows that the expression of type I interferons is elevated in intratumoral F4/80+ macrophages after triple combination treatment with BiTNHER, aCD47, and aPD1. IFNA, interferon α; IFNB, interferon β.

FIGS. 21A-21B. FIG. 21A shows no significant change of complete blood count was observed on mice 30 days after treatment. Healthy, healthy Balb/c mice without treatment; Healthy Treated, healthy Balb/c mice with subcutaneous injection of BiTNHER and aCD47; TUBO Treated, TUBO-bearing Balb/c mice with intratumoral injection of BiTNHER and aCD47. WBC, white blood cell; RBC, red blood cell; MCH, mean corpuscular hemoglobin; MCV, mean corpuscular volume; MCHC, mean corpuscular hemoglobin concentration; HGB, hemoglobin; HCT, hematocrit; SEGS, segmental neutrophils; MONOS, monocytes; EOS, eosinophils; BASOS, basophils; LYMPHS, lymphocytes; LUC, large unstained cells. FIG. 21B shows H&E staining of major organs 30 days after the 195 last treatment. No obvious inflammation or lymphocyte infiltration was observed on mice 30 days after treatment. Healthy, healthy Balb/c mice without treatment; Healthy Treated, healthy Balb/c mice with subcutaneous injection of BiTNHER and aCD47; TUBO Treated, TUBO-bearing Balb/c mice with intratumoral injection of BiTNHER and aCD47.

FIGS. 22A-22H. FIG. 22A shows tumor growth in EO771/E2-bearing mice treated with BiTNHER and aCD47. FIG. 22B shows survival of EO771/E2-bearing mice. FIG. 22C shows immunofluorescence staining of PD1 and PDL1 expression in the relatively unresponsive tumor type EO771/E2 compared with responding tumor type TUBO. Scale bar, 50 μm. FIG. 22D shows tumors in EO771-bearing mice treated with the triple combination treatment of BiTNHER, aDC47, and aPD1. FIG. 22E shows tumor infiltrating CD4+ and CD8+ cells in EO771-bearing mice treated with the triple combination treatment of BiTNHER, aDC47, and aPD1. FIG. 22F shows tumor infiltrating FOXP3+ and IFNγ+ CD4+ and CD8+ T cells in EO771-bearing mice treated with the triple combination treatment of BiTNHER, aDC47, and aPD1. FIG. 22G shows tumor infiltrating dendritic cells and macrophages in EO771-bearing mice treated with the triple combination treatment of BiTNHER, aDC47, and aPD1. FIG. 22H shows cytokine levels in EO771-bearing mice treated with the triple combination treatment of BiTNHER, aDC47, and aPD1.

FIGS. 23A-23B. FIG. 23A shows immunofluorescent images of CD4+ and CD8+ cell infiltration in EO771/E2 and EO771 tumors from C57BL/6 mice treated with the triple combination treatment (BiTNHER+aCD47+aPD1). FIG. 23B shows immunofluorescent images of macrophages and activated macrophages in EO771/E2 and EO771 tumors from C57BL/6 mice treated with the triple combination treatment (BiTNHER+aCD47+aPD1).

FIG. 24 shows tumor size in EO771/E2 tumor-bearing mice treated with the combination treatment of BiTNHER and aPD1 or BiTNHER, aCD47 and aPD1.

FIGS. 25A-25B. FIG. 25A shows fluorescent images of CD4+ and CD8+ cells in EO771/E2 and EO771 tumors from STING knockout mice treated with aCD47+aPD1 or BiTNHER, aCD47 and aPD1. FIG. 25B shows fluorescent images of macrophages and activated macrophages in EO771/E2 and EO771 tumors from STING knockout mice treated with aCD47+aPD1 or BiTNHER, aCD47 and aPD1.

FIGS. 26A-26D. FIG. 26A shows immunofluorescent images of EO771/E2 tumors implanted in WT mice. FIG. 26B shows immunofluorescent images of EO771/E2 tumors implanted in STING knockout mice. Areas within the dashed squares are shown at higher magnification to the right; arrowheads indicate the nuclear translocation of pIRF3 within CD11c+ dendritic cells; scale bar, 50 μm. FIG. 26C Quantification of infiltrated CD11c+ DCs and percentage of nuclear pIRF3+ DCs in the tumors. FIG. 26D Expression of type I interferons in intratumoral CD11c+ macrophages following the triple combination therapy BiTNHER, aCD47 and aPD1.

FIGS. 27A-27H. FIG. 27A shows the experimental design for establishing 4T1 metastasis model and two treatment strategy. Treatment A, intratumoral pretreatment of primary tumor, followed by additional i.v. injection of BiTNFo after surgery; Treatment B, direct i.v. injection of BiTNFo after surgery without pretreatment. FIG. 27B shows that the triple combination treatment inhibited the growth of 4T1 primary tumors. FIG. 27C shows that the triple combination treatment increased the infiltration of IFNγ+ cytotoxic CD8+ T cells and decreased the infiltration of immunosuppressive CD4+ Treg cells. FIG. 27D shows that the triple combination treatment increased the infiltration of dendritic cells (DCs) and decreased the infiltration of myeloid-derived suppressor cells (MDSCs). FIG. 27E shows IVIS imaging (left) and quantification of whole-body signal (right) showing decreased number and size of tumor metastases after triple combination treatment. FIG. 27F shows that combination treatment induced long-term protection against tumor metastasis, with 2 of 7 surviving mice remaining tumor-free. FIG. 27G shows IVIS imaging (left) and quantification of whole-body signal (right). FIG. 27H shows that combination treatment induced long-term protection against tumor metastasis.

FIGS. 28A-28D. FIG. 28A shows folate receptor (FR) a expression in triple-negative 4T1 breast cancer cells and TUBO cells. FIG. 28B shows the size distribution of folate-conjugated nanoparticles (Folate-NP) and folate/SLAMF7-conjugated nanoparticles (BiTNFo) measured by diffuse light scattering. FIG. 28C shows phagocytosis of BiTNFo at different folate:SLAMF7 conjugation ratios in FR-high 4T1 cancer cells. FIG. 28D shows phagocytosis of FR-high 4T1 cancer cells in the presence of different nanoparticles in the absence and presence of aCD47.

FIGS. 29A-29H. FIG. 29A shows the experimental design for establishing a 4T1 tumor metastasis model and treatment. FIG. 29B shows tumor size in 4T1-bearing BALB/c mice treated with the BiTNFo+aCD47 combination treatment. FIG. 29C shows tumor infiltrating T cells in 4T1-bearing BALB/c mice. FIG. 29D shows tumor infiltrating dendritic cells and macrophages in 4T1-bearing BALB/c mice. FIG. 29E shows flow cytometric analysis of splenocytes from 4T1 tumor-bearing mice showing that BiTNFo+aCD47 induced a shift in naïve CD4+ and CD8+ T cells towards memory phenotypes. FIG. 29F shows IVIS imaging of tumor metastases after surgical removal of the primary tumor in mic treated with a BiTNFo+aCD47 combination treatment at a low and high dose. FIG. 29G shows tumor size and survival after the 3rd treatment with BiTNFo+aCD47 for good responders vs poor responders. FIG. 29H shows fluorescent images of PD1 and PDL1 levels in the tumor microenvironment of good responders vs poor responders to the BiTNFo+aCD47 combination treatment.

FIGS. 30A-30D. FIG. 30A shows the biocompatibility studies for BiTNFo demonstrating no obvious hemolysis after intravenous injection of BiTNFo at 40 mg/kg. FIG. 30B shows no significant change in body weight after intravenous injection of BiTNFo at 40 mg/kg. FIG. 30C shows no significantly change in the peripheral blood levels of urea nitrogen (BUN), aspartate aminotransferase (AST), alanine aminotransferase (ALT) after intravenous injection of BiTNFo at 40 mg/kg. FIG. 30D shows no toxicity to major organs after intravenous injection of BiTNFo at 40 mg/kg.

FIG. 31 shows the gating strategy for analyzing myeloid cells, including dendritic cells (DCs), myeloid-derived suppressor cells (MDSCs) and macrophages from CD45-selected cells from tumors as shown in FIGS. 14A-14H, 18A-18E, 22A-22H, 27A-27H, and 29A-29H.

FIG. 32 shows the gating strategy for analyzing T cells from CD45-selected cells from tumors as shown in FIGS. 14A-14H, 18A-18E, 22A-22H, 27A-27H, and 29A-29H.

FIG. 33 shows the gating strategy for analyzing NK cells from CD45-selected cells from tumors as shown in FIG. 15.

FIG. 34 shows the gating strategy for analyzing splenocytes from spleens shown in FIGS. 19A-19H and 29A-29H.

### DETAILED DESCRIPTION

Provided are compositions and methods for an adjuvant and CD47 blockage cancer therapy. In some aspects, the compositions and methods are used to treat a tumor that is not responsive to immunotherapy by immunologically converting the tumor into a tumor that is responsive to immunotherapy. In some aspects, the compositions provided are particles comprising a core and a ratio of a first molecule that interacts with a tumor cell and a second molecule that interacts with an antigen presenting cell. In some aspects, the first molecule that interacts with a tumor cell is a molecule that binds a tumor cell and includes, but is not limited to, an antibody, an antigen-binding fragment thereof, a nanobody, a single-chain antibody, or a polypeptide that binds a molecule present on a tumor cells. In some aspects, the second molecule interacts with an antigen presenting cell that is a dendritic cell, a macrophage or a B lymphocyte. In some aspects, the second molecule is not an antibody. In some aspects, the second molecule is a signaling lymphocytic activation molecule family member 7 (SLAMF7) polypeptide or fragment thereof. In some aspects, the fragment of SLAMF7 is a fragment that can interact with SLAMF7 on another cell. In some aspects, a particle as provided herein can bind to the surface of a tumor cell and the antigen presenting cell binding molecule SLAMF7 is thereby exposed on the surface of the tumor cell through the bound particle. Advantageously, an antigen presenting cell can bind to SLAMF7 on the tumor cell and phagocytose the tumor cell. Therefore, the particles provided herein can be used to modify tumor cells, such as, e.g., modifying a solid tumor cell that is not responsive to immunotherapy to a tumor cell that is responsive to immunotherapy (e.g., by enabling the binding interaction of an antigen presenting cell with a tumor cell through the nanoparticle comprising tumor cell binding molecules and SLAMF7 polypeptides on its surface).

### Definitions

The term “particle” as used herein refers to matter of any shape. In some aspects, the particle is a size smaller than 0.1 mm, exclusive of any molecules bound to, linked to, trapped, or encapsulated by the particle. In some aspects, the particle can have a size of about 100 μm to 0.1 nm and any size thereinbetween. A particle can comprise one or more polymers. The particle can be a nanoparticle that comprises a core region (i.e., the space between the outer dimensions of the particle) and an outer surface (i.e., the surface that defines the outer dimensions of the particle). A particle can have one or more coating layers surrounding or partially surrounding the nanoparticle core. For example, a spherical particle can have one or more concentric coating layers, each successive layer being dispersed over the outer surface of a smaller layer closer to the center of the particle. The coating layers can comprise metal ions. A particle can contain therapeutic agents (e.g., anticancer agents or immunotherapy agents) that are physically trapped within the coating layers matrix, coordinated to a metal ion of the matrix, or chemically bonded to a ligand in the coating layers. A particle can comprise a core made of a metal, including gold or iron. A particle can comprise a poly lactic acid polymer, a polyglycolic acid polymer, a poly(D,L-lactic-co-glycolic acid) co-polymer, a polyethylene glycol polymer, a poly(D,L-lactic-co-glycolic acid)-b-poly(ethylene glycol) copolymer with terminal methoxy groups (PLGA-b-mPEG), a poly(D,L-lactic-co-glycolic acid)-b-poly(ethylene glycol) copolymer with terminal N-hydroxysuccinimide ester groups (PLGA-b-PEG-NHS), a poly(D,L-lactic-co-glycolic acid)-b-poly(ethylene glycol) copolymer with terminal folici acid groups (PLGA-b-PEG-Folate), a polystyrene or combinations thereof.

The term “nanoparticle” as used herein refers to a particle having a size equal to or below 100 nm (e.g., between 1 to 100 nm in diameter). In some aspects, the nanoparticle can comprise one or more polymers. In some aspects, the nanoparticle can have a size of less than about 100 nm, less than about 80 nm, less than about 70 nm, less than about 60 nm, less than about 50 nm, less than about 40 nm, less than about 30 nm or less than about 20 nm. In some aspects, the dimension of a nanoparticle is between about 20 nm and about 100 nm (e.g., about 20, 30, 40, 50, 60, 70, 80, 90, or 100 nm). A nanoparticle can be approximately spherical. A nanoparticle can also be disc-shaped, plate-shaped, oblong, polyhedral, rod-shaped, cubic, or irregularly-shaped. A nanoparticle can comprise a core region (i.e., the space between the outer dimensions of the particle) and an outer surface (i.e., the surface that defines the outer dimensions of the particle). A nanoparticle can have one or more coating layers surrounding or partially surrounding the nanoparticle core. For example, a spherical nanoparticle can have one or more concentric coating layers, each successive layer being dispersed over the outer surface of a smaller layer closer to the center of the particle.

The terms “polymer” and “polymeric” as used herein refer to chemical structures that have repeating units (i.e., multiple copies of a given chemical substructure). Polymers can be formed from polymerizable monomers. A polymerizable monomer is a molecule that comprises one or more moieties that can react to form bonds (e.g., covalent or coordination bonds) with moieties on other molecules of polymerizable monomer. In some aspects, each polymerizable monomer molecule can bond to two or more other molecules/moieties. In some aspects, a polymerizable monomer can bond to only one other molecule, forming a terminus of the polymeric material. Polymers can be organic, or inorganic, or a combination thereof. The term “inorganic” as used herein refers to a compound or composition that contains at least some atoms other than carbon, hydrogen, nitrogen, oxygen, sulfur, phosphorous, or one of the halides. Thus, for example, an inorganic compound or composition can contain one or more silicon atoms and/or one or more metal atoms. Further, the polymeric nanoparticles can comprise a hydrophobic polymer, a hydrophilic polymer, or an amphiphilic polymer. In some aspects, the polymeric nanoparticles comprise substantially all hydrophobic polymers. In some aspects, the polymeric nanoparticles comprise substantially all hydrophilic polymers. In some aspects, the polymeric nanoparticles comprise a combination, e.g., a hydrophilic polymer and an amphiphilic polymer. Non-limiting examples of the types of the amphiphilic polymer include amphiphilic copolymer (e.g., a copolymer of a hydrophilic block coupled with a hydrophobic block), amphiphilic graft copolymer, amphiphilic block copolymer and amphiphilic random copolymer, etc. Non-limiting examples of the amphiphilic polymer include poly(lactic acid)-poly(ethylene glycol) (PLA-PEG), poly(lactic-co-glycolic acid)-poly(ethylene glycol) (PLGAPEG), poly(lactic-co-glycolic acid)-d-a-tocopheryl polyethylene glycol succinate, poly(lactic-co-glycolic acid)-ethylene oxide fumarate, poly(glycolic acid)-poly(ethylene glycol), polycaprolactone-poly(ethylene glycol), any salts thereof, any derivatives thereof and any combinations of polymers, salts and derivatives thereof.

In some aspects, the amphiphilic polymer comprises PLA-PEG, PLGA-PEG or any derivatives or salts thereof. In some aspects, the amphiphilic polymer includes PLA and PEG. A PLA polymer is derived from the condensation of lactic acid or by the ring opening polymerization of lactide. In some aspects, the weight averaged molecular weight of PLA is between 5,000 and 35,000 daltons, or any number range in between, e.g., 5,000-30,000, 8,000-30,000, 8,000-25,000, 11,000-25,000, 11,000-21,000, 14,000-21,000, 14,000-19,000, 15,000-17,000, or 17,000-19,000 daltons. In some aspects, the weight averaged molecular weight of PEG is between 1,000 and 10,000 daltons, or any number range in between, e.g., 1,000-9,000 daltons, 2,000-9,000 daltons, 2,000-8,000 daltons, 3,000-8,000 daltons, 3,000-7,000 daltons, 4,000-7,000 daltons, or 4,000-6,000 daltons. In some aspects, the weight averaged molecular weight of PEG is about 5,000 daltons, for example, between 4,500 and 5,500 daltons or between 4,000 and 6,000 daltons.

In some aspects, the PLA-PEG has a weight averaged molecular weight of between 2,000 and 60,000 daltons, or any number range in between, e.g., 3,000-60,000, 3,000-50, 45000, 5,000-50,000, 5,000-40,000, 8,000-40,000, 8,000-30,000, or 10,000-20,000 daltons. In some aspects, the PLA-PEG block co-polymer comprises a polymer chain having an about 16 kDa (e.g., 15 to 17 kDa) PLA segment attached to an about 5 kDa (e.g., 4 k to 6 kDa) PEG segment. In some aspects, the PLA-PEG block co-polymer comprises polymer chain having an about 20 kDa (e.g., 19 to 21 kDa) PLA segment attached to an about 5 kDa (e.g., 4 to 6 kDa) PEG segment. In some aspects, the PLA has a weight averaged molecular weight of about 16,000 daltons (e.g., 15 to 17 kDa) or about 20,000 daltons (e.g., between 19 to 21 kDa). In some aspects, the ratio of PLA and PEG (PLA:PEG) is between 50:5 and 10:5, or any number range in between, e.g., about 40:5, about 35:5, about 30:5, about 20:5 or about 16:5.

The term “polydispersity” as used herein refers to the average uniformity of a particle solution, where a larger polydispersity value corresponds to a larger size distribution in the particle sample.

The term “stability” as used herein includes physical and chemical stability for at least or about, e.g., 2 months, 4 months, 6 months, 8 months, 10 months, 1 year, 14 months, 16 months, 18 months, 20 months, 22 months, or 2 years at room temperature or 5° C. “Chemical stability” refers to an acceptable percentage of degradation products produced by chemical pathways such as oxidation and/or hydrolysis and/or fragmentation and/or other chemical degradation pathways. For example, a particle is considered chemically stable if no more than about 20% breakdown products are formed after one year of storage at the intended storage temperature of the composition containing the particle (e.g., room temperature). “Physical stability” refers to an acceptable percentage of aggregates (e.g., dimers, trimers and larger forms) of particles being formed. For example, a particle is considered physically stable if no more that about 15% aggregates are formed after one year of storage at the intended storage temperature of the composition containing the particle.

The term “molecule that binds a tumor cell” or any variation thereof includes any molecule that binds a molecule expressed on the surface of a tumor cell. A molecule expressed on a tumor cell includes HER2, EGFR, EGFRviii, PSMA, CEA, CA125, TROP2, CD105, GD2, GD3, VEGFR1, VEGFR2, NCAM, CD133, ADAM17, MCSP, PSCA, FOLR1, EPCAM, HER2, HER3, EGFR, PSMA, Integrin α4δ1, FAP, MUC-1, folate receptor, P glycoprotein, AFP, ALK, CD30, ErbB3, ErbB4, Mesothelin, CAIX, CD66e, cMet, EphA2, HGF/SF, TPBG, BRCA1, BORIS, FOLR1, GAGE-1, GAGE-2, GM3, gp100, MAGE-A3, MAGE1, MAGE2, MAGE3, MAGE4, MAGE6, MAGE12, MART-1, Muc1, Muc3A, Muc3B, Muc4, Muc12, Muc13, Muc15, Muc16, Muc17, Muc21, Muc22, PRLR, or CD340.

The term “molecule that binds an antigen presenting cell” or any variation thereof includes any molecule that can establish a binding interaction with an antigen presenting cells. In some aspects, the molecule that binds an antigen presenting cell comprises a full length SLAMF7 polypeptide or a fragment thereof such as, for example, an extracellular domain of SLAMF7; an Ig-like domain of SLAMF7, an Ig-like C2-type domain of SLAMF7, or any other portion of a full-length SLAMF7 polypeptide.

The term “molecular ratio” as used herein refers to the ratio of the amounts in mole of two substances. For example, the molecular ratio is the ratio of the amounts in mole of a HER2 antibody and a SLAMF7 polypeptide bound to the surface of a particle as disclosed herein.

The term “immunoglobulin” as used herein can be used interchangeably with the term “antibody”. Immunoglobulin includes full-length antibodies and any antigen binding fragment or single chains thereof. Immunoglobulins can be homo-dimeric or hetero-dimeric. Immunoglobulins and specifically naturally occurring antibodies are glycoproteins which exist as one or more copies of a Y-shaped unit, composed of four polypeptide chains. Each “Y “shape contains two identical copies of a heavy (H) chain and two identical copies of a light (L) chain, named as such by their relative molecular weights. Each light chain pairs with a heavy chain and each heavy chain pairs with another heavy chain. Covalent interchain disulfide bonds and non-covalent interactions link the chains together. Immunoglobulins and specifically naturally occurring antibodies contain variable regions, which are the two copies of the antigen binding site. Papain, a proteolytic enzyme splits the “Y” shape into three separate molecules, two so called “Fab” or “FAB” fragments (Fab=fragment antigen binding) and one so called “Fc” fragment or “Fc region” (Fc=fragment crystallizable). A Fab fragment consists of the entire light chain and part of the heavy chain. The heavy chain contains one variable region (VH) and either three or four constant regions (CH1, CH2, CH3 and CH4, depending on the antibody class or isotype). The region between the CH1 and CH2 regions is called the hinge region and permits flexibility between the two Fab arms of the Y-shaped antibody molecule, allowing them to open and close to accommodate binding to two antigenic determinants separated by a fixed distance. The “hinge region” as referred to herein is a sequence region of 6-62 amino acids in length, only present in IgA, IgD and IgG, which encompasses the cysteine residues that bridge the two heavy chains. The heavy chains of IgA, IgD and IgG each have four regions, i.e., one variable region (VH) and three constant regions (CH1-3). IgE and IgM have one variable and four constant regions (CH1-4) on the heavy chain. The constant regions of the immunoglobulins may mediate the binding to host tissues or factors, including various cells of the immune system (e.g., effector cells) and the first component (C1q) of the complement system classical pathway. Each light chain is usually linked to a heavy chain by one covalent disulfide bond. Each light chain contains one variable region (VL) and one light chain constant region. The light chain constant region is a kappa light chain constant region (IGKC) or is a lambda light chain constant region (IGLC).

The term “full length antibody” as used herein include the structure that constitutes the natural biological form of an antibody including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin regions VL and a light chain constant region, and each heavy chain comprising immunoglobulin regions VH, CH1 (Cy1), CH2 (Cy2), CH3 (Cy3) and CH4 (Cy4), depending on the antibody class or isotype). In some mammals, for example in camels and llamas, IgG antibodies may consist of only two heavy chains, each heavy chain comprising a variable region attached to the Fc region.

The terms “antibody fragment” or “immunoglobulin fragment” as used herein include, but are not limited to, (i) a region including for example a CH1, a CH2 or a CH3 region, (ii) a Fab fragment consisting of VL, VH, CL or CK and CH1 regions, including Fab″ and Fab′-SH, (iii) a Fd fragment consisting of the VH and CH1 regions, (iv) a dAb fragment (Ward E S et al., (1989) Nature, 341 (6242):544-6) which consists of a single variable region, (v) F(ab′)2 fragments, (vi) a bivalent fragment comprising two linked Fab fragments (V) single chain Fv fragments (scFv), wherein a VH region and a VL region are linked by a peptide linker which allows the two regions to associate to form an antigen binding site (Bird R E et al., (1988) Science, 242 (4877): 423-6; Huston J S et al., (1988) Proc Natl Acad Sci USA, 85 (16): 5879-83), (vii) “diabodies” or “triabodies”, multivalent or multispecific fragments constructed by gene fusion (Holliger P et al., (1993) Proc Natl Acad Sci USA, 90 (14): 6444-8; Tomlinson I & Holliger P, (2000) Methods Enzymol, 326: 461-79), (viii) scFv, diabody or region antibody fused to an Fc region and (ix) scFv fused to the same or a different antibody.

The term “polypeptide fragment” as used herein refers to a polypeptide that has an amino terminal and/or carboxy-terminal deletion, but where the remaining amino acid sequence is identical to the corresponding positions in the naturally-occurring sequence deduced, e.g., from a full length cDNA sequence. Fragments typically are at least 5, 6, 8 or 10 amino acids long, for example, at least 14 amino acids long, at least 20 amino acids long, at least 50 amino acids long, or at least 70 amino acids long.

The term “functional fragment” as used herein refers to a fragment retaining the function of a full-length antibody, such as an antigen-binding fragment, and particularly refers to an antibody fragment such as a Fv, scFv, Fab, F(ab′)2, Fab′, scFv-Fc fragment or diabody. In some aspects, a functional fragment comprises a fragment, which is capable of increasing the half-life of an antigen-binding protein by a chemical modification or by incorporation into liposomes, the chemical modification including for example, addition of a poly(alkylene) glycol such as polyethylene glycol (i.e., “polyethylene glycolylated, PEGylated”) (a PEGylated fragment being referred to as Fv-PEG, scFv-PEG, Fab-PEG, F(ab′)2-PEG or Fab′-PEG, in which “PEG” is polyethylene glycol).

The term “CD47 inhibitor” as used herein refers to any compound or composition that directly or indirectly inhibits CD47 expression and/or activity.

The term “SIRPalpha inhibitor” as used herein refers to any compound or composition that directly or indirectly inhibits SIRPalpha expression and/or activity.

The term “PD-1 inhibitor” as used herein refers to any compound or composition that directly or indirectly inhibits PD-1 expression and/or activity

The term “binding” or “specifically binding” refers to the binding of an antibody or antigen-binding protein to an antigen epitope in an in vitro assay, e.g., in a cell-based ELISA using, e.g., CHO cells which express wild-type antigens. Binding affinities can be 10⁻⁸ M or lower, and, in some aspects, are 10⁻¹³ to 10⁻⁹ M. The binding of an antibody to an antigen or FcγRIII can be studied by BIAcore assay (Pharmacia Biosensor AB, Uppsala, Sweden). The binding affinity is defined by the terms Ka (binding rate constant of an antibody in an antibody/antigen complex), Kd (dissociation constant) and equilibrium constant KD (Kd/Ka).

In some aspects, the bispecific binding protein of the present disclosure is said to specifically bind to an antigen, when the equilibrium binding constant (KD) is ≤1 mM, ≤1 μM, ≤100 nM, ≤10 nM, ≤100 pM or about 1 pM, as measured by assays such as radio-ligand binding assays, surface plasmon resonance (SPR), flow cytometry binding assay, or similar assays known to those skilled in the art.

The term “immune effector cell” or “effector cell” as used herein refers to a cell within the natural repertoire of cells in the mammalian immune system, which can be activated to affect the viability of a target cell. Immune effector cells include cells of the lymphoid lineage such as natural killer (NK) cells, T cells including cytotoxic T cells, or B cells, and cells of the myeloid lineage, such as monocytes or macrophages, dendritic cells and neutrophilic granulocytes. In some aspect, an effector cell is an NK cell, a T cell, a B cell, a monocyte, a macrophage, a dendritic cell or a neutrophilic granulocyte. The recruitment of effector cells to aberrant cells means that immune effector cells are brought in proximity to the aberrant target cells such that the effector cells can directly kill, or indirectly initiate the killing of the aberrant cells.

The terms “subject” and “patient” are used interchangeably and refer to a mammal such as a human, mouse, rat, hamster, guinea pig, rabbit, cat, dog, monkey, cow, horse, pig and the like (e.g., a patient, such as a human patient, having cancer).

The terms “treat,” “treating,” and “treatment,” as used herein refer to any type of intervention or process performed on, or administering an active agent or combination of active agents to the subject with the objective of reversing, alleviating, ameliorating, inhibiting, or slowing down or preventing the progression, development, severity or recurrence of a symptom, complication, condition or biochemical indicia associated with a disease. As used herein, “effective treatment” or “positive therapeutic response” refers to a treatment producing a beneficial effect, e.g., amelioration of at least one symptom of a disease or disorder, e.g., cancer. A beneficial effect can take the form of an improvement over baseline, including an improvement over a measurement or observation made prior to initiation of therapy according to the method. For example, a beneficial effect can take the form of slowing, stabilizing, stopping or reversing the progression of a cancer in a subject at any clinical stage, as evidenced by a decrease or elimination of a clinical or diagnostic symptom of the disease, or of a marker of cancer. Effective treatment may, for example, reduce the number of cancer cells, decrease tumor size or number of tumor cells, decrease the presence of circulating tumor cells, reduce or prevent metastases of a tumor, slow or arrest tumor cell growth and/or prevent or delay tumor recurrence or relapse.

The term “administering,” as used herein, refers to any mode of transferring, delivering, introducing, or transporting a therapeutic agent to a subject in need of treatment with such an agent. Such modes of administration include, but are not limited to, oral, topical, intravenous, intraperitoneal, intramuscular, intradermal, intranasal, and subcutaneous, intraarterial, intrathecal, intracapsular, intraorbital, intracardiac, transtracheal, subcuticular, intraarticular, subcapsular, subarachnoid, intraspinal, epidural, and intrasternal injections and infusions.

The term “effective amount” or “therapeutically effective amount” refer to an amount of an agent or combination of agents that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In some aspects, an effective amount is an amount sufficient to delay tumor development. In some aspects, an effective amount is an amount sufficient to prevent or delay tumor recurrence. In some aspects, an effective amount is administered in one or more administrations. The effective amount of a BiTN provided herein or a composition comprising the same may: (i) reduce the number of cancer cells; (ii) reduce the infiltration sites of cancer cells; (iii) inhibit, retard, slow to some extent and may stop cancer cell infiltration into peripheral organs; (iv) inhibit cancer metastasis; (v) inhibit cancer growth; (vi) prevent or delay occurrence and/or recurrence of a cancer; and/or (vii) relieve to some extent one or more of the symptoms associated with the cancer.

The term “cancer” as used herein refers to any cancer that can be made responsive to an immunotherapy. Examples of cancers include, but are not limited to, breast cancer, brain cancer, prostate cancer, lung cancer, colorectal cancer, skin cancer, head and neck cancer, or pancreatic cancer.

The term “additional cancer therapy” as used herein refers to a radiation therapy, surgery, chemotherapy, gene therapy, DNA therapy, viral therapy, RNA therapy, immunotherapy, bone marrow transplantation, nanotherapy, additional monoclonal antibody therapy, or a combination of the foregoing. The additional therapy may be in the form of adjuvant or neoadjuvant therapy.

In some aspects, the additional cancer therapy is a small molecule enzymatic inhibitor or anti-metastatic agent. In some aspects, the additional cancer therapy is radiation therapy. In some aspects, the additional cancer therapy is a therapy targeting the PBK/AKT/mTOR pathway, a HSP90 inhibitor, a tubulin inhibitor, an apoptosis inhibitor, and/or a chemopreventative agent. The additional cancer therapy may be one or more of the chemotherapeutic agents known in the art. For example, a chemotherapeutic agents may be a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Examples of chemotherapeutic agents include, but are not limited to, alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins including bullatacin and bullatacinone; a camptothecin including the synthetic analogue topotecan; bryostatin; callystatin; CC-1065 including its adozelesin, carzelesin and bizelesin synthetic analogues; cryptophycins including cryptophycin 1 and cryptophycin 8; dolastatin; duocarmycin including the synthetic analogues, KW-2189 and CB 1-TMl; eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and raninmustine; antibiotics, such as the enediyne antibiotics, e.g., calicheamicin, including calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin including morpholinodoxorubicin, cyanomorpholino-doxorubicin, 2-pyrrolinodoxorubicin and deoxydoxorubicin; epirubicin; esorubicin; idarubicin; marcellomycin; mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; itoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes including T-2 toxin, verracurin A, roridin A and anguidine; urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan, e.g., CPT-11; topoisomerase inhibitor RFS 2000; difluorometlhylornithine (DMFO); retinoids, such as retinoic acid; capecitabine; carboplatin, procarbazine, plicomycin, gemcitabien, navelbine, farnesylprotein transferase inhibitors, transplatinum, and pharmaceutically acceptable salts, acids, or derivatives of any of the above.

The dosing regimen of a composition comprising a BiTN and an additional cancer therapy can be determined based on the overall health of the subject to be treated and standard dosing guidelines for cancer therapy and/or combination therapies comprising antibody therapy and additional antibody or non-antibody cancer therapy.

### Multi or Bispecific Tumor Transforming Nanoparticles (BiTNs)

Provided are materials and methods for treating tumors by immunologically converting a tumor that is not responsive to immunotherapy into a tumor that is responsive to immunotherapy. In some aspects, provided are particles comprising a core and a ratio of a first molecule that interacts with a tumor cell and a second molecule that interacts with antigen presenting cells (APCs). In some aspects, the first molecule that interacts with a tumor cells is a molecule that binds a tumor cell and includes, but is not limited to, an antibody, an antigen-binding fragment thereof, a nanobody, a single-chain antibody, or a polypeptide that binds a molecule present on a tumor cells.

In some aspects, the first molecule can be an anti-cancer antigen antibody (e.g., an anti-human epidermal growth factor receptor 2 (HER2) antibody such as a Herceptin® antibody (trastuzumab) comprising a heavy chain of SEQ ID NO: 2 and a light chain of SEQ ID NO: 16). In some aspects, the second molecule can be a molecule that has the ability to bind to an APC such as a SLAMF7 amino acid sequence. Advantageously, a particle as provided herein can transform a tumor that was previously unresponsive to an immunotherapy, e.g., a CD47 blockage therapy, into a tumor that is responsive to the immunotherapy. For example, a bispecific particle comprising a first molecule that is a HER2 antibody and a second molecule that is SLAMF7 when contacted to a tumor cell that is unresponsive to immunotherapy can transform such tumor cell into a tumor cell that is responsive to an immunotherapy, e.g., a CD47 blockage therapy. The bispecific particles is therefore a bispecific tumor transforming nanoparticle (BiTN).

Such BiTNs can have the ability to facilitate the phagocytosis of cancer cells by APCs within a mammal's body and to activate downstream adaptive immune responses (e.g., T cell responses) to treat cancer. In addition, the modular design of the BiTNs provided herein (e.g., a two component modular design or three component modular design) can allow clinicians to readily target tumors of different types or tumors of the same type but from different patients to personalize their therapy. In some aspects, the nanoparticles provided herein can comprise more than one cancer cell binding molecule and one APC binding molecule. In some aspects, the nanoparticles provided herein can comprise one cancer cell binding molecule and more than one APC binding molecule. In some aspects, the nanoparticles provided herein can comprise more than one cancer cell binding molecule and more than one APC binding molecule.

In some aspects, the cancer cell binding molecule can bind to HER2, EGFR, EGFRviii, PSMA, CEA, CA125, TROP2, CD105, GD2, GD3, VEGFR1, VEGFR2, NCAM, CD133, ADAM17, MCSP, PSCA, FOLR1, EPCAM, HER3, EGFR, PSMA, Integrin α4β1, FAP, MUC-1, P glycoprotein, AFP, ALK, CD30, ErbB3, ErbB4, Mesothelin, CAIX, CD66e, cMet, EphA2, HGF/SF, TPBG, BRCA1, BORIS, FOLR1, GAGE-1, GAGE-2, GM3, gp100, MAGE-A3, MAGE1, MAGE2, MAGE3, MAGE4, MAGE6, MAGE12, MART-1, Muc1, Muc3A, Muc3B, Muc4, Muc12, Muc13, Muc15, Muc16, Muc17, Muc21, Muc22, PRLR, or CD340. In some aspects, the cancer cell binding molecule is not an antibody. In some aspects, the cancer cell binding molecule is a polypeptide that binds to HER2, EGFR, EGFRviii, PSMA, CEA, CA125, TROP2, CD105, GD2, GD3, VEGFR1, VEGFR2, NCAM, CD133, ADAM17, MCSP, PSCA, FOLR1, EPCAM, HER2, HER3, EGFR, PSMA, Integrin α4β1, FAP, MUC-1, P glycoprotein, AFP, ALK, CD30, ErbB3, ErbB4, Mesothelin, CAIX, CD66e, cMet, EphA2, HGF/SF, TPBG, BRCA1, BORIS, FOLR1, GAGE-1, GAGE-2, GM3, gp100, MAGE-A3, MAGE1, MAGE2, MAGE3, MAGE4, MAGE6, MAGE12, MART-1, Muc1, Muc3A, Muc3B, Muc4, Muc12, Muc13, Muc15, Muc16, Muc17, Muc21, Muc22, PRLR, or CD340. In some aspects, the cancer cell binding molecule is a RGD peptide. In some aspects, the cancer binding molecule is a vitamin including, but not limited to folic acid, or a glucose derivative including, but not limited to, mannose or dehydroabietic acid.

In some aspects, the second molecule that interacts with an antigen binding cell interacts with a macrophage, dendritic cell, B lymphocyte, and/or a natural killer cell. In some aspects, the second molecule that interacts with an antigen presenting cell is a signaling lymphocyte activation molecule family member 7 (SLAMF7) polypeptide or a fragment thereof. In some aspects, the second molecule that interacts with an antigen presenting cell is not an antibody.

In some aspects, the nanoparticle comprises a core and a first and a second molecule covalently bound to the core. In some aspects, the core of the particle comprises a polymer including, but not limited to, a poly lactic acid polymer, a polyglycolic acid polymer, a poly(D,L-lactic-co-glycolic acid) co-polymer, a polyethylene glycol polymer, a poly(D,L-lactic-co-glycolic acid)-b-poly(ethylene glycol) with terminal methoxy groups (PLGA-b-mPEG), a poly(D,L-lactic-co-glycolic acid)-b-poly(ethylene glycol) with terminal N-hydroxysuccinimide ester groups (PLGA-b-PEG-NHS) or a poly(D,L-lactic-co-glycolic acid)-b-poly(ethylene glycol) with terminal folic acid groups (PLGA-b-PEG-Folate). In some aspects, the core of the particle comprises a poly(D,L-lactic-co-glycolic acid)-b-poly(ethylene glycol) with terminal methoxy groups (PLGA-b-mPEG), a poly(D,L-lactic-co-glycolic acid)-b-poly(ethylene glycol) with terminal N-hydroxysuccinimide ester groups (PLGA-b-PEG-NHS) or a poly(D,L-lactic-co-glycolic acid)-b-poly(ethylene glycol) with terminal folic acid groups (PLGA-b-PEG-Folate). In some aspects, the core of the particle comprises a mixture comprising a PLGA-b-mPEG, a PLGA-b-PEG-NHS and/or a PLGA-b-PEG-Folate at different ratios.

In some aspects, the ratio of PLA and PEG (PLA:PEG) in the nanoparticle is about between 50:5 and about 10:5, or any number range in between, e.g., about 40:5, about 35:5, about 30:5, about 20:5 or about 16:5.

In some aspects, nanoparticles comprising amine-reactive polymer cores are prepared and combined with a first and a second molecule as described herein. In some aspects, the first and second molecule are added at a ratio to the amine-reactive polymer core to generate a nanoparticle with a predetermined quantity of a first molecule and a predetermined quantity of the second molecule conjugated to the surface of the polymer core. In some aspects, the ratio of the first and the second molecule is a ratio of about 1:100; about 1:95; about 1:90; about 1:85; about 1:80; about 1:75; 1:70; about 1:65; about 1:60; about 1:55; about 1:50; about 1:45; about 1:40; about 1:35; about 1:30; about 1:25; about 1:20; about 1:19; about 1:18; about 1:17; about 1:16; about 1:15; about 1:14; about 1:13; about 1:12; about 1:11; about 1:10; about 1:9.5; about 1:9; about 1:8.5; about 1:8; about 1:7.5; about 1:7; about 1:6.5; about 1:6; about 1:5.5; about 1:5; about 1:4.5; about 1:4; about 1:3.5; about 1:3; about 1:2.9; about 1:2.8; about 1:2.7; about 1:2.6; about 1:2.5; about 1:2.4; about 1:2.3; about 1:2.2; about 1:2.1; about 1:2; about 1:1.9; 1:1.8; about 1:1.7; about 1:1.6; about 1:1.5; about 1:1.4; about 1:1.3; about 1:1.2; about 1:1.1; about 1:1; about 1:0.9; about 1:0.8; about 1:0.7; about 1:0.6; about 1:0.5; about 1:0.4; about 1:0.3; about 1:0.2; about 1:0.1; about 0.1:1; about 0.2:1; about 0.3:1; about 0.4:1; about 0.5:1; about 0.6:1; about 0.7:1; about 0.8:1; about 0.9:1; about 1.1:1; about 1.2:1; about 1.3:1; about 1.4:1; about 1.5:1; about 1.6:1; about 1.7:1; about 1.8:1; about 1.9:1; about 2:1; about 2.1:1; about 2.2:1; about 2.3:1; about 2.4:1; about 2.5:1; about 2.6:1; about 2.7:1; about 2.8:1; about 2.9:1; about 3:1; about 3.5:1; about 4:1; about 4.5:1; about 5:1; about 5.5:1; about 6:1; about 6.5:1; about 7:1; about 7.5:1; about 8:1; about 8.5:1; about 9:1; about 9.5:1; about 10:1; about 11:1; about 12:1; about 13:1; about 14:1; about 15:1; about 16:1; about 17:1; about 18:1; about 19:1; about 20:1; about 25:1; about 30:1; about 35:1; about 40:1; about 45:1; about 50:1; about 55:1; about 60:1; about 65:1; about 70:1; about 75:1; about 80:1; about 85:1; about 90:1; about 95:1; or about 100:1.

In some aspects, the ratio of the first and the second molecule is a ratio of about 1:10 to about 10:1. In some aspects, the ratio of the first and the second molecule is a ratio of about 1:3 to about 3:1.

In some aspects, the particle comprises about 0 to about 2100 first molecules. In some aspects, the particle comprises about 2 to about 1000 first, tumor cell binding molecules or about 3 to about 500, or about 5 to about 480, about 10 to about 460, about 20 to about 440, about 50 to about 420, about 100 to about 400, about 110 to about 380, about 120 to about 360, about 140 to about 340, about 160 to about 320, or about 10-20, 21-30, 31-40, 41-50, 51-60, 61-70, 71-80, 81-90, 91-100, 101-110, 111-120, 121-130, 131-140, 141-150, 151-160, 161-170, 171-180, 181-190, 191-200, 201-210, 211-220, 221-230, 231-240, 241-250, 251-260, 261-270, 271-280, 281-290, 291-300, 301-310, 311-320, 321-330, 331-340, 341-350, 351-360, 361-370, 371-380, 381-390, 391-400, 401-410, 411-420, 421-430, 431-440, 441-450, 451-460, 461-470, 471-480, 481-490, 491-500, 501-510, 511-520, 521-530, 531-540, 541-550, 551-560, 561-570, 571-580, 581-590, or 591-600. In some aspects, the particle comprises 100-500 first, tumor cell binding molecules. In some aspects, the particle comprises 120-400 first, tumor cell binding molecules. In some aspects, the particle comprises 144-360 first, tumor cell binding molecules. In some aspects, the particle comprises 144 first, tumor cell binding molecules. In some aspects, the particle comprises 250 first, tumor cell binding molecules. In some aspects, the particle comprises 360 first, tumor cell binding molecules. In some aspects, the particle comprises 450 first, tumor cell binding molecules. In some aspects, the first molecule is an antibody fragment. In some aspects, the antibody fragment is an antibody fragment lacking an Fc region. In some aspects, the antibody fragment lacking an Fc region is a F(ab′)2 fragment. In some aspects, the antibody fragment lacking an Fc region is a Fab fragment. In some aspects, the first molecule is a single-chain antibody. In some aspects, the first molecule is a nanobody. In some aspects, the first molecule is a tumor cell binding polypeptide that is not an antibody or fragment thereof.

In some aspects, the first molecule comprises more than one different cancer cell binding molecule. In some aspects, between 2 and 20 different cancer cell binding molecules. In some aspects, the between 2 and 10 different cancer binding molecules together make up the amount of the first, cancer binding molecule for the purpose of determining the ratio of first cancer binding molecule to second APC binding molecule as described above.

In some aspects, the second molecule is a full-length SLAMF7 polypeptide. In some aspects, the second molecule is an engineered SLAMF7 polypeptide. In some aspects, the engineered SLAMF7 polypeptide is mature SLAMF7 polypeptide. In some aspects, the engineered SLAMF7 polypeptide is a N-terminally truncated SLAMF7 polypeptide. In some aspects, the engineered SLAMF7 polypeptide is an extracellular domain of a SLAMF7 polypeptide. In some aspects, the engineered SLAMF7 polypeptide is an immunoglobulin-like domain of a SLAMF7 polypeptide. In some aspects, the engineered SLAMF7 polypeptide is an immunoglobulin-like V-type domain of a SLAMF7 polypeptide. In some aspects, the engineered SLAMF7 polypeptide is an immunoglobulin-like C2-type domain of a SLAMF7 polypeptide.

In some aspects, the particle comprises about 0 to about 2100 second molecules.

In some aspects, the particle comprises about 2 to about 2000 second, antigen presenting cell binding molecules or about 3 to about 1950, or about 5 to about 1900, about 10 to about 1850, about 20 to about 1800, about 50 to about 1750, about 100 to about 1700, about 110 to about 1650, about 120 to about 1600, about 140 to about 1550, about 160 to about 1500 or about 10-20, 21-30, 31-40, 41-50, 51-60, 61-70, 71-80, 81-90, 91-100, 101-110, 111-120, 121-130, 131-140, 141-150, 151-160, 161-170, 171-180, 181-190, 191-200, 201-210, 211-220, 221-230, 231-240, 241-250, 251-260, 261-270, 271-280, 281-290, 291-300, 301-310, 311-320, 321-330, 331-340, 341-350, 351-360, 361-370, 371-380, 381-390, 391-400, 401-410, 411-420, 421-430, 431-440, 441-450, 451-460, 461-470, 471-480, 481-490, 491-500, 501-510, 511-520, 521-530, 531-540, 541-550, 551-560, 561-570, 571-580, 581-590, 591-600, 601-610, 611-620, 621-630, 631-640, 641-650, 651-660, 661-670, 671-680, 681-690, 691-700, 701-710, 711-720, 721-730, 731-740, 741-750, 751-760, 761-770, 771-780, 781-790, 791-800, 801-810, 811-820, 821-830, 831-840, 841-850, 851-860, 861-870, 871-880, 881-890, 891-900, 901-910, 911-920, 921-930, 931-940, 941-950, 951-960, 961-970, 971-980, 981-990, 991-1000, 1001-1010, 1011-1020, 1021-1030, 1031-1040, 1041-1050, 1051-1060, 1061-1070, 1071-1080, 1081-1090, 1091-1100, 1101-1110, 1111-1120, 1121-1130, 1131-1140, 1141-1150, 1151-1160, 1161-1170, 1171-1180, 1181-1190, 1191-1200, 1201-1210, 1211-1220, 1221-1230, 1231-1240, 1241-1250, 1251-1260, 1261-1270, 1271-1280, 1281-1290, 1291-1300, 1301-1310, 1311-1320, 1321-1330, 1331-1340, 1341-1350, 1351-1360, 1361-1370, 1371-1380, 1381-1390, 1391-1400, 1401-1410, 1411-1420, 1421-1430, 1431-1440, 1441-1450, 1451-1460, 1461-1470, 1471-1480, 1481-1490, 1491-1500, 1501-1510, 1511-1520, 1521-1530, 1531-1540, 1541-1550, 1551-1560, 1561-1570, 1571-1580, 1581-1290, 1591-1600, 1601-1610, 1611-1620, 1621-1630, 1631-1640, 1641-1650, 1651-1660, 1661-1670, 1671-1680, 1681-1690, 1691-1700. In some aspects, the particle comprises 500-1800 second, antigen presenting cell binding molecules. In some aspects, the particle comprises 500-1600 second, antigen presenting cell binding molecules. In some aspects, the particle comprises 522-1564 second, antigen presenting cell binding molecules. In some aspects, the particle comprises 522 second, antigen presenting cell binding molecules. In some aspects, the particle comprises 1085 second, antigen presenting cell binding molecules. In some aspects, the particle comprises 1564 second, antigen presenting cell binding molecules. In some aspects, the particle comprises 2000 second, antigen presenting cell binding molecules. In some aspects, the SLAMF7 polypeptide and/or fragment thereof is a human SLAMF7 polypeptide or fragment thereof.

In some aspects, the SLAMF7 polypeptide and/or fragment thereof is a mouse SLAMF7 polypeptide or fragment thereof.

In some aspects, the bispecific tumor-transforming nanoparticle comprises about 140 to about 460 first molecules and about 500 to about 2100 second molecules, wherein the first molecule is a HER2 antibody and the second molecule is full-length SLAMF7 polypeptide.

In some aspects, the first molecule is a HER2 antibody comprising a heavy chain of SEQ ID NO: 2 and a light chain of SEQ ID NO: 16.

In some aspects, the bispecific tumor-transforming nanoparticle comprises about 350 HER2 antibodies and about 550 SLAMF7 polypeptides. In some aspects, the bispecific tumor-transforming nanoparticle comprises about 240 HER2 antibodies and about 1100 SLAMF7 polypeptides. In some aspects, the bispecific tumor-transforming nanoparticle comprises about 140 HER2 antibodies and about 1600 SLAMF7 polypeptides. In some aspects, the bispecific tumor-transforming nanoparticle comprises about 50 HER2 antibodies and about 2000 SLAMF7 polypeptides

In some aspects, the particle is fluorescently labeled. For example, coumarin-6 can be encapsulated in the particle during the process of conjugating the tumor cell binding molecule and the SLAMF7 polypeptide to the particle.

In some aspects, a BiTN comprises a first molecule that is a folate. In some aspects, the core of the particle comprises a poly(D,L-lactic-co-glycolic acid)-b-poly(ethylene glycol) with terminal folic acid groups (PLGA-b-PEG-Folate). In some aspects, the second molecule is added at a ratio of folate to second molecule to the PLGA-b-PEG-Folate to prepare a BiTN that comprise folate and the second molecule on the surface.

In some aspects, the BiTN comprises a folate and a SLAMF7 polypeptide covalently attached to the surface.

In some aspects, the ratio of the folate and the SLAMF7 polypeptide is a ratio of about 100:1 to about 1:100; or about 1:1, about 1.2:1, about 1.3:1, about 1.4:1, about 1.5:1, about 1.6:1, about 1.7:1, about 1.8:1 about 1.9:1, about 2:1, about 2.1:1, about 2.2:1, about 2.3:1, about 2.4:1, about 2.5:1, about 2.6:1, about 2.7:1, about 2.8:1, about 2.9, about 3:1, about 3.1:1, about 3.2:1, about 3.3:1, about 3.4:1, about 3.5:1, about 3.6:1, about 3.7:1, about 3.8:1, about 3.9:1, about 4:1, about 4.1:1, about 4.2:1, about 4.3:1, about 4.4:1, about 4.5:1, about 4.6:1, about 4.7:1, about 4.8:1, about 4.9:1, about 5:1, about 5.1:1, about 5.2:1, about 5.3:1, about 5.4:1, about 5.5:1, about 5.6:1, about 5.7:1; about 5.8:1, about 5.9:1, about 6:1; about 6.5:1; about 7:1; about 7.5:1; about 8:1; about 8.5:1; about 9:1; about 9.5:1; about 10:1; about 11:1; about 12:1; about 13:1; about 14:1; about 15:1; about 16:1; about 17:1; about 18:1; about 19:1; about 20:1; about 25:1; about 30:1; about 35:1; about 40:1; about 45:1; about 50:1; about 55:1; about 60:1; about 65:1; about 70:1; about 75:1; about 80:1; about 85:1; about 90:1; about 95:1; or about 1:20; about 1:19; about 1:18; about 1:17; about 1:16; about 1:15; about 1:14; about 1:13; about 1:12; about 1:11; about 1:10; about 1:9.5; about 1:9; about 1:8.5; about 1:8; about 1:7.5; about 1:7; about 1:6.5; about 1:6; about 1:5.5; about 1:5; about 1:4.5; about 1:4; about 1:3.5; about 1:3; about 1:2.9; about 1:2.8; about 1:2.7; about 1:2.6; about 1:2.5; about 1:2.4; about 1:2.3; about 1:2.2; about 1:2.1; about 1:2; about 1:1.9; about 1:1.8; about 1:1.7; about 1:1.6; about 1:1.5; about 1:1.4; about 1:1.3; about 1:1.2; about 1:1.1.

Advantageously, the particle described herein activates an innate pro-phagocytic response immune response when administered to a subject in need of an innate pro-phagocytic immune response. Without wanting to be bound by theory, it is believed that the presence of the particle described herein bound to a tumor cell through the first molecule of the particle attracts and activates an antigen presenting cell through interaction fo the antigen presenting cell with the second molecule, i.e., the SLAMF7 polypeptide or fragment thereof present on the surface of the tumor cell after binding of the particle through its first molecule to the surface of the tumor cell. Advantageously, the engagement of an antigen presenting cell with the tumor cell through a tumor cell binding-SLAMF7-particle described herein activates the antigen presenting cell and activates the phagocytosis of the tumor cell and presentation of tumor cell antigens on the antigen presenting cell to activate an antigen specific response in T cells and, thereby bridging the innate and antigen specific immune systems for an enhanced anti-tumor immune response.

### Compositions

Further provided are compositions comprising a particle as described herein. In some aspects, the compositions comprise the particle and a pharmaceutical excipient.

In some aspects, a composition as described herein further comprises a treatment with molecules that interfere with the interaction between PD-1 and PD-L1 (e.g., anti-PD-1 antibodies or anti-PD-L1 antibodies), molecules that interfere with the interaction between CD47 and SIRPα (e.g., anti-CD47 antibodies or anti-SIRPα antibodies or SIRPα binding peptides), molecules that block CTLA-4, molecules that stimulate innate immune responses (e.g., NK cell activators, stimulators of interferon gene agonists such as MK-1454) and/or CAR T cell immunotherapies.

In some aspects, the composition further comprises a CD47 blocking agent.

In some aspects, the molecule that modulates CD47 expression and/or activity includes, but is not limited to, molecules such as, siRNA, antisense molecule, protein, peptide, small molecule, antibody, etc. In some aspects, the CD47 blocking agent is selected from a CD47 antibody, a fragment thereof, a CD47 nanobody and a CD47 binding polypeptide.

Examples of molecules that can be used to block CD47 include, but are not limited to, TTI-621 from Trillium, Hu5F9-G4 (Stanford), CC-90002 (Celgene/InhibRx), NI-1701 (Novimmune SA), or inhibitors listed in the following documents, which are incorporated herein by reference, WO2014093678 Therapeutic CD47 antibodies; US20140161805 Methods for manipulating phagocytosis mediated by CD47; US20140161825 Methods of treating acute myeloid leukemia by blocking CD47; US20120189625 Compositions and methods for treating hematological cancers targeting the SIRPalpha-CD47 interaction; US20120156724 Humanized anti-CD47 antibody; US20130142786 Humanized and chimeric monoclonal antibodies to CD47. In some aspects, the SIRPalpha-CD47 checkpoint inhibitors include anti-CD47 antibodies or antibody fragments and non-Fe-receptor binding anti-CD47 inhibitors.

In some aspects, the CD47 blockage therapy includes a SIRPalpha Inhibitor. In some aspects, a molecule that modulates SIRPalpha expression and/or activity includes, but is not limited to, siRNA, antisense molecule, protein, peptide, small molecule, antibody, etc. In some aspects, the molecule that modulates SIRPalpha expression and/or activity is an anti-SIRPalpha antibody or antibody fragment, or a non-Fe-receptor binding anti-SIRPalpha inhibitor.

In some aspects, the composition further comprises a molecule blocking transmembrane programmed cell death 1 protein (PD1) and/or its ligand PD1 ligand (PD-L1) and includes, but is not limited to, a siRNA, antisense molecule, protein, peptide, small molecule, or a PD1 antibody and/or a PD-LI antibody. In some aspects, the antibody is pembrolizumab, Nivolumab, pidilizumab, atezolizumab, avelumab, durvalumab; or a small proteins engineered to target PD-L1 such as Aflimer™ biotherapeutic from Avacta Life Sciences.

In some aspects, the reduction in CD47 and/or SIRPalpha and/or PD1/PD-L1 expression level or activity level is of at least 10% lower, in a further embodiment, at least 15% lower, in a further embodiment, at least 20% lower, in a further embodiment of at least 30%, in a further embodiment of at least 40%, in a further embodiment of at least 50% lower, in a further embodiment of at least 60% lower, in a further embodiment of at least 70% lower, in a further embodiment of at least 80%, in a further embodiment of at least 90%, in a further embodiment of 100% (complete inhibition).

In some aspects, the combination therapy comprises a particle of the disclosure in combination with an immune checkpoint inhibitor (e.g., a CD47 blocking agent), and optionally, a further immune checkpoint inhibitor (e.g., an anti-PD-1 or anti-PD-L1 antibody). In some aspects, the further immune checkpoint inhibitor can comprise a further CD47 blocking agent. In some aspects, the further immune checkpoint inhibitor is not a CD47 blocking agent.

In some aspects, the composition comprises a tumor cell binding fragment of a tumor cell binding molecule, a SLAMF7 polypeptide and a CD47 blocking agent. In some aspects, the composition comprises a tumor cell binding fragment of a tumor cell binding molecule, a SLAMF7 polypeptide, a CD47 blocking agent and an unconjugated particle. In some aspects, the composition further comprises a pharmaceutical excipient.

In some aspects, the compositions comprise a tumor cell binding molecule, a SLAMF7 polypeptide and, optionally, an unconjugated particle. In some aspects, the compositions further, optionally, comprise a non-tumor cell binding immunoglobulin. In some aspects, the composition comprises a tumor cell binding molecule, a SLAMF7 polypeptide, a CD47 blocking agent and, optionally, an unconjugated particle. In some aspects, the composition further comprises a pharmaceutical excipient.

In some aspects, the compositions comprise a BiTN and a non-tumor binding immunoglobulin. In some aspects, the compositions comprise a BiTN, a non-tumor binding immunoglobulin and a pharmaceutical excipient.

In some aspects, the CD47 blocking agent is administered at a dose of about 0.1 mg/kg to about 60 mg/kg; or about 0.2 mg/kg to about 58 mg/kg, about 0.3 mg/kg to about 55 mg/kg, about 0.4 mg/kg to about 54 mg/kg, about 0.5 mg/kg to about 52 mg/kg, about 0.9 mg/kg to about 50 mg/kg; or about 0.1 mg/kg to about 20 mg/kg, about 21 mg/kg to about 30 mg/kg, about 31 mg/kg to about 40 mg/kg, about 41 mg/kg to about 50 mg/kg, about 51 mg/kg to about 60 mg/kg; or about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg about 19 mg/kg, about 20 mg/kg, about 22 mg/kg, about 24 mg/kg, about 26 mg/kg, about 28 mg/kg, about 30 mg/kg, about 32 mg/kg, about 34 mg/kg, about 36 mg/kg, about 38 mg/kg, about 40 mg/kg, about 42 mg/kg, about 44 mg/kg, about 46 mg/kg, about 48 mg/kg, about 50 mg/kg, about 52 mg/kg, about 53 mg/kg, about 54 mg/kg, about 55 mg/kg, about 56 mg/kg, about 57 mg/kg, about 58 mg/kg, about 59 mg/kg, or about 60 mg/kg.

In some aspects, the bispecific tumor-transforming nanoparticle is administered at a dose of about 0.1 mg/kg to about 60 mg/kg; or about 0.2 mg/kg to about 58 mg/kg, about 0.3 mg/kg to about 55 mg/kg, about 0.4 mg/kg to about 54 mg/kg, about 0.5 mg/kg to about 52 mg/kg, about 0.9 mg/kg to about 50 mg/kg; or about 0.1 mg/kg to about 20 mg/kg, about 21 mg/kg to about 30 mg/kg, about 31 mg/kg to about 40 mg/kg, about 41 mg/kg to about 50 mg/kg, about 51 mg/kg to about 60 mg/kg; or about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg about 19 mg/kg, about 20 mg/kg, about 22 mg/kg, about 24 mg/kg, about 26 mg/kg, about 28 mg/kg, about 30 mg/kg, about 32 mg/kg, about 34 mg/kg, about 36 mg/kg, about 38 mg/kg, about 40 mg/kg, about 42 mg/kg, about 44 mg/kg, about 46 mg/kg, about 48 mg/kg, about 50 mg/kg, about 52 mg/kg, about 53 mg/kg, about 54 mg/kg, about 55 mg/kg, about 56 mg/kg, about 57 mg/kg, about 58 mg/kg, about 59 mg/kg, or about 60 mg/kg.

In some aspects, the compositions provided herein further comprise a PD-1 and/or PD-L1 blocking agent. Advantageously, the anti-tumor response in a subject having a tumor is enhanced when the BiTN is administered to a subject together with a PD-1 and/or PD-L1 blocking agent. In some aspects, the PD-1 and/or PD-L1 blocking agent can be a PD-1 antibody, a fragment thereof, a PD-1 nanobody and a PD-1 binding polypeptide.

In some aspects, the PD-1 and/or PD-L1 blocking agent is administered at a dose of about 0.1 mg/kg to about 60 mg/kg; or about 0.2 mg/kg to about 58 mg/kg, about 0.3 mg/kg to about 55 mg/kg, about 0.4 mg/kg to about 54 mg/kg, about 0.5 mg/kg to about 52 mg/kg, about 0.9 mg/kg to about 50 mg/kg; or about 0.1 mg/kg to about 20 mg/kg, about 21 mg/kg to about 30 mg/kg, about 31 mg/kg to about 40 mg/kg, about 41 mg/kg to about 50 mg/kg, about 51 mg/kg to about 60 mg/kg; or about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg about 19 mg/kg, about 20 mg/kg, about 22 mg/kg, about 24 mg/kg, about 26 mg/kg, about 28 mg/kg, about 30 mg/kg, about 32 mg/kg, about 34 mg/kg, about 36 mg/kg, about 38 mg/kg, about 40 mg/kg, about 42 mg/kg, about 44 mg/kg, about 46 mg/kg, about 48 mg/kg, about 50 mg/kg, about 52 mg/kg, about 53 mg/kg, about 54 mg/kg, about 55 mg/kg, about 56 mg/kg, about 57 mg/kg, about 58 mg/kg, about 59 mg/kg, or about 60 mg/kg.

In some aspects, the composition comprises a BiTN and a PD-1 and/or PD-L1 blocking agent.

In some aspects, the composition comprises a BiTN, a CD47 blocking agent and a PD-1 and/or PD-L1 blocking agent.

In some aspects, compositions are provided that comprise a BiTN, a CD47 blocking agent, and, optionally, a PD-1/PD-L1 blocking agent.

In some aspects, compositions are provided that comprise increasing concentrations of the BiTN, a constant dose of a CD47 blocking agent, and optionally, a constant dose of a PD-1/PD-L1 blocking agent.

In some aspects, compositions are provided that comprise constant dose of a BiTN, an increasing concentration of a CD47 blocking agent, and optionally, a constant dose of a PD-1/PD-L1 blocking agent.

In some aspects, compositions are provided that comprise constant dose of a BiTN, a constant dose of a CD47 blocking agent, and optionally, an increasing concentration of a PD-1/PD-L1 blocking agent.

In some aspects, a composition comprises a BiTN, a second composition comprises a CD47 blocking agent, and a third composition comprises a PD-1/PD-L1 blocking agent.

In some aspects, a composition comprises a BiTN and a CD47 blocking agent, and a second composition comprises a PD-1/PD-L1 blocking agent.

In some aspects, a composition comprises a BiTN and a PD-1/PD-L1 blocking agent, and a second composition comprises a CD47 blocking agent.

In some aspects, a composition comprises a BiTN and a second composition comprises a PD-1/PD-L1 blocking agent and a CD47 blocking agent.

In some aspects, the composition comprising a BiTN and the composition comprising a CD47 blocking agent and/or a PD-1/PD-L1 blocking agent are administered at the same time.

In some aspects, provided is a composition comprises a particle that has only folate attached to its surface and a CD47 blocking agent. In some aspects, the composition comprises a particle that has only SLAMF7 attached to its surface and a CD47 blocking agent. In some aspects, the composition comprises folate, a SLAMF7 polypeptide, an unconjugated particle, and a CD47 blocking agent.

In some aspects, the composition comprises a bispecific tumor transforming nanoparticle that comprises folate and SLAMF7 polypeptide attached to its surface.

In some aspects, the composition comprises a bispecific tumor transforming nanoparticle that comprises folate and SLAMF7 polypeptide attached to its surface and a CD47 blocking agent. Advantageously, the composition comprises a bispecific tumor transforming nanoparticle that comprises folate and SLAMF7 polypeptide attached to its surface and a CD47 blocking agent when administered to a subject having a tumor leads to significantly increased tumor cell phagocytosis compared to a subject administered only the CD47 blocking agent.

In some aspects, compositions are provided that comprise increasing concentrations of a bispecific tumor transforming nanoparticle that comprises folate and SLAMF7 polypeptide attached to its surface and a constant dose of CD47 blocking agent.

In some aspects, compositions are provided that comprise constant doses of a bispecific tumor transforming nanoparticle that comprises folate and SLAMF7 polypeptide attached to its surface and increasing concentrations of the CD47 blocking agent.

In some aspects, compositions are provided that comprise a bispecific tumor transforming nanoparticle that comprises folate and SLAMF7 polypeptide attached to its surface, a CD47 blocking agent, and, optionally, a PD1/PD-L1 blocking agent.

In some aspects, compositions are provided that comprise a bispecific tumor transforming nanoparticle that comprises folate and SLAMF7 polypeptide attached to its surface, a CD47 blocking agent, and a PD1/PD-L1 blocking agent.

In some aspects, compositions are provided that comprise increasing concentrations of a bispecific tumor transforming nanoparticle that comprises folate and SLAMF7 polypeptide attached to its surface, a constant dose of a CD47 blocking agent, and optionally, a constant dose of a PD1/PD-L1 blocking agent.

In some aspects, compositions are provided that comprise constant dose of a bispecific tumor transforming nanoparticle that comprises folate and SLAMF7 polypeptide attached to its surface, an increasing concentration of a CD47 blocking agent, and optionally, a constant dose of a PD1/PD-L1 blocking agent.

In some aspects, compositions are provided that comprise constant dose of a bispecific tumor transforming nanoparticle that comprises folate and SLAMF7 polypeptide attached to its surface, a constant dose of a CD47 blocking agent, and optionally, an increasing concentration of a PD1/PD-L1 blocking agent.

In some aspects, compositions are provided that comprise constant doses of a bispecific tumor transforming nanoparticle that comprises folate and SLAMF7 polypeptide attached to its surface and increasing concentrations of the CD47 blocking agent.

In some aspects, compositions are provided that comprise constant doses of a bispecific tumor transforming nanoparticle that comprises folate and SLAMF7 polypeptide attached to its surface and constant doses the CD47 blocking agent, and optionally, increasing doses of the PD1-/PD-L1 blocking agent.

In some aspects, a composition comprises an additional cancer therapy such as radiation therapy, surgery, chemotherapy, gene therapy, DNA therapy, viral therapy, RNA therapy, immunotherapy, bone marrow transplantation, nanotherapy, a further antibody therapy, or a combination of the foregoing.

### Methods of Treatment

Provided are methods of treating cancer comprising administering to a subject in need thereof a therapeutically effective amount of a particle as described herein.

In some aspects, the particle is a bispecific tumor-transforming nanoparticle. In some aspects, the method comprises administering to a subject in need thereof a therapeutically effective amount of a composition comprising a bispecific tumor-transforming nanoparticle and a CD47 blocking agent.

In some aspects, the method comprises administering to a subject in need thereof a therapeutically effective amount of a composition comprising a bispecific tumor-transforming nanoparticle and a CD47 blocking agent, and, optionally, a PD1/PD-L1 blocking agent.

In some aspects, the composition is administered through a route selected from intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrasternal, oral, rectal, topical, epidermal, mucosal, intranasal, vaginal, rectal, sublingual administration, and combinations thereof.

In some aspects, a composition comprising a bispecific tumor-transforming nanoparticle is administered through a different route than a composition comprising a CD47 blocking agent and/or a composition comprising a PD1/PD-L1 blocking agent. For example, a bispecific tumor-transforming nanoparticle is administered intratumorally and a CD47 blocking agent is administered intravenously. Or, a bispecific tumor-transforming nanoparticle is administered intratumorally and a CD47 blocking agent is administered intravenously, and a PD1/PD-L1 blocking agent is administered intramuscular.

In some aspects, the composition comprising a BiTN and the composition comprising a CD47 blocking agent and/or a P-1/PD-L1 blocking agent are administered at the same time.

In some aspects, a composition comprising a BiTN and a composition comprising a CD47 blocking agent and/or a PD1/PD-L1 blocking agent are administered sequentially. In some aspects, a composition comprising a BiTN is administered first and a composition comprising a CD47 blocking agent and/or a PD1/PD-L1 blocking agent is administered thereafter. In some aspects, a composition comprising a CD47 blocking agent and/or a PD1/PD-L1 blocking agent is administered first and a composition comprising a BiTN is administered thereafter. In some aspects, a composition comprising a CD47 blocking agent and/or a PD1/PD-L1 blocking agent is administered first and a composition comprising a BiTN is administered thereafter. In some aspects, a composition comprising a CD47 blocking agent is administered first and a composition comprising a BiTN and a PD1/PD-L1 blocking agent is administered thereafter. In some aspects, a composition comprising a PD1/PD-L1 blocking agent is administered first and a composition comprising a BiTN and a CD47 blocking agent is administered thereafter.

In some aspects, a composition described herein is administered once a day, twice a day, 3 times a day, 4 times a day, once every 2 days, once every 3 days, once every 4 days, once every 5 days, once every 6 days, once every 7 days, once every 8 days, once every 9 days, once every 10 days, once every 11 days, once every 12 days, once every 13 days, once every 14 days, once every 15 days, once every 16 days, once every 17 days, once every 18 days, once every 19 days, once every 20 days, once every 21 days, once every 22 days, once every 23 days, once every 24 days, once every 25 days, once every 26 days, once every 27 days, once every 28 days, once every 29 days, once every 30 days, once every 31 days, once a month, once every 2 months, once every 3 months, once every 4 months, once every 5 months, once every 6 months, once every 7 months, once every 8 months, once every 9 months, once every 10 months once every 11 months or once every 12 months.

The cancer cells can be breast cancer cells, brain cancer cells, prostate cancer cells, lung cancer cells, or colorectal cancer cells.

In some aspects, when administered to a subject having a tumor the composition comprising a BiTN induces lymphocyte infiltration in a tumor. In some aspects, a composition comprising a BiTN when administered to a subject having a tumor induces CD4+ lymphocyte infiltration in a tumor. In some aspect a composition comprising a BiTN when administered to a subject having a tumor induces CD8+ lymphocyte infiltration in a tumor.

In some aspects, a composition comprising a BiTN when administered to a subject having a tumor induces memory T cell activation in the subject.

In some aspects, a composition comprising a BiTN when administered to a subject having a tumor induces central memory phenotype T cells in the subject.

In some aspects, a composition comprising a BiTN when administered to a subject having a tumor induces macrophage infiltration in a tumor.

In some aspects, a composition comprising a BiTN when administered to a subject having a tumor induces dendritic cell infiltration in a tumor.

In some aspects a composition comprising a BiTN when administered to a subject having a tumor induces specific activation of macrophages in a tumor.

### EXAMPLES

### Example 1—Engineering BiTN for Solid Tumor Conversion

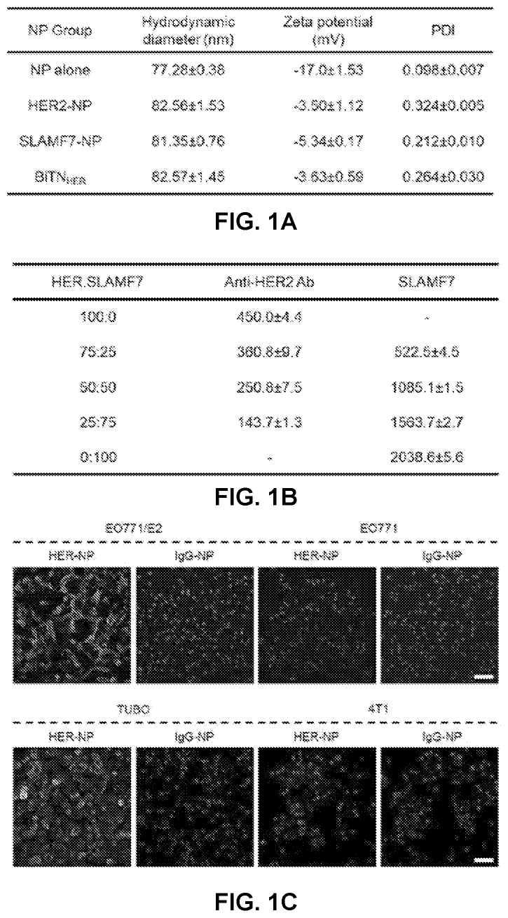

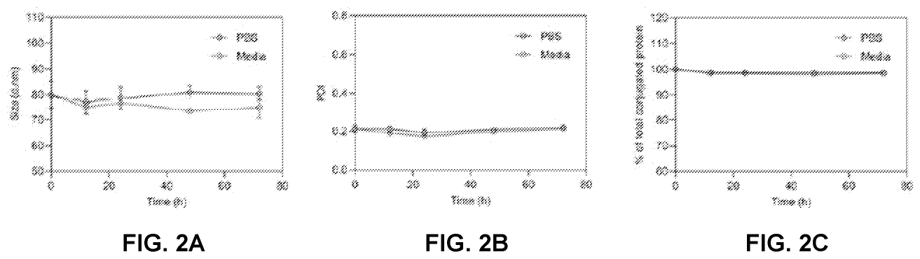

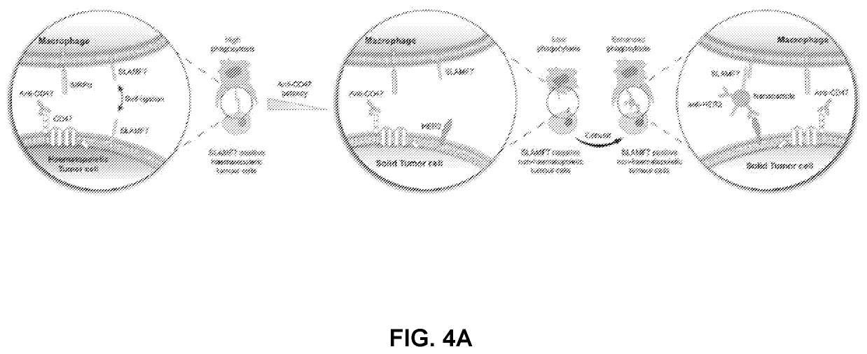

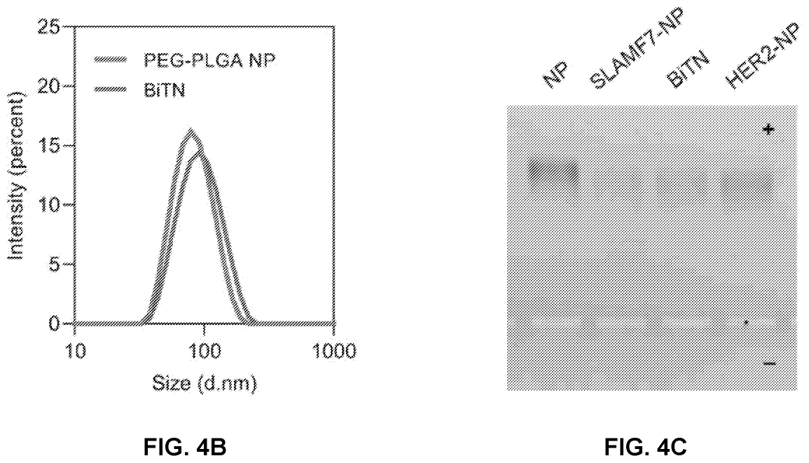

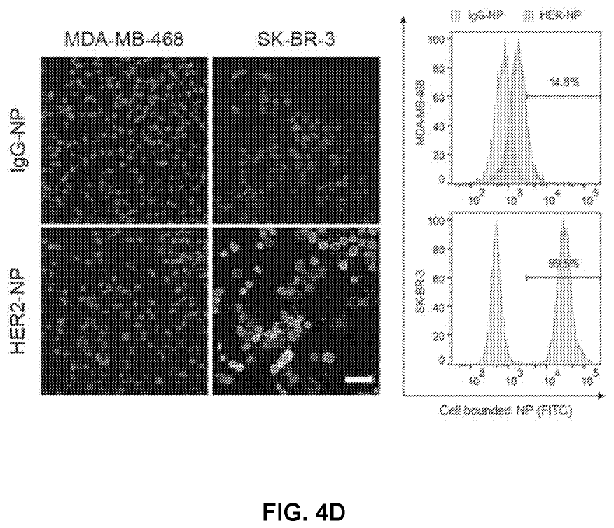

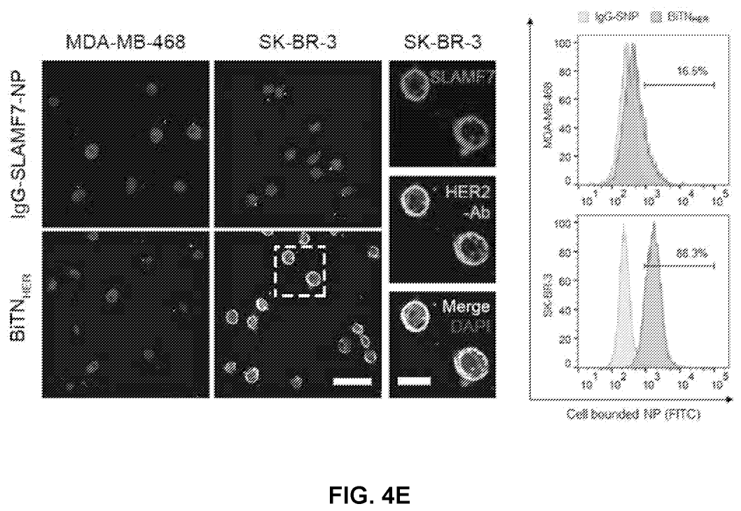

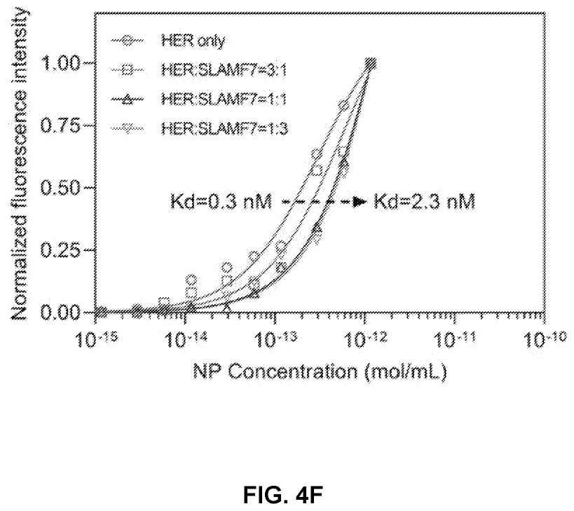

A universal nanobioconjugate platform was developed, which was termed bispecific tumor-transforming nanoparticles (BiTN). The BiTN is composed of a polymeric core with amine-reactive crosslinkers to conjugate exchangeable tumor-targeting ligands, and SLAMF7. To demonstrate that BiTN can selectively bind to solid tumor cells in a targeted manner, breast cancers that express the human epidermal growth factor receptor 2 (HER2) receptor were selected. BiTN were generated by conjugating polymeric nanoparticles with anti-HER2 antibody (Table 2) and recombinant SLAMF7 (BiTN_{HER}) (Table 1). Attaching anti-HER2 antibody and/or SLAMF7 slightly increased the size and zeta potential of the polymeric nanoparticles (NPs) (FIG. 4B and FIG. 1) but maintained their monodispersity (FIG. 4C). The increased size indicated the successful conjugation of antibody/protein onto the NP surface. The valency of conjugated antibody/SLAMF7 was calculated (FIG. 1). These BiTN nanoparticles found were to be stable in aqueous solutions with no obvious change of size, polydispersity index (PDI) and protein detachment noted within 72 hours either in PBS or FBS containing media (FIG. 2). The conjugated anti-HER2 antibody showed selective targeting to HER2-overexpressing human and mouse breast cancer cells (FIG. 4D and FIG. 1), which allowed the successful SLAMF7 labeling of HER2^{high} cells with the bispecific BiTN_{HER} (FIG. 4E). Although bispecific conjugation of SLAMF7 to the anti-HER2 antibody conjugated nanoparticles decreased the binding avidity between the NPs and HER2 receptors on the cell surface, as the dissociation constants (Ka) increased with the increased ratio of conjugated SLAMF7:anti-HER2 antibody, the binding efficiency of BiTN_{HER} became much higher than free anti-HER2 antibody owing to the multivalency of NPs (Weissleder, R., Kelly, K., Sun, E. Y., Shtatland, T. & Josephson, L. Cell-specific targeting of nanoparticles by multivalent attachment of small molecules. Nat. Biotechnol. 23, 372 1418-1423 (2005) (FIG. 4F). The dissociation constant Kd increased when the conjugation ratio of anti-HER2 antibody decreased.

### Example 2—BiTN Sensitizes Tumor Cells to CD47 Blockade

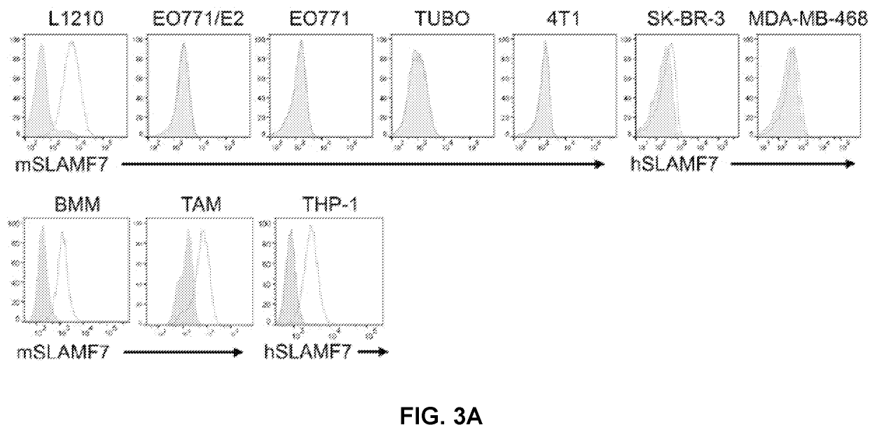

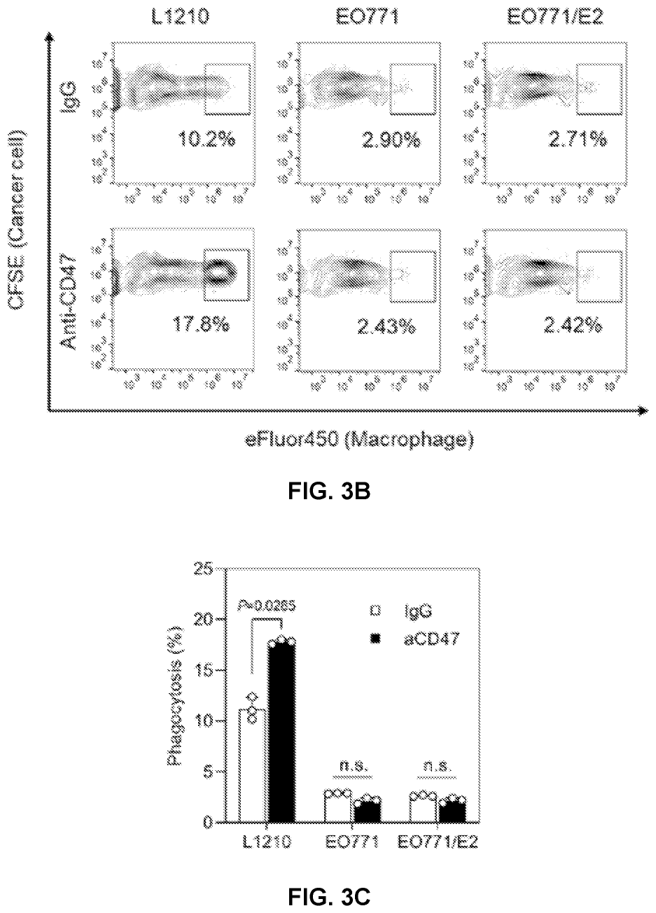

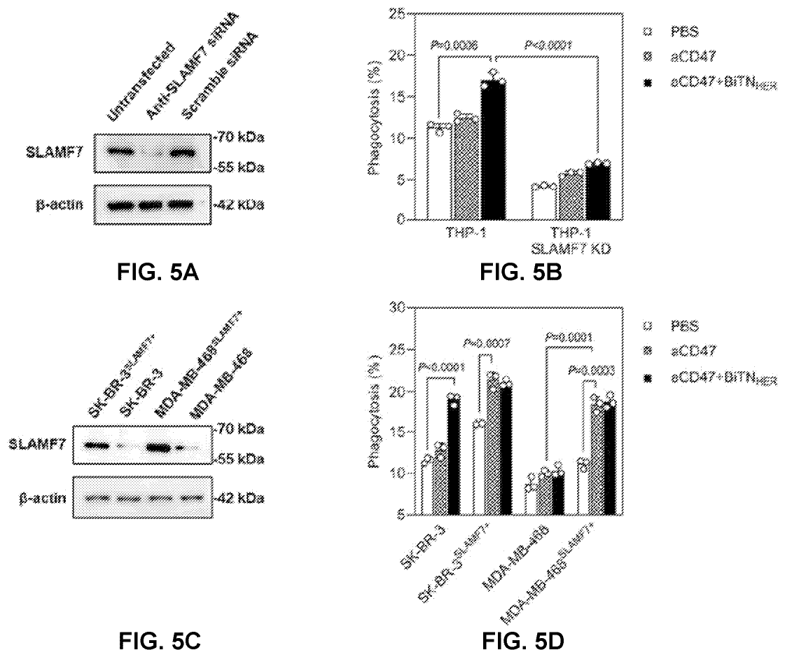

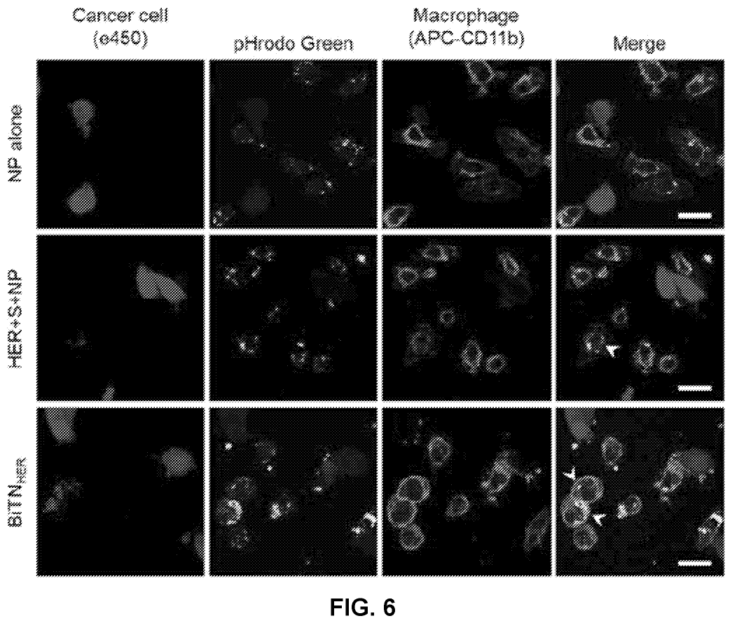

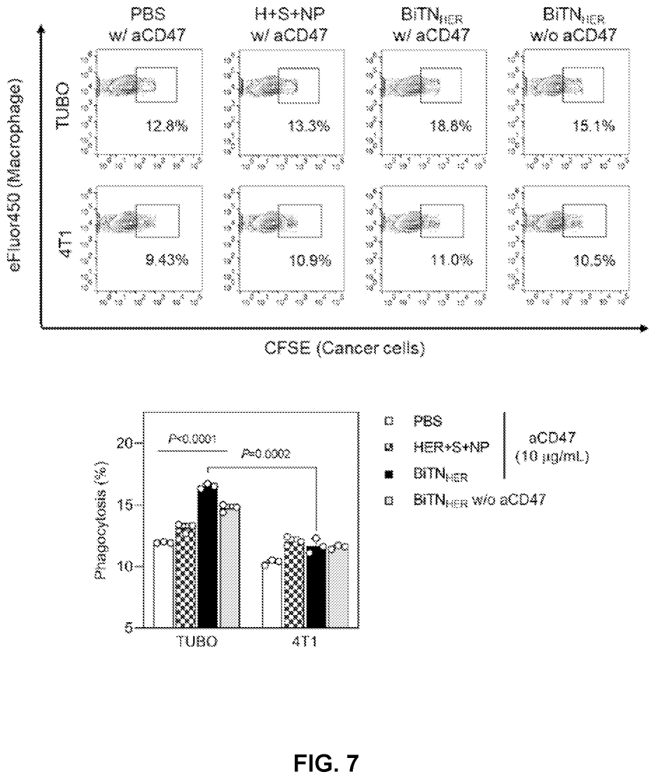

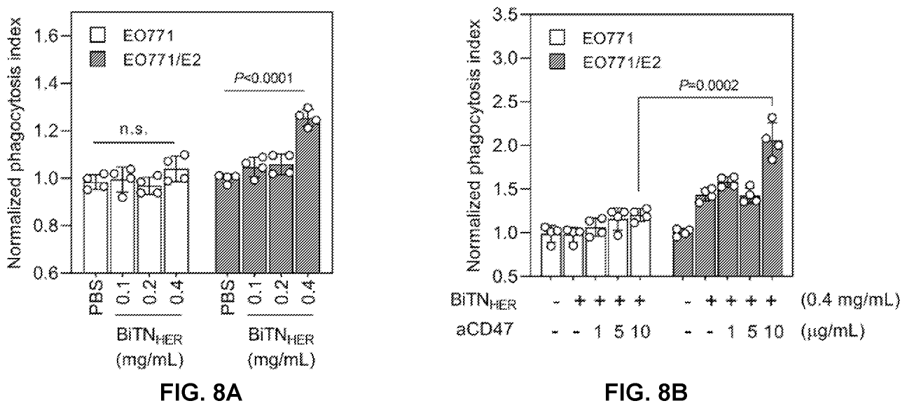

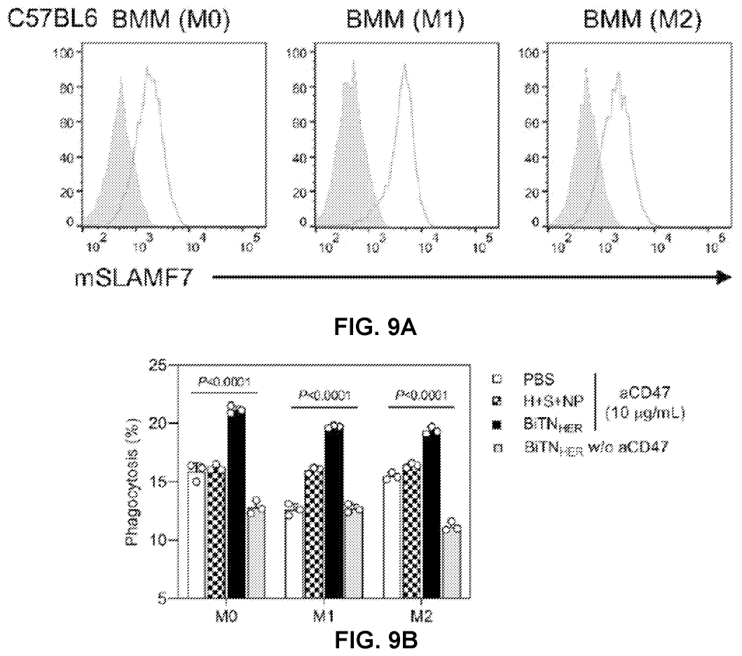

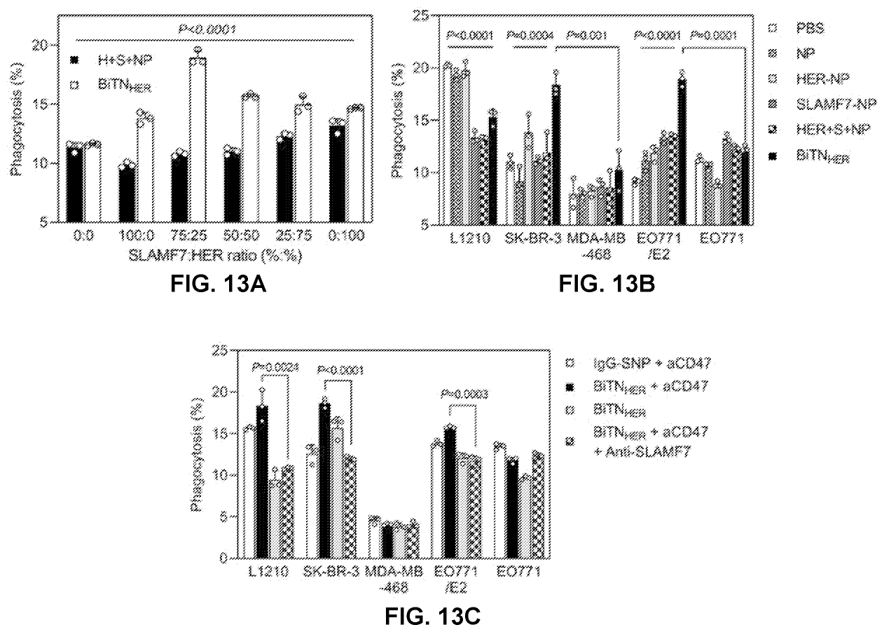

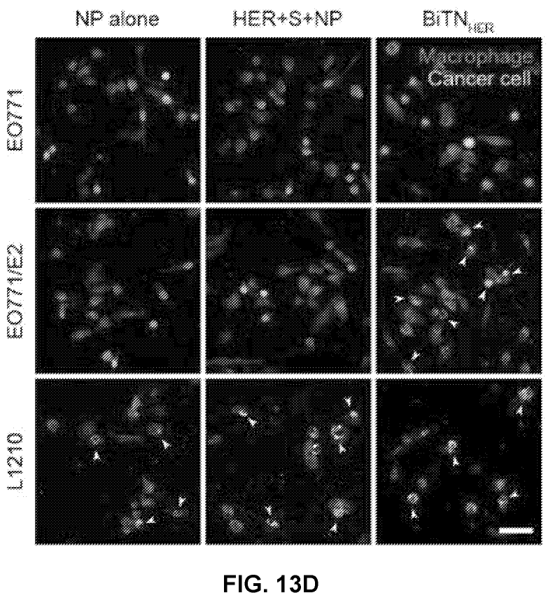

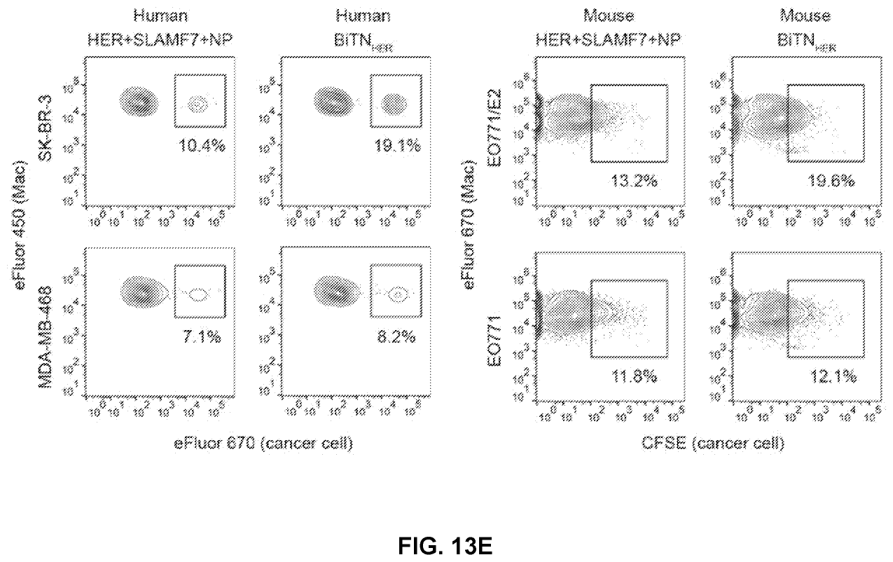

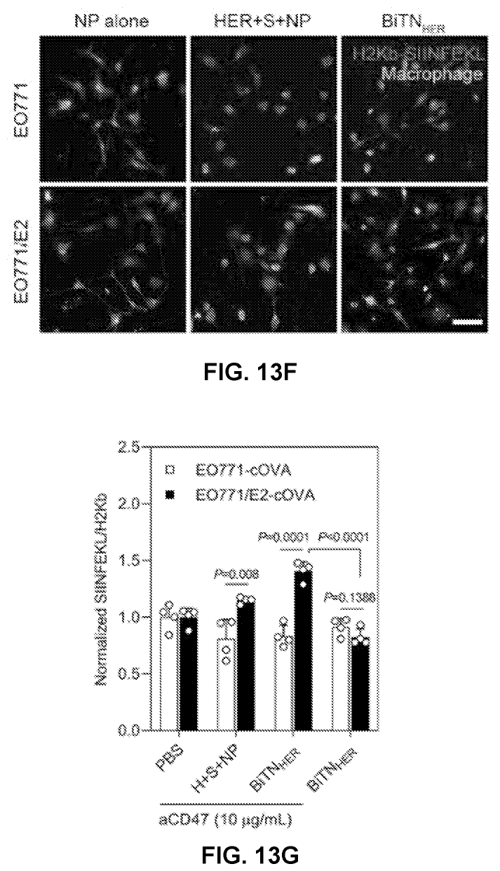

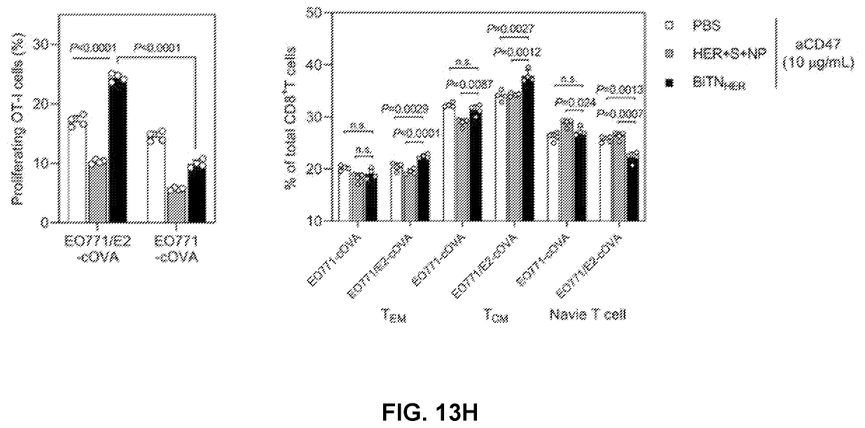

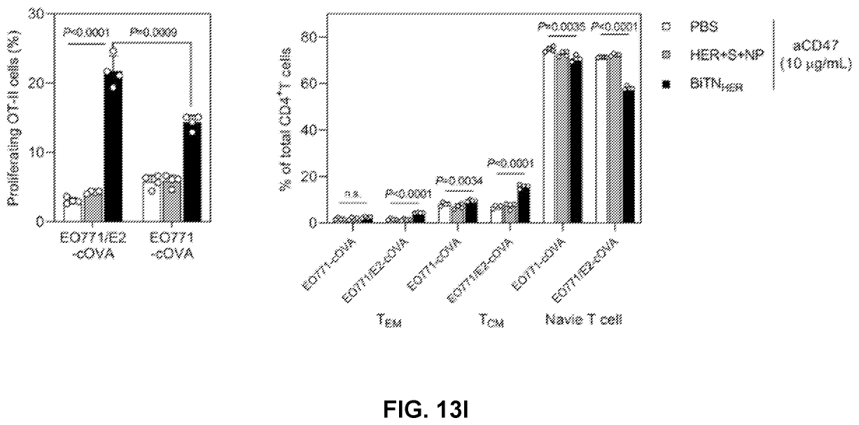

To function as an “eat me” signal and induce innate immune responses, SLAMF7 needs to be expressed on target cancer cells as a homotypic receptor and interact with the SLAMF7 on phagocytes for effective recognition and phagocytosis (Chen, J. et al. SLAMF7 is critical for phagocytosis of haematopoietic tumour cells via Mac-1 integrin. Nature 544, 493-497 (2017). For instance, SLAMF7 is exclusively expressed is on hematologic cells, thus making cancer cells of hematopoietic lineage much more sensitive to CD47 blockade-mediated macrophage phagocytosis (FIG. 3). To adopt SLAMF7-mediated sensitization of CD47 blockade in solid tumors using BiTN, target cells must first have optimal BiTN binding via receptor-targeting. By varying the ratio of conjugated SLAMF7:anti-HER2 antibody on the NPs, we determined that a 3:1 ratio induced the most robust degree of macrophage phagocytosis of HER2^{high} SK-BR-3 human breast cancer cells compared with unconjugated NPs (FIG. 13A). Furthermore, this pro-phagocytic effect required HER2 expression by the target cells, as increased phagocytosis by macrophages was not observed in HER2^{low} MDA-MB-468 human breast cancer cells (FIG. 13B). However, HER2 expression was not necessary if SLAMF7 was sufficiently abundant on target cancer cells. Indeed, a high degree of phagocytosis was observed in SLAMF7^{high}HER2^{low} L1210 cells regardless of NP treatment. Inhibition of SLAMF7 ligation between macrophages and cancer cells by anti-SLAMF7 antibody eliminated the pro-phagocytic effect of BiTN_{HER} (FIG. 13C). To further substantiate the role of SLAMF7 in promoting macrophage phagocytosis, we generated SLAMF7 overexpressed human breast cancer cell line SK-BR-3^{SLAMF7+} and MDA-MB-468^{SLAMF7+} as well as SLAMF7 knock-down in human macrophage THP-1^{SLAMF7-} (FIG. 5). In contrast to parental cells, both SLAMF7 overexpressing cancer cells exhibited increased phagocytosis by macrophages in response to CD47 blockade. On the other hand, SLAMF7 knock-down in THP-1 cells significantly abrogated BiTN-induced macrophage phagocytosis of HER2^{high} SK-BR-3 cells. Together, these results indicated that the targeted transformation of HER2^{high} breast cancer cells to a SLAMF7^{high} phenotype is essential to initiate phagocytosis by macrophages. Moreover, changing the conjugation of human anti-HER2 antibody to a mouse anti-HER2/neu antibody led to antigen-specific, SLAMF7-mediated phagocytosis in HER2/neu overexpressing EO771/E2 and TUBO mouse breast cancer cells (FIG. 13D, E and FIG. 6-7). The BiTN_{HER} itself could induce moderate macrophage phagocytosis at high concentrations, but it greatly sensitized the HER2^{high} EO771/E2 cells to CD47 blockade as an adjuvant, since the phagocytic activity was further elevated when BiTN_{HER} was combined with anti-CD47 antibody (aCD47) treatment (FIG. 8). Given that macrophages are often polarized in the tumor microenvironment, which affects their phagocytic ability (Gordon, S. R. et al. PD-1 expression by tumour-associated macrophages inhibits phagocytosis and tumour immunity. Nature 545, 495-499 (2017)), we further tested the pro-phagocytic effect of the BiTN_{HER} and aCD47 combination on M1 macrophages polarized by lipopolysaccharide (LPS) and on M2 macrophages polarized by interleukin-4 (IL-4), the latter phenotypically more resemble that of tumor-associated macrophages. The polarization of macrophages had little impact on the expression level of SLAMF7 on the surface of the macrophages, or on activation of phagocytosis against EO771/E2 cells mediated by BiTN_{HER} and aCD47 (FIG. 9).

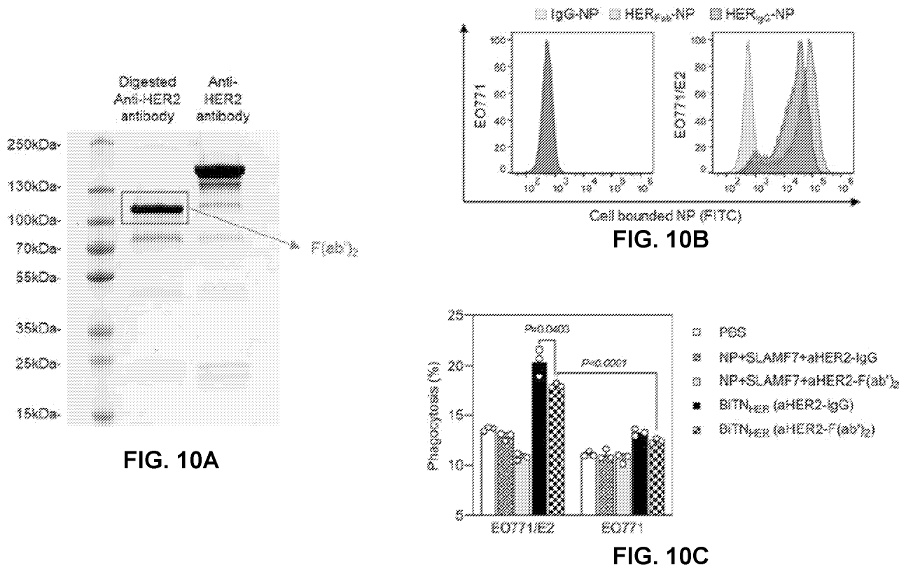

Since monoclonal antibody itself could induce antibody-dependent cellular phagocytosis (ADCP) through engagement of Fc receptor on macrophage, we generated Fc silenced anti-HER2 antibody to rule out the possibility that the Fc fragments on BiTN were responsible for promoting phagocytosis of tumor cells (FIG. 10). Fc fragment was digested with pepsin and F(ab′)2 fragment of anti-HER2 antibody was obtained with similar HER2 targeting ability as whole anti-HER2 antibody. We prepared the bispecific nanoconjugate with F(ab′)2 fragment (designated as BiTN_{HER}(aHER2-F(ab′)2)) and with whole IgG antibody (designated as BiTN_{HER}(aHER2-IgG)). BiTN_{HER}(aHER2-F(ab′)2) treated EO771/E2 had slightly lower phagocytosis by macrophage compared with BiTN_{HER}(aHER2-IgG) group, but the Fc silenced NPs still showed potent and selective pro-phagocytosis effects against HER2-positive EO772/E2 cells (FIG. 10). These results demonstrate that BiTN-mediated macrophage phagocytosis is only partially induced by the Fc engagement from the conjugated antibody, the major pro-phagocytosis effect is mediated through the SLAMF7 labeling of the HER2-positive cancer cell and engaging with SLAMF7 on macrophages.

As a professional APC, macrophages phagocytize cancer cells, present tumor-derived antigens and prime T lymphocytes (Yuan, H. et al. Multivalent bi-specific nanobioconjugate engager for targeted cancer immunotherapy. Nat. Nanotechnol. 12, 763-769 (2017)). To test whether the combination of BiTN_{HER} and aCD47 could bridge these innate and adaptive immune responses, we co-cultured EO771/E2 and EO771 mouse breast cancer cells expressing cytoplasmic ovalbumin (cOVA) with macrophages and exposed these macrophages with OVA-specific CD4⁺ (OT-II) and CD8⁺ (OT-I) T cells. Consistent with the macrophage-phagocytosis findings, enhanced presentation of OVA peptide with major histocompatibility complex I (the SIINFEKL-H2kb complex) was observed only on macrophages co-cultured with BiTN_{HER} and aCD47 co-cultured with HER2^{high} EO771/E2-cOVA cells (FIG. 13F). The combination treatment induced higher levels of antigen presentation compared with unconjugated NPs, and the selectivity over HER2^{high} cancer cells was abrogated when aCD47 was absent (FIG. 13G). We next evaluated the priming of OVA-specific T cells after exposure to APCs. Similarly, the combination treatment led to greater proliferation of both CD4⁺ and CD8⁺ T cells from the co-culture system of macrophages and EO771/E2-cOVA cells (FIG. 13H, I). Within the activated CD8+ T cells, we found a more significant phenotypic shift from naïve T cells towards effector memory (TEM) and central memory (TCM) T cells in the BiTN_{HER}- and aCD47-treated group compared with aCD47-alone or aCD47- and unconjugated NPs-treated groups (FIG. 13H). Although minor shifting was noted in CD4+ T cells, the overall activation level was much less than in CD8+ T cells (FIG. 13I).

### Example 3—BiTN Enhances Antitumor Effect of CD47 Blockade

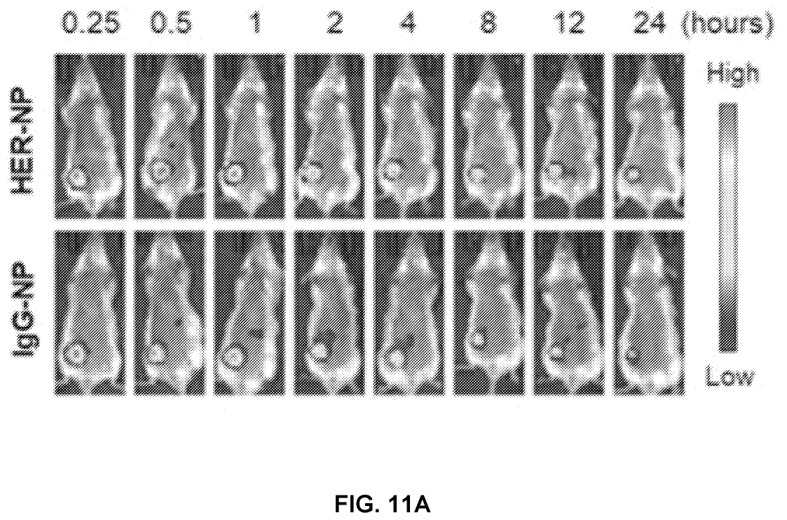

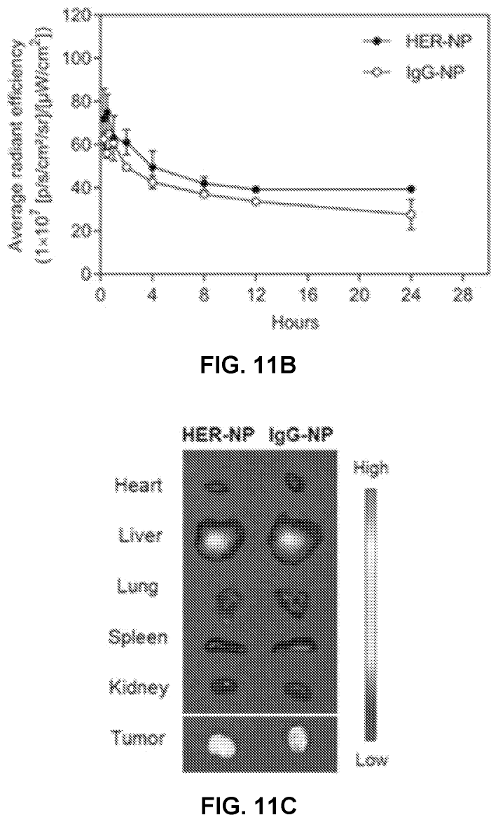

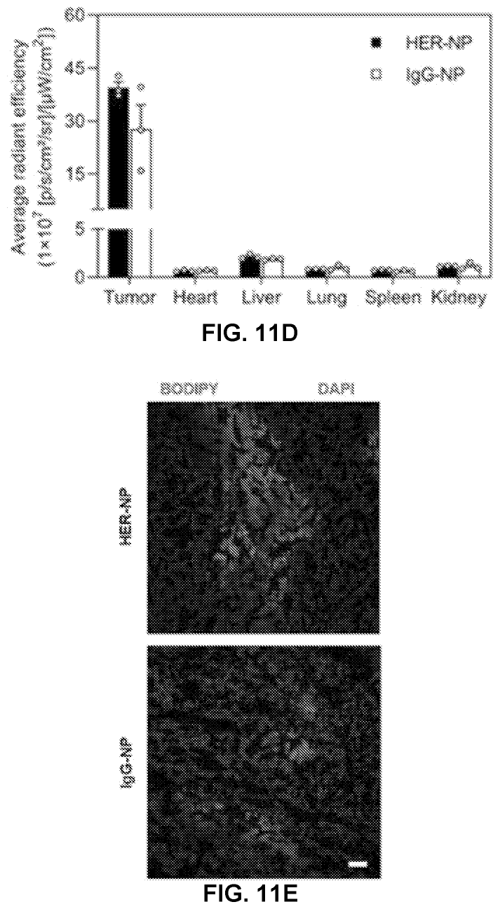

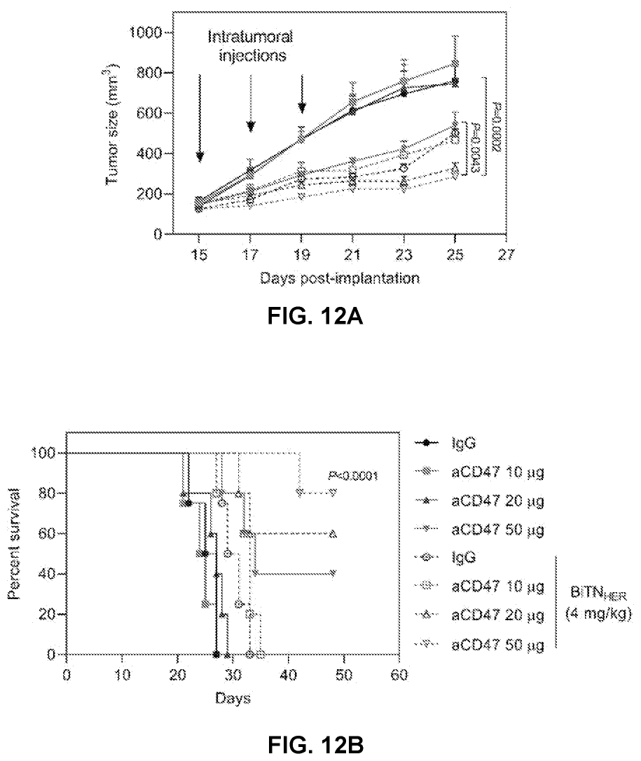

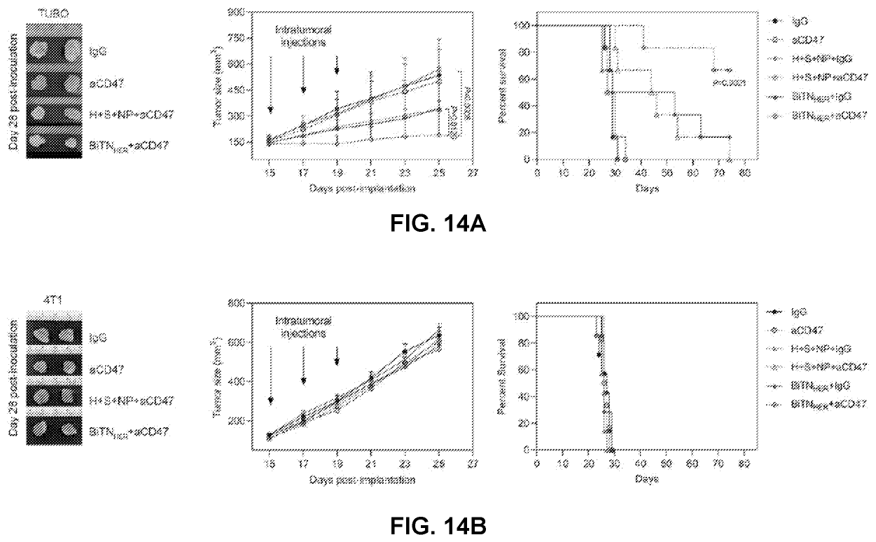

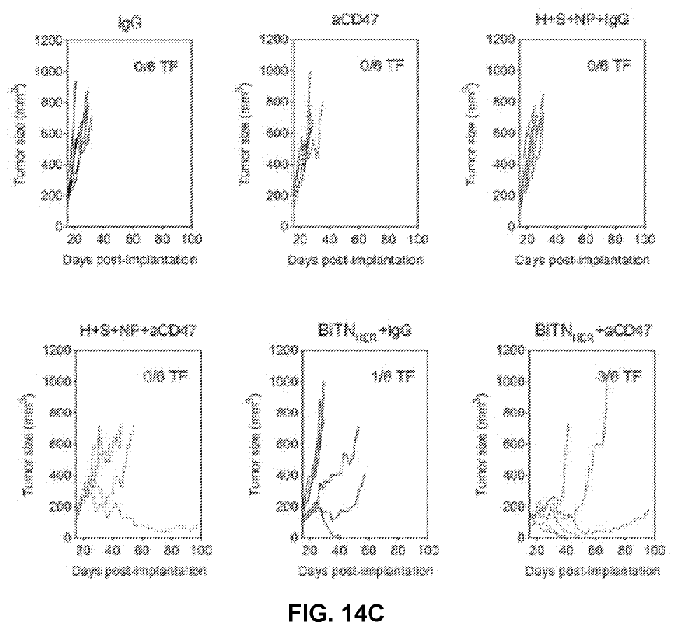

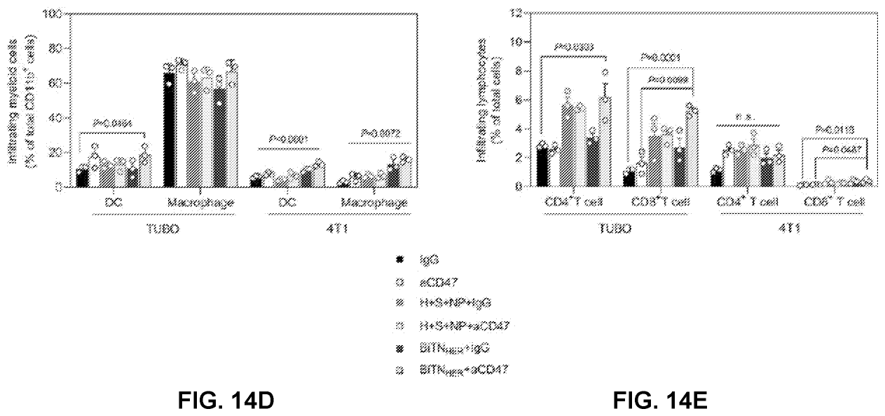

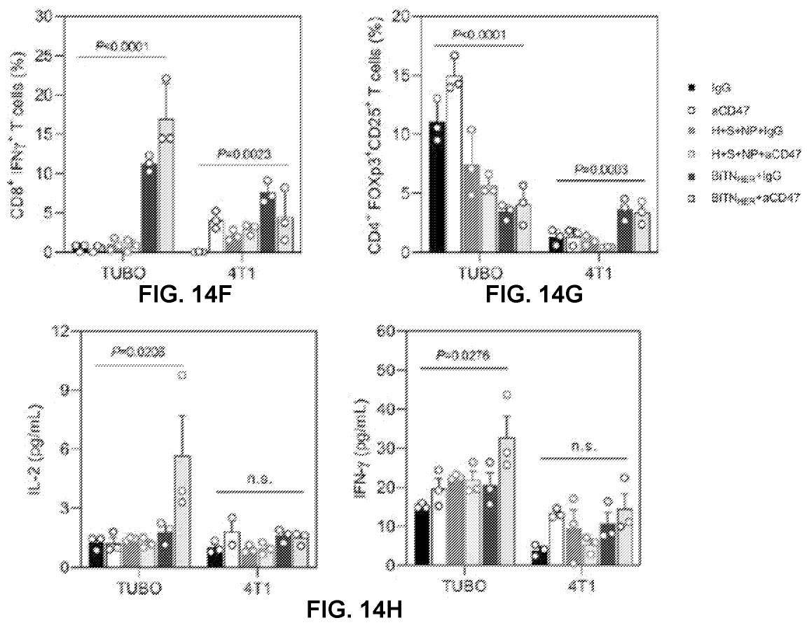

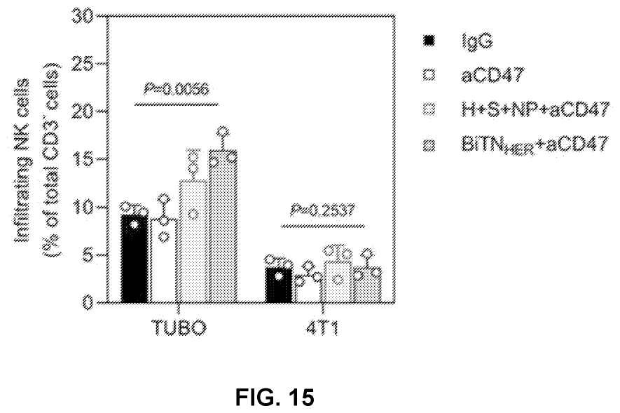

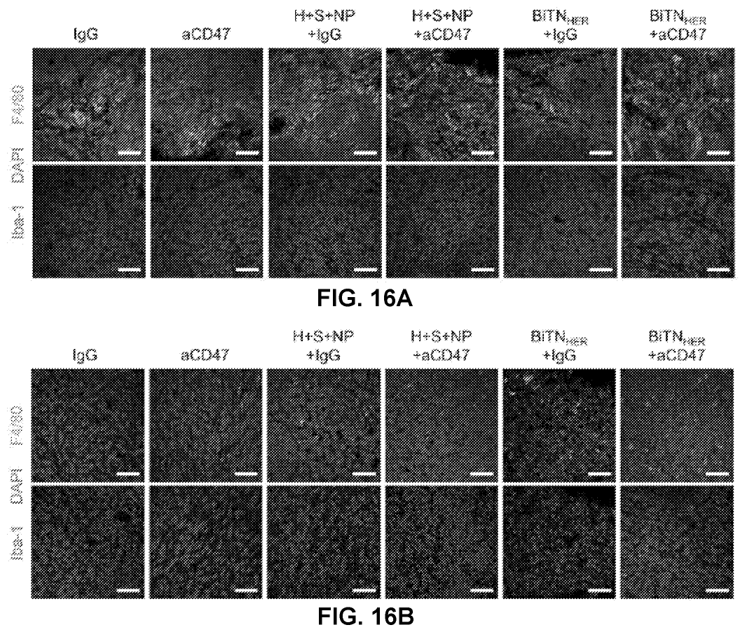

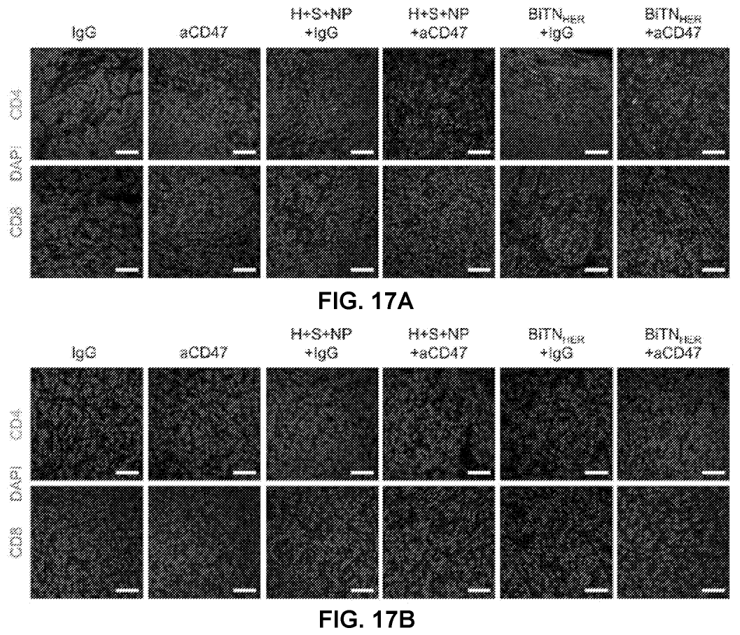

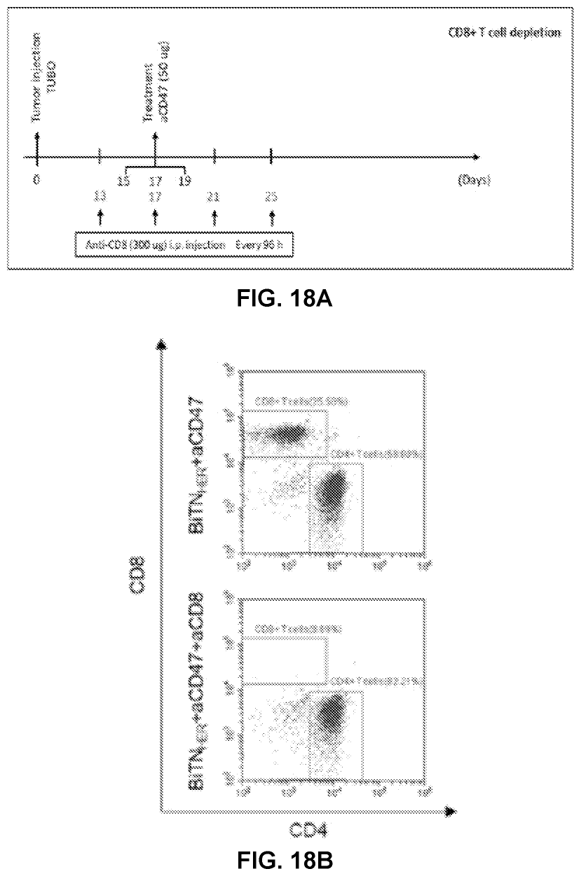

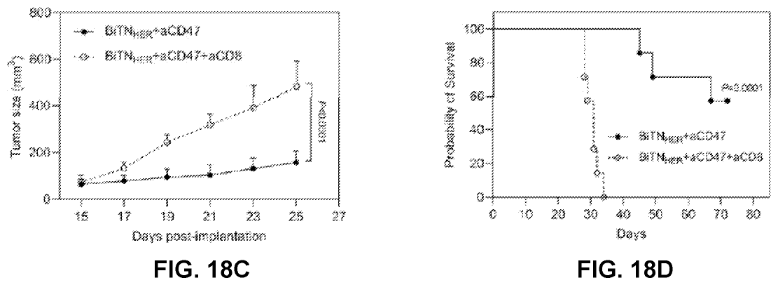

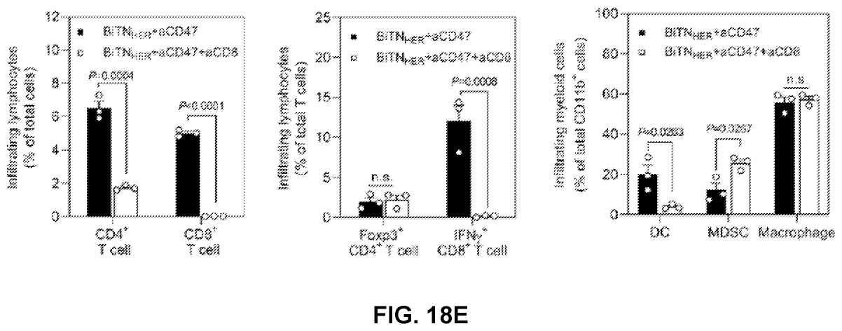

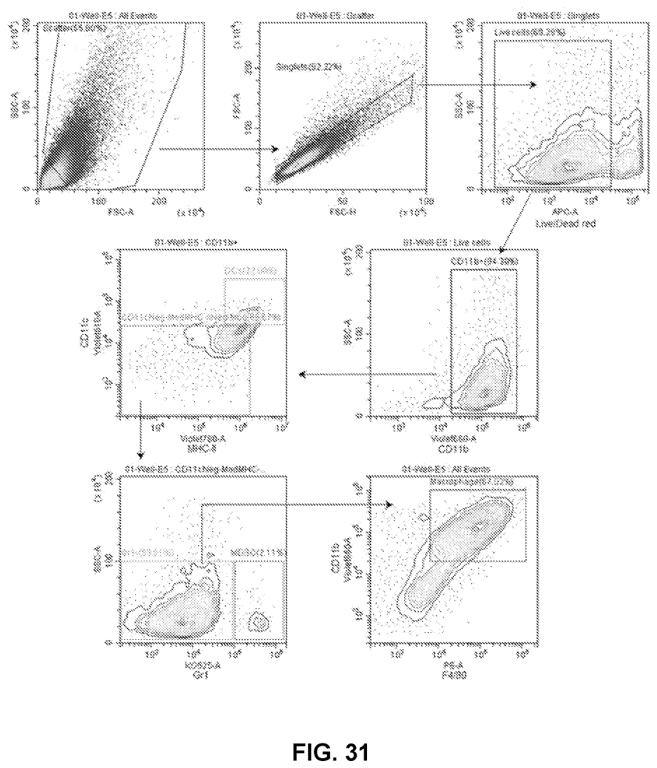

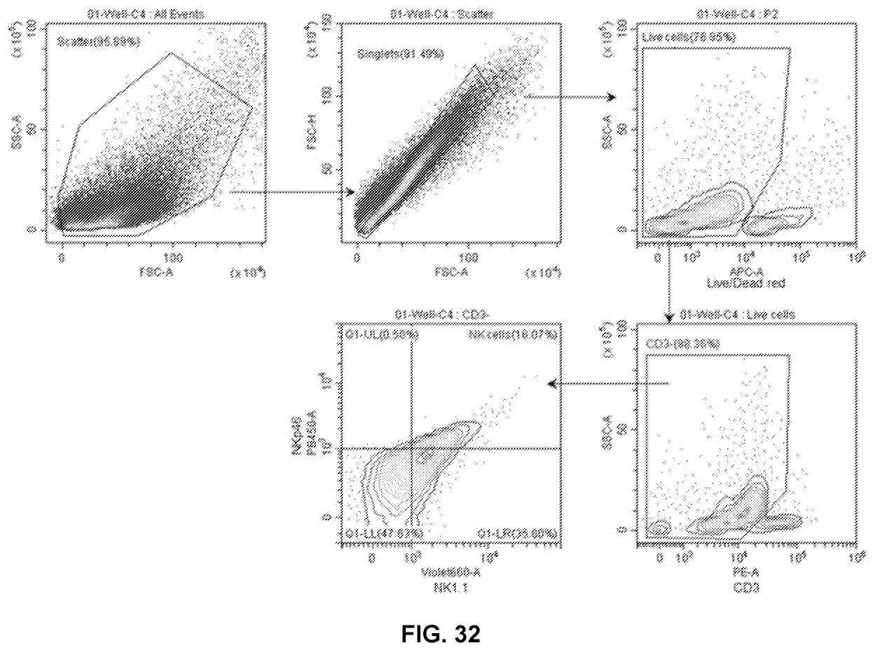

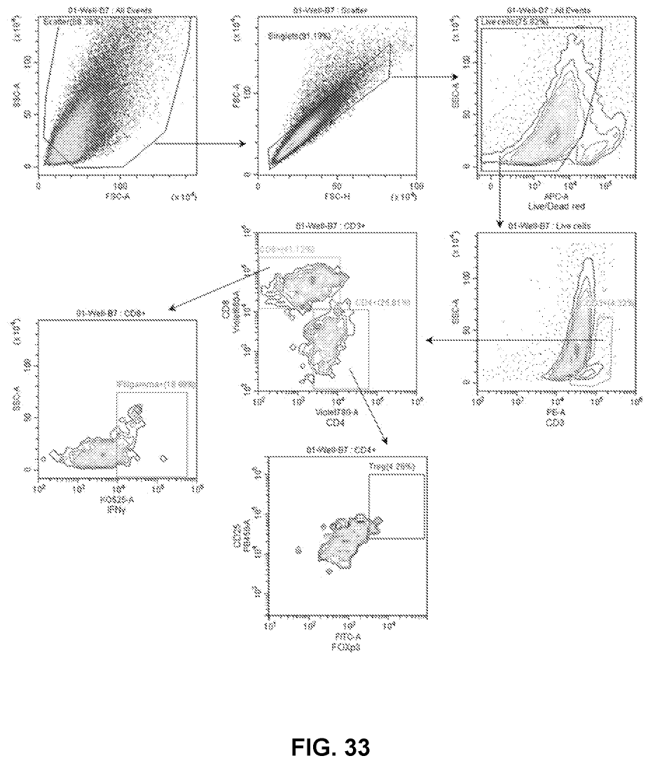

Given the strong ability of BiTN_{HER} and aCD47 combination therapy to initiate immune responses in vitro, we next evaluated its antitumor potential in orthotopic mouse breast tumor models by using TUBO breast cancer cells, which express the rodent homolog of the HER2 protein, and 4T1 cells, which lack the receptor. Intratumoral injections of BiTNs resulted in a long retention within the tumor with minimal nanoparticle distribution in peripheral organs (FIG. 11). A combination of BiTN_{HER} and aCD47 at a predetermined dosage led to significant tumor inhibition compared with monotherapy with BiTN_{HER} or aCD47 in neu^{high} TUBO tumors (FIG. 14A and FIG. 12). The combination treatment significantly reduced tumor burden and prolonged the survival of TUBO tumor-bearing mice, with 3 of 6 mice remaining tumor free (FIG. 14C). However, the same antitumor effect was not observed in neu^{low} 4T1 tumor-bearing mice (FIG. 14B). To elucidate the tumor-specific inhibitory effect of the combination therapy, we first examined the tumor infiltration of both innate and adaptive immune cells. Treatment with BiTN_{HER} and aCD47 led to increased infiltration of natural killer (NK) cells and DCs but minimal change to the overall number of macrophages in TUBO tumors (FIG. 14D and FIG. 15). However, upon immunostaining ionized calcium binding adaptor molecule 1 (Iba-1), a macrophage-specific calcium-binding protein often used as a marker of macrophage activation, we found the highest levels of activated macrophages in the treated TUBO tumors (FIG. 16). Treatment with BiTN_{HER} and aCD47 significantly increased the overall number of T cells in TUBO but not in 4T1 tumors (FIG. 14E and FIG. 17). Interestingly, lower percentages of immunosuppressive CD4+ regulatory T cells (Tregs) and higher percentages of interferon γ (IFN-γ)-secreting CD8+ effector T cells were found in TUBO tumors after the combination treatment, relative to untreated TUBO tumors or treated 4T1 tumors (FIG. 14F, G). Moreover, depletion of CD8+ T cells completely abolished the antitumor effect of BiTN_{HER} and aCD47 combination therapy (FIG. 18). This removal of cytotoxic IFN-γ+CD8+ T cells from the tumor microenvironment led to significantly lower infiltration of DCs and more immunosuppressive myeloid-derived suppressor cells (MDSCs) (FIG. 18). These results suggest that BiTN_{HER} transformed HER2/neu^{high} tumors to SLAMF7^{high} tumors, thereby promoting tumor-specific activation of local innate immune responses, as we observed previously in vitro. These results further support the finding that SLAMF7 ligation physically triggers macrophage activation and synergistically promote CD47 blockade to bridge the innate and adaptive immune systems and enhance the elimination of tumor cells (Cao, X. et al. Effect of cabazitaxel on macrophages improves CD47-targeted immunotherapy for triple-negative breast cancer. J. Immunother. Cancer 9 (2021); Pazina, T. et al. Enhanced SLAMF7 Homotypic Interactions by Elotuzumab Improves 376 NK Cell Killing of Multiple Myeloma. Cancer Immunol. Res. 7, 1633-1646 (2019)). FIGS. 31-32 show the gating strategy used for analyzing myeloid cells, including dendritic cells (DCs), myeloid-derived suppressor cells (MDSCs), macrophages and T cells from CD45-selected cells from tumors. FIG. 33 shows the gating strategy for analyzing NK cells from CD45-selected cells from tumors.

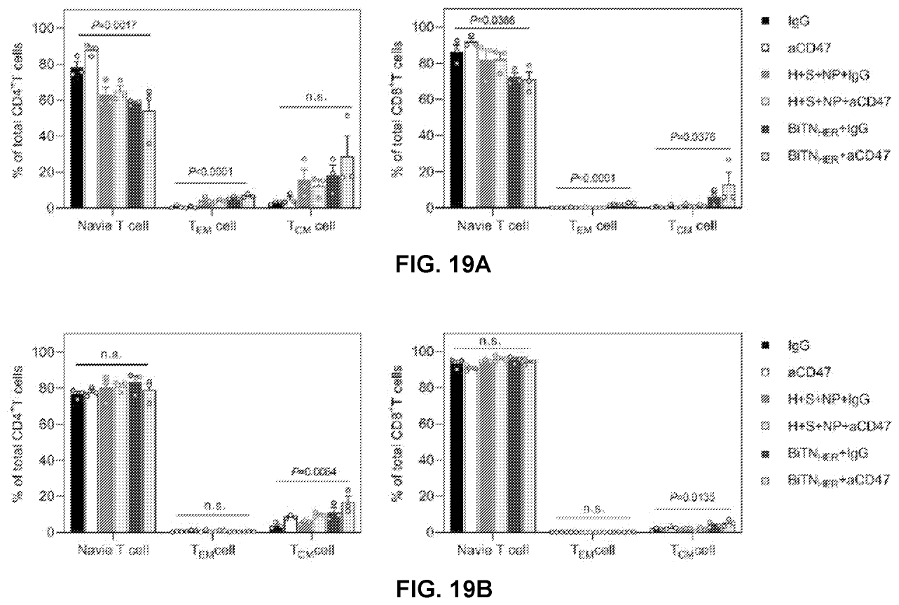

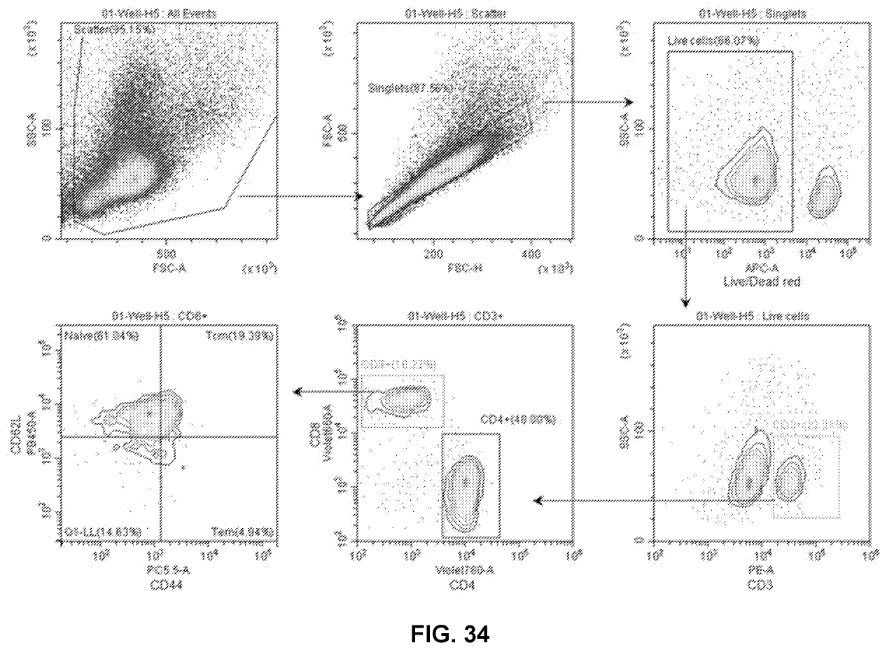

Locally applied immunotherapies have been reported to induce systemic immune responses due to the so-called cancer-immunity cycle (Shi, Y. & Lammers, T. Combining Nanomedicine and Immunotherapy. Acc. Chem. Res. 367 52, 1543-1554 (2019); Lu, K. et al. Low-dose X-ray radiotherapy-radiodynamic therapy via nanoscale metal organic frameworks enhances checkpoint blockade immunotherapy. Nat. Biomed. Eng. 2, 600-610 (2018); Shae, D. et al. Endosomolytic polymersomes increase the activity of cyclic dinucleotide STING agonists to enhance cancer immunotherapy. Nat. Nanotechnol. 14, 269-278 (2019); Li, X. et al. Cancer immunotherapy based on image-guided STING activation by nucleotide nanocomplex-decorated ultrasound microbubbles. Nat. Nanotechnol. (2022)). The cancer-immunity cycle here began with the macrophage phagocytosis mediated by BiTN. The antigen presenting cells then released and presented tumor antigens to naïve T cells, educated them into tumor-killing T cells and activated the adaptive immune response in secondary lymphatic organs like spleen. To assess systemic immune activation, we next tested whether the peripheral blood levels of the cytokines interleukin-2 (IL-2) and IFN-7 were elevated, as both are required for a proper adaptive immune (Liao, W., Lin, J. X. & Leonard, W. J. Interleukin-2 at the crossroads of effector responses, tolerance, and immunotherapy. Immunity 38, 13-25 (2013); Morad, G., Helmink, B. A., Sharma, P. & Wargo, J. A. Hallmarks of response, resistance, and toxicity to immune checkpoint blockade. Cell 184, 5309-5337 (2021); Alizadeh, D. et al. IFNgamma Is Critical for CAR T Cell-Mediated Myeloid Activation and Induction of Endogenous Immunity. Cancer Discov. 11, 2248-2265 (2021); Pitter, 391 M. R. & Zou, W. Uncovering the Immunoregulatory Function and Therapeutic Potential of the PD-1/PD-L1 Axis in Cancer. Cancer Research 81, 5141-5143 (2021)). As expected, significant increases in the cytokines were observed in BiTN_{HER}- and aCD47-treated TUBO tumor-bearing mice (FIG. 14H). Consistent with this result, the combination treatment led to decreased populations of naïve T cells in the spleens of TUBO-bearing mice; likewise, a significant shift to both the TEM and TCM phenotypes were noted relative to mice bearing TUBO tumors, compared with those bearing 4T1 tumors (FIG. 19). This selectivity of systemic immunity over the extent of HER2 expression in tumors also reveals a correlation between immunogenicity and the targeted transformation of HER2^{high} tumor to SLAMF7^{high} tumor by BiTN_{HER} treatment. FIG. 34 shows the gating strategy for analyzing splenocytes from spleens.

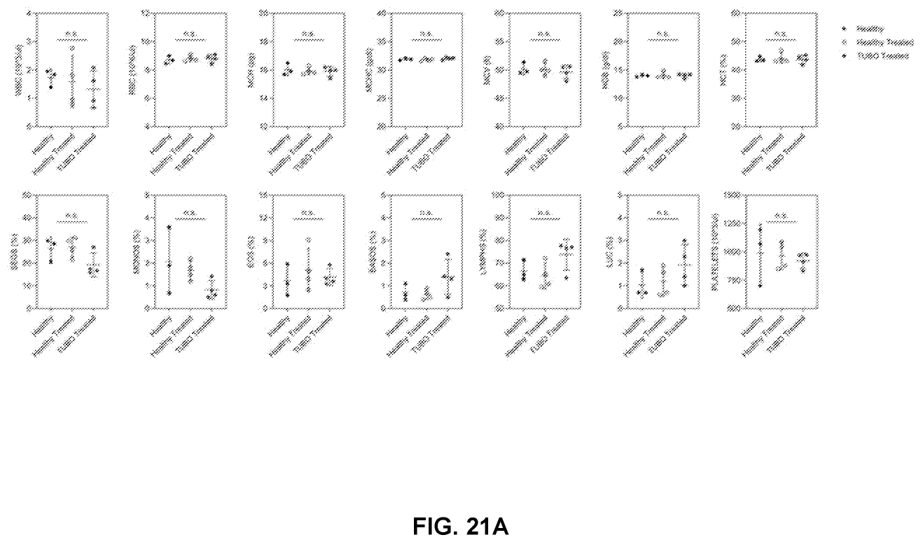

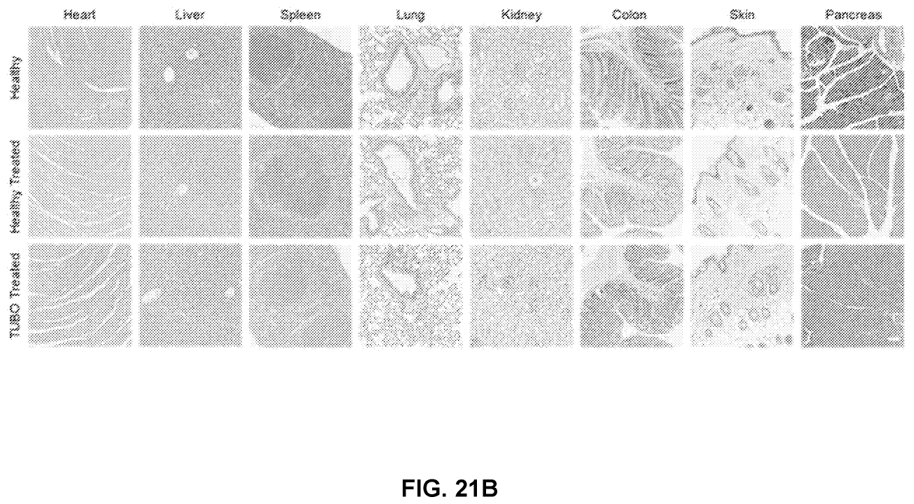

To ensure the systemic immune activation by BiTNs does not lead to autoimmune toxicity, we performed long-term toxicity study (FIG. 21). No significant change of blood counts between untreated and treated mice was found. The tumor-bearing mice had slight but not significant increases in the number of lymphocytes after treatment, which is in line with the tumor inhibition effects showing activated systemic immune responses. In addition, no obvious inflammation or lymphocyte infiltrates was found in major organs. These results support the safety profile of BiTN treatment.

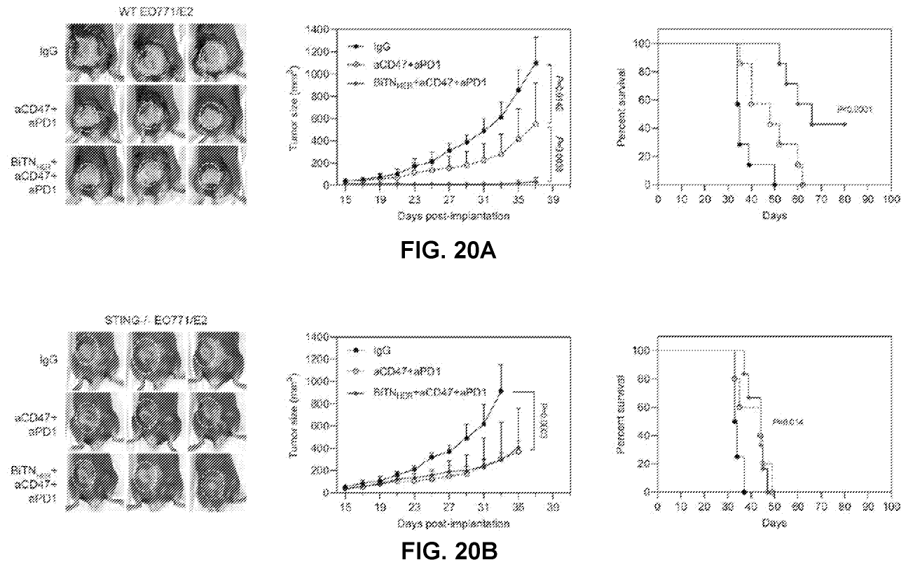

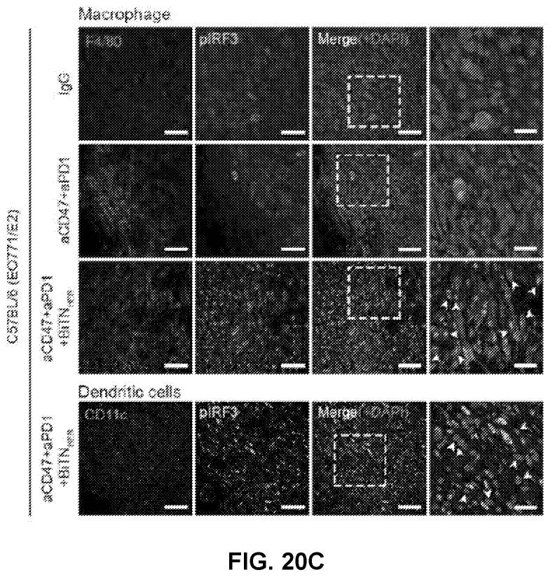

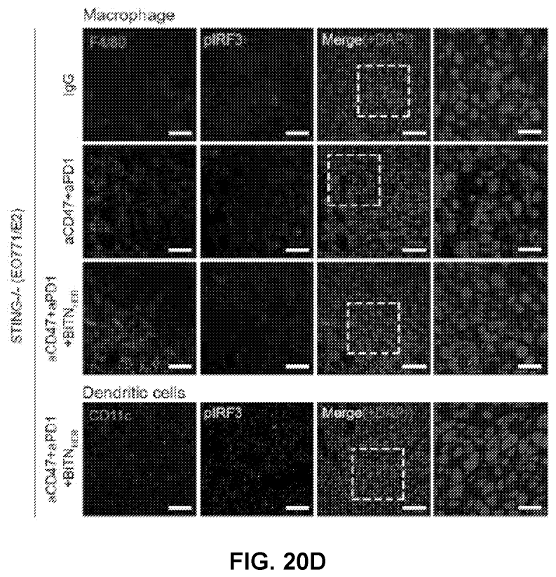

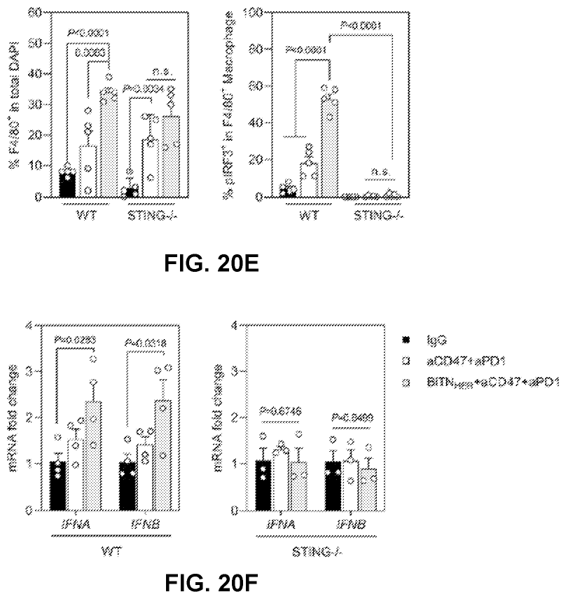

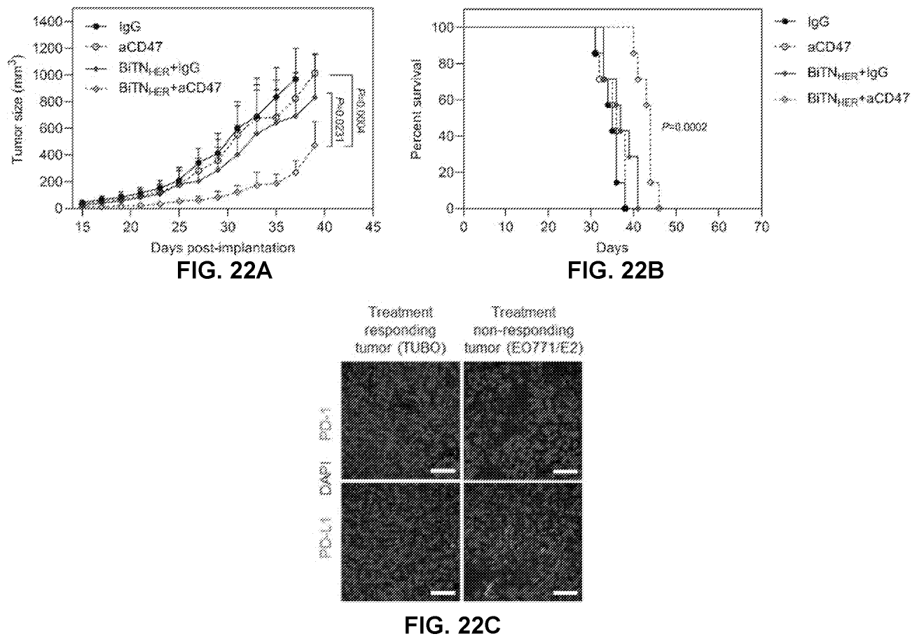

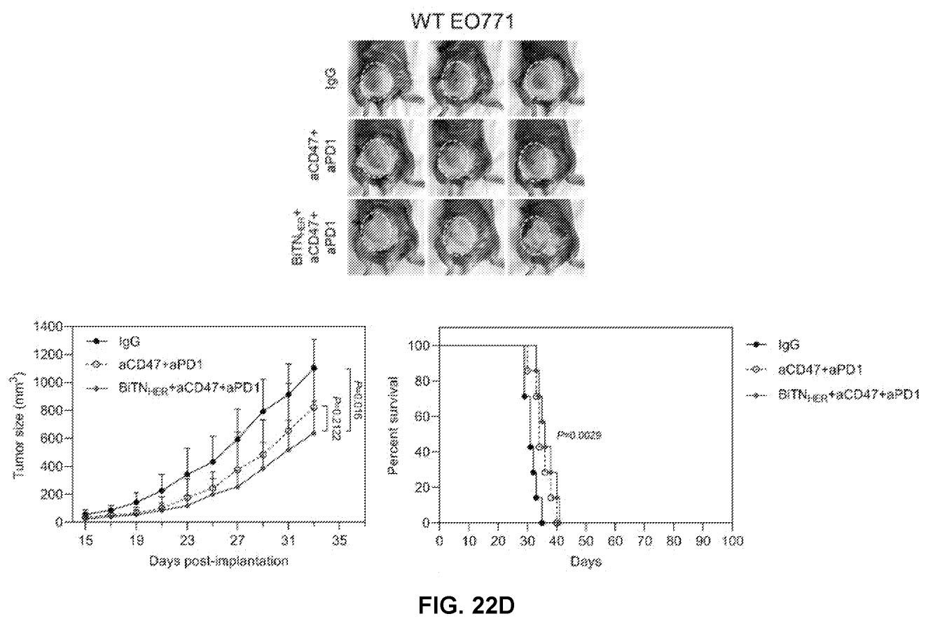

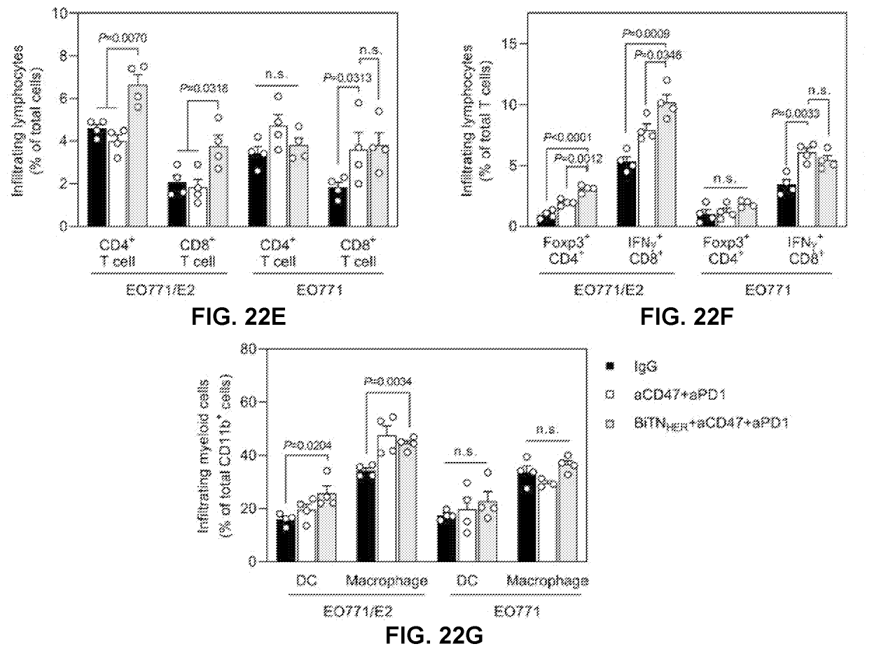

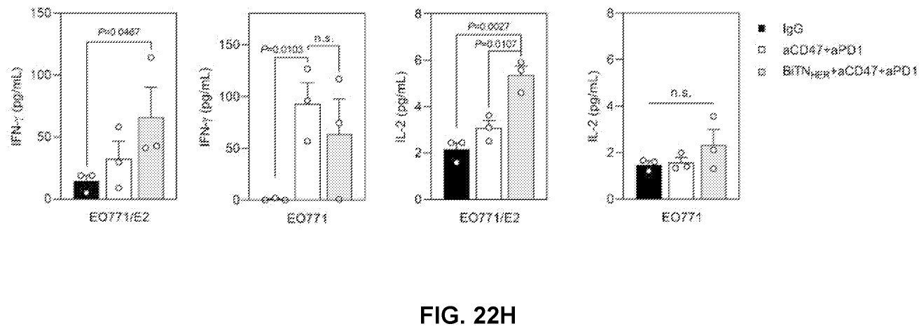

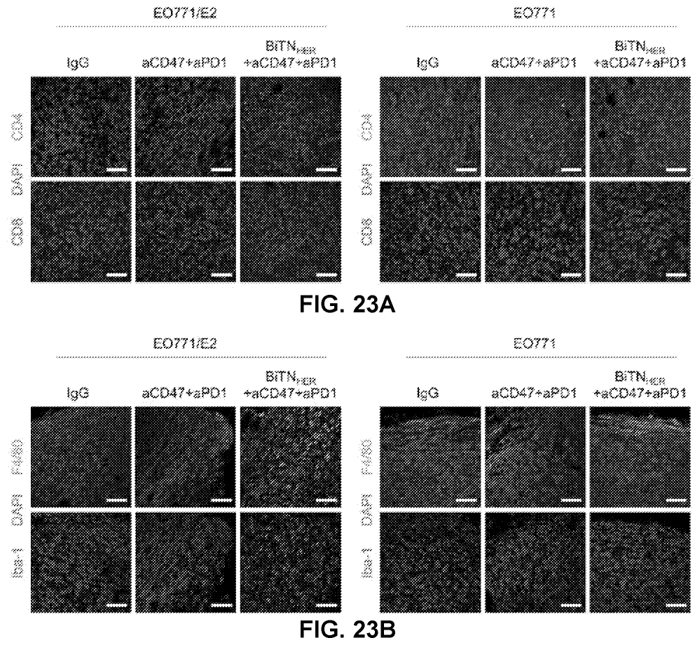

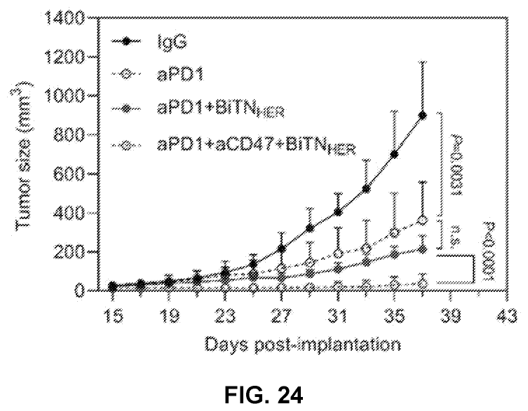

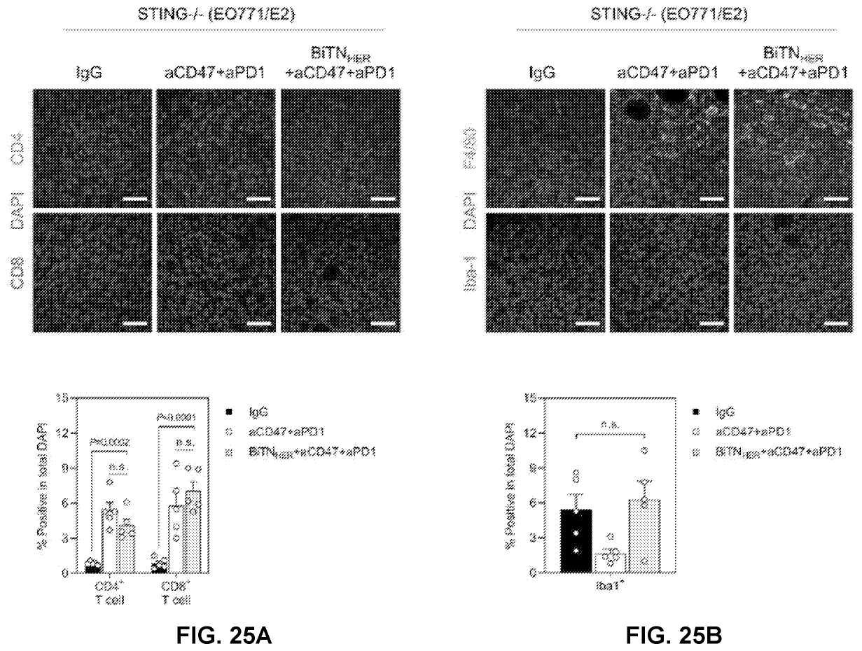

The therapeutic effects of the BiTN_{HER} and aCD47 combination were further investigated with HER2^{high} EO771/E2 and HER2^{low} EO771 tumors established in C57BL/6 mice. Unexpectedly, the antitumor effects against the EO771/E2 tumors were not as potent as those against TUBO tumors. Although tumor growth was retarded by the combination treatment compared with untreated or single-component-treated groups, all mice reached the tumor burden end point within 45 days after tumor inoculation (FIG. 22). In line with these results, we found a much higher expression of programmed death protein 1 (PD1) and programmed death-ligand 1 (PDL1) in EO771/E2 tumors than on treatment-responding TUBO tumors, suggesting a more immunosuppressive tumor microenvironment²⁷⁻²⁹. Addition of an anti-PD1 antibody (aPD1) greatly enhanced the antitumor effect of BiTN_{HER} with greater tumor growth inhibition (FIG. 20A). Similar to the results in TUBO tumors, the triple combination of aPD1, BiTN_{HER}, and aCD47 induced greater local infiltration of tumor-killing immune cells and enhanced systemic immune responses (FIGS. 22-23). FIGS. 31-32 show the gating strategy used for analyzing myeloid cells, including dendritic cells (DCs), myeloid-derived suppressor cells (MDSCs), macrophages and T cells from CD45-selected cells from tumors. Again, these antitumor effects were specific to HER2^{high} EO771/E2 tumors and were not observed in HER2^{low} EO771 tumor-bearing mice. Nevertheless, no significant difference of tumor growth was observed between mice treated with aPD1 alone or aPD1 plus BiTN_{HER}, indicating that aPD1 and BiTN_{HER} lacks the synergetic anti-tumor effect in the absence of aCD47 (FIG. 24). This result is consistent with what we found in TUBO tumors, where CD47 blockade significantly enhanced the anti-tumor effect of BiTN_{HER} alone (FIG. 20). These results further confirm that BiTN works as an adjuvant for CD47 blockade and enhance the solid tumor response to immunotherapy.

### Example 4—BiTN's Antitumor Effects Requires Host cGAS-STING Activation

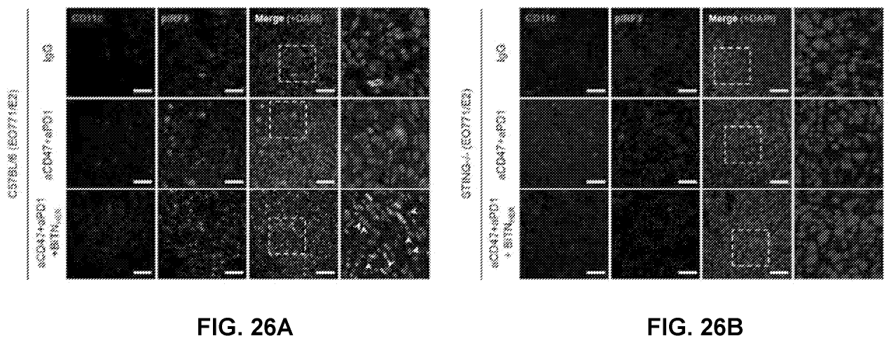

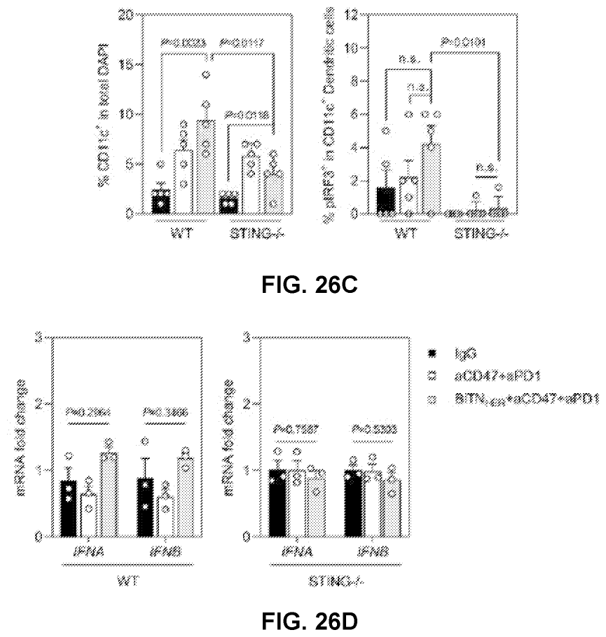

The cyclic GMP-AMP synthase-stimulator of interferon genes (cGAS-STING) pathway is involved in immune activation prompted by combined treatment with CD47 blockade and chemotherapy^{30, 31}. To test whether this innate immune sensor activation is required following combination treatment with BiTN, EO771/E2 tumors were grown in the mammary fat pad of STING knockout (KO) or wild-type (WT) mice and were treated with different regimens. Compared with the WT animals, the antitumor effect of BiTN_{HER} was completely abrogated in STING KO animals, where we found no significant difference in tumor growth after aCD47-plus-aPD1 treatment with or without BiTN_{HER} (FIG. 20B). Meanwhile, the triple combination therapy strongly induced the phosphorylation and nuclear translocation of the STING downstream transcription factor IFN regulatory factor 3 (IRF3) in the intratumoral macrophages of WT mice, but not in those of IgG-treated WT mice or STING KO mice (FIG. 20C-20E). As the major source of type I IFN production (Hopfner, K. P. & Hornung, V. Molecular mechanisms and cellular functions of cGAS402 STING signalling. Nat. Rev. Mol. Cell Biol. 21, 501-521 (2020)), STING activation led to increased IFNα/β mRNA expression in the intratumoral macrophages isolated from triple combination therapy-treated WT mice, compared with untreated or antibody-only groups (FIG. 20F). In contrast, lower IFNα/β mRNA expression was observed in tumor-associated macrophages from the triple combination-treated STING KO mice relative to those from treated WT mice. Moreover, this decreased expression seems to correlate with reduced infiltration of activated macrophages and T cells in treated tumors (FIG. 25). Since BiTN could promote macrophage phagocytosis of cancer cells, resulting in increased likelihood of activation of STING pathway by released free DNA from digested cancer cells, leading to macrophage activation and upregulation of Iba-1 expression (FIG. 16) (Martin, G. R., Blomquist, C. M., Henare, K. L. & Jirik, F. R. Stimulator of interferon genes (STING) activation exacerbates experimental colitis in mice. Sci. Rep. 9, 14281 (2019); Abdullah, A. et al. STING-mediated type-I interferons contribute to the neuroinflammatory process and detrimental effects following traumatic brain injury. J. Neuroinflammation 15, 323 (2018); Mathur, V. et al. Activation of the STING-Dependent Type I Interferon Response Reduces Microglial Reactivity and Neuroinflammation. Neuron 96, 1290-1302 e1296 (2017). Interestingly, although the total number of tumor-infiltrating DCs was elevated in WT mice, no detectable nuclear translocation of pIRF3 was observed. Expression of type I IFNs from DCs was also not significantly increased after treatment (FIG. 26), indicating that STING pathway was not activated in DCs at least 24 h after the last treatment. Thus, we speculate that only macrophages were directly engaged by the BiTN to phagocyte cancer cells. They were the first population of cells that responded to the treatment and initiated the subsequent immune responses, followed by DC recruitment and activation (Li, Z. et al. Immunogenic Cell Death Activates the Tumor Immune Microenvironment to Boost the Immunotherapy Efficiency. Adv. Sci. (Weinh), e2201734 (2022)). Collectively, these results suggest that STING activation in host cells is critical for tumor inhibitory effect of combined BiTN and immunotherapy treatment. Although CD47 and PD1 blockade had moderate antitumor effects, BiTN_{HER} was the necessary component of the combinational therapy that sensitized the tumor to these immune checkpoint inhibitors (Zitvogel, L., Galluzzi, L., Kepp, O., Smyth, M. J. & Kroemer, G. Type I interferons in anticancer immunity. Nat. Rev. Immunol. 15, 405-414 (2015); Salvagno, C. et al. Therapeutic targeting of macrophages enhances chemotherapy efficacy by unleashing type I interferon response. Nat. Cell. Biol. 21, 511-521 (2019)).

### Example 5—Combination Immunotherapy with Receptor Customized BiTN Inhibits Metastasis

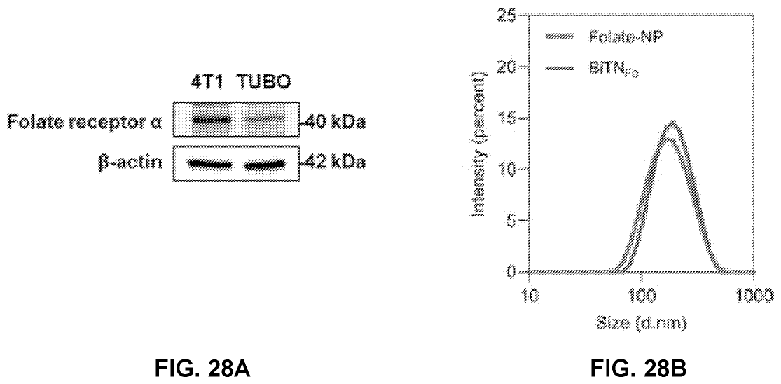

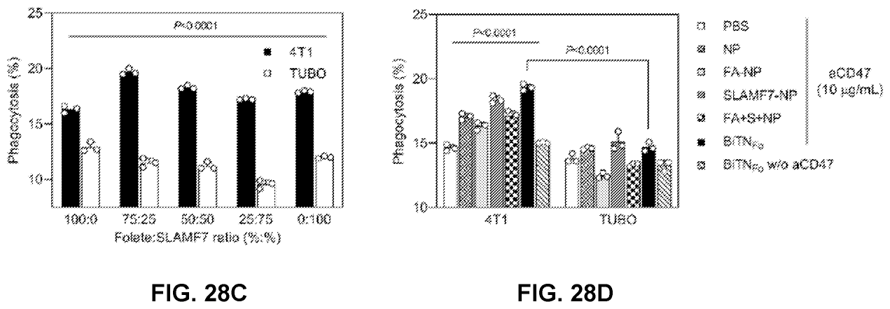

To demonstrate the versatility of BiTN platform for treating solid tumors with distinct molecular profiles, we sought to customized it to target other tumor receptors. We further developed a BiTNFo by exchanging the anti-HER2 antibody with folate to target triple-negative breast cancer (TNBC), where the folate receptor (FR) a is highly expressed (Zhang, Z. et al. Folate receptor alpha associated with triple-negative breast cancer and poor prognosis. Arch. Pathol. Lab. Med. 138, 890-895 (2014); Song, D. G. et al. Effective adoptive immunotherapy of triple-negative breast cancer by folate receptor-alpha redirected CAR T cells is influenced by surface antigen expression level. J. Hematol. Oncol. 9, 56 (2016)). BiTNFo similarly targeted and transformed FR^{high} cancer cells into SLAMF7^{high} cells. As expected, incubating 4T1 cancer cells with the optimized BiTNFo and aCD47 led to the greatest phagocytosis by macrophages relative to FR^{low} TUBO cells (FIG. 28). The increased optimal ratio of conjugated targeting ligand from 1:3 HER2:SLAMF7 on the BiTN_{HER} to 3:1 folate:SLAMF7 on the BiTNFo may be attributed to the different affinity of ligands for receptors.

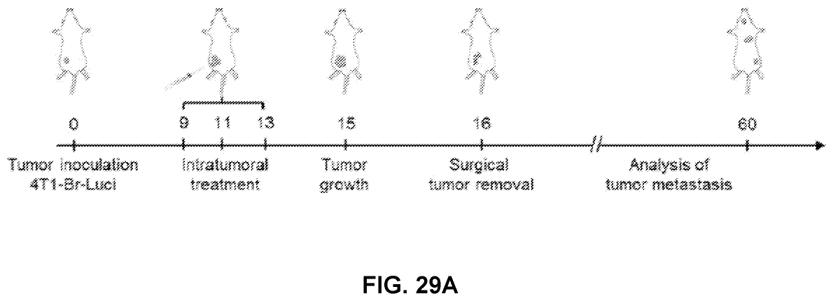

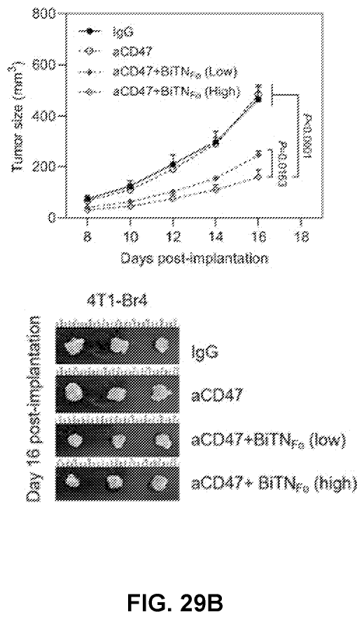

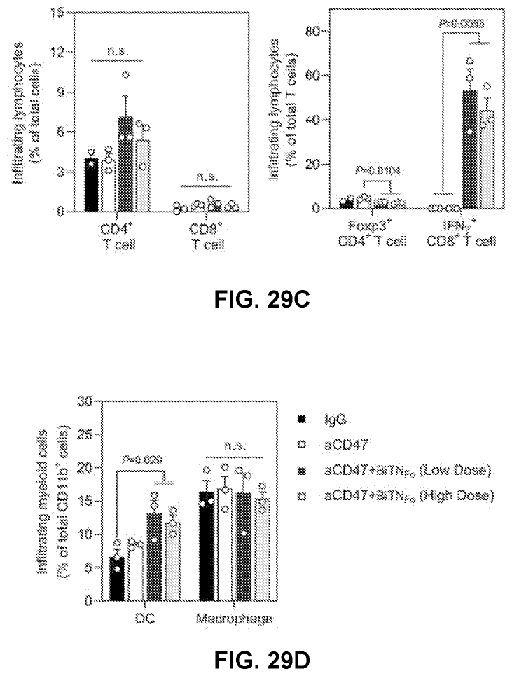

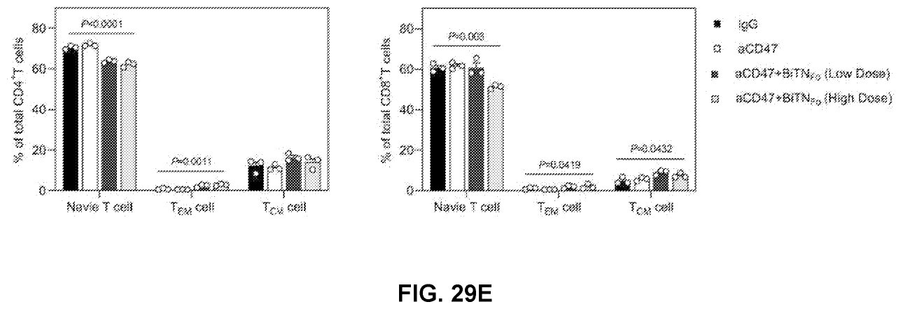

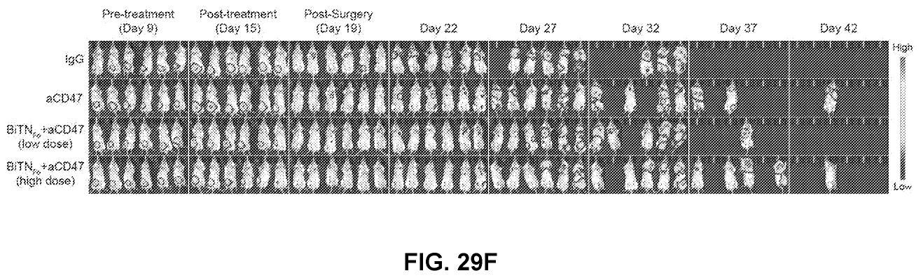

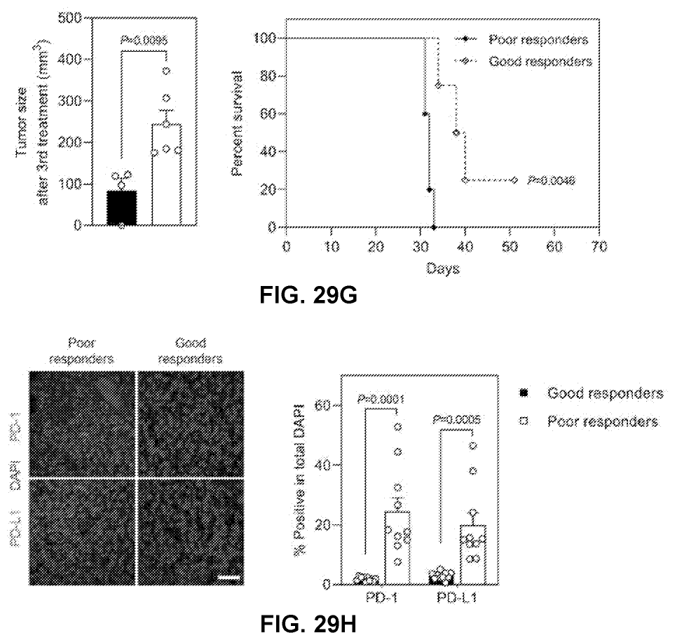

We next tested the therapeutic effect of BiTNFo by using a spontaneous 4T1 metastasis mouse model. We treated the primary tumor with intratumorally injected BiTNFo, along with intraperitoneal injections of aCD47 before surgical resection of the primary lesions. Compared with IgG- or aCD47-treated groups, the BiTNFo and aCD47 combination resulted in enhanced inhibition of local disease recurrence (FIG. 29). Evaluation of tumor infiltrating immune cells revealed increased recruitment of IFNγ⁺ cytotoxic CD8+ T cells and DCs, with elevated numbers of memory T cells in spleen (FIG. 29). However, the activated immune response in the BiTNFo-and-aCD47-treated mice did not delay distant metastasis progression or prolonged overall survival (FIG. 29). When analyzing mice that were deemed ‘good responders’ or ‘poor responders’ based on the primary tumor progression after local therapy, we found that the ‘good responders’ had significantly lower expression of PD1 and PDL1 on the tumors, which may have accounted for the increased survival rate of these mice (FIG. 29). This result may be secondary to high immune resistance through the upregulation of immune checkpoints upon adaptive immune activation (Aldea, M. et al. Overcoming Resistance to Tumor-Targeted and Immune-Targeted Therapies. Cancer Discov. 11, 874-899 (2021); Han, C. et al. Tumor cells suppress radiation-induced immunity by hijacking caspase 9 signaling. Nat. Immunol. 21, 546-554 (2020); Tumeh, P. C. et al. PD-1 blockade induces responses by inhibiting adaptive immune resistance. Nature 515, 568-571 (2014)), and therefore blockade of the PD1/PDL1 axis may enhance the therapeutic effects of BiTNFo. FIGS. 31-32 show the gating strategy used for analyzing myeloid cells, including dendritic cells (DCs), myeloid-derived suppressor cells (MDSCs), macrophages and T cells from CD45-selected cells from tumors. FIG. 34 shows the gating strategy for analyzing splenocytes from spleens.

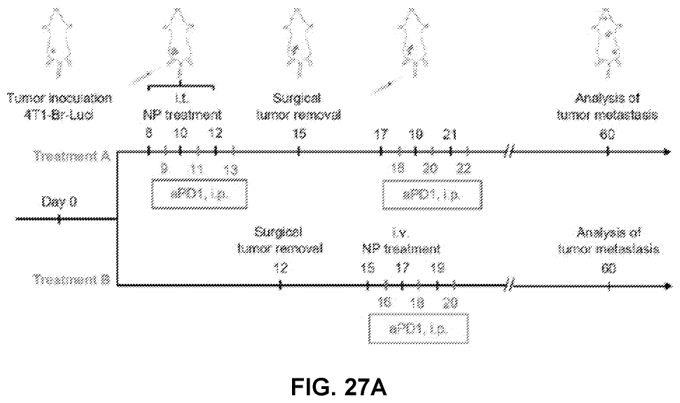

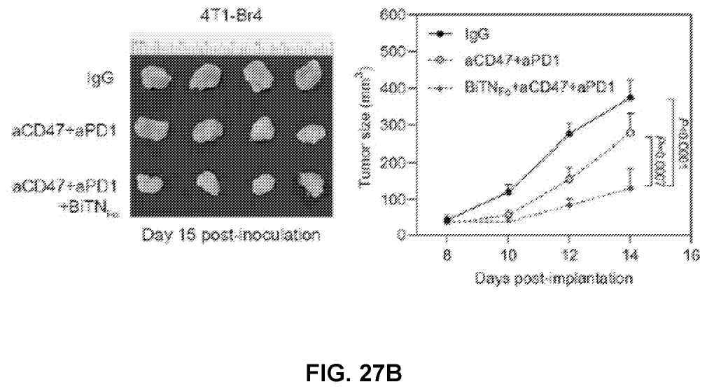

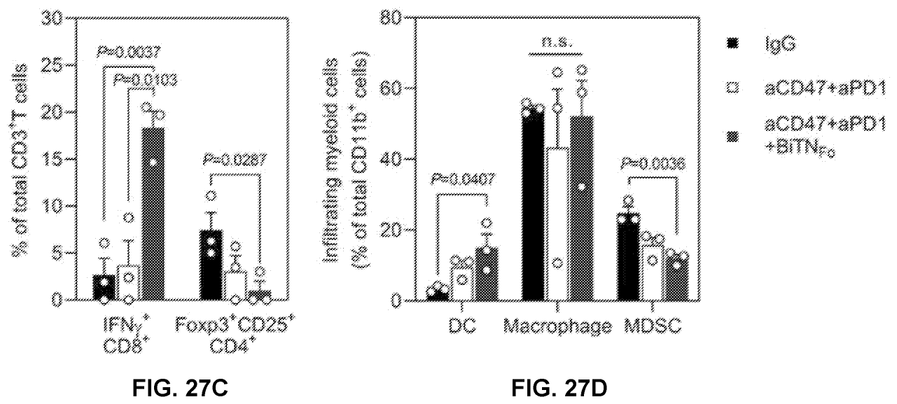

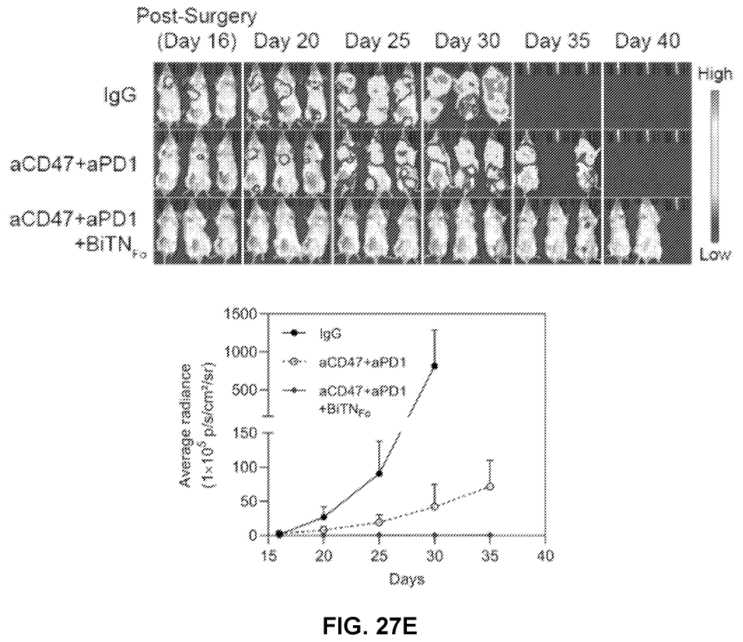

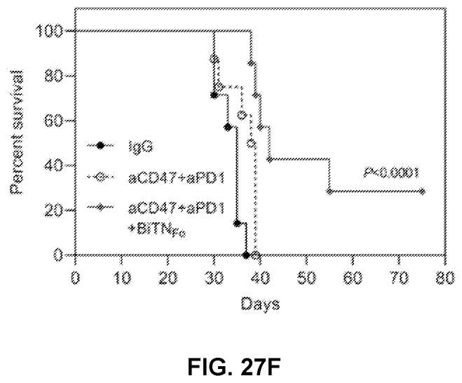

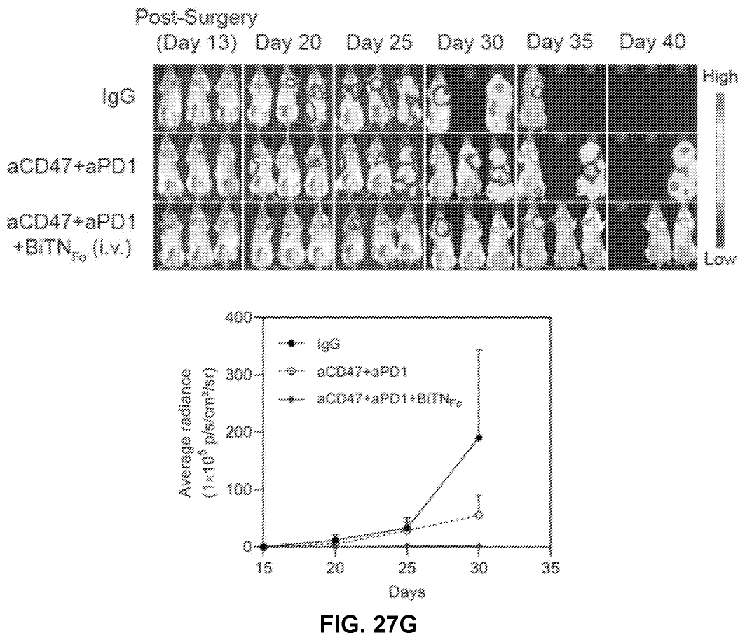

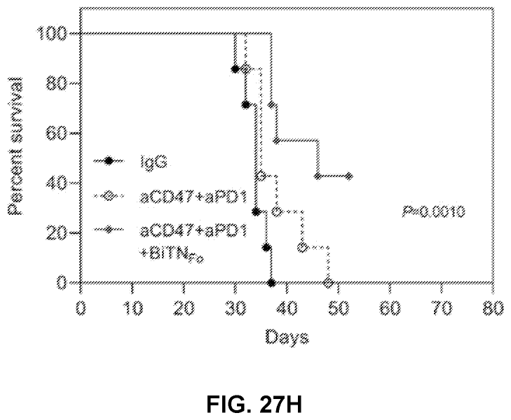

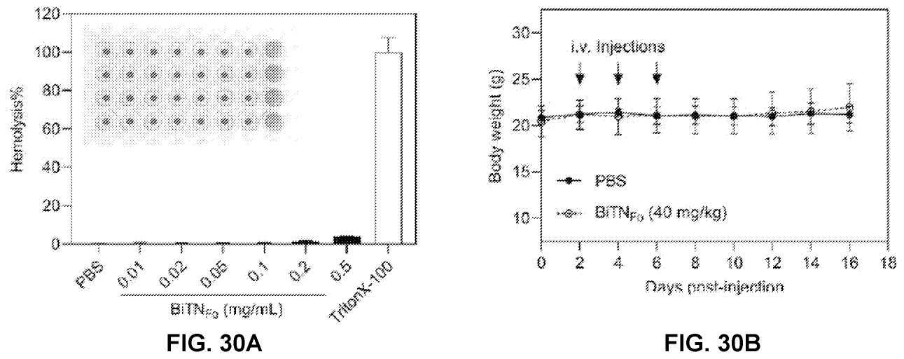

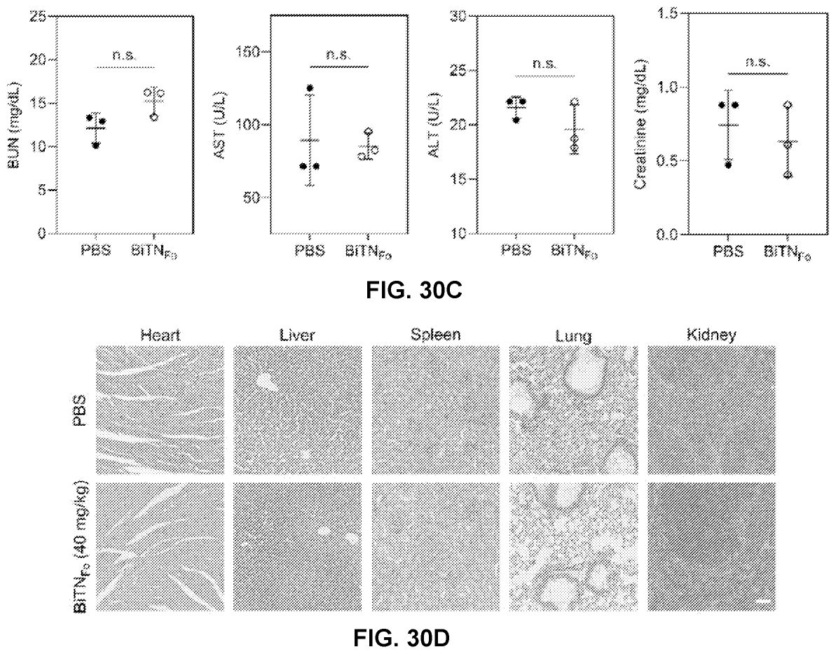

As expected, the addition of aPD1 to the treatment promoted the tumor-inhibition effects of both aCD47 alone and aCD47 plus BiTNFo on the primary tumors (FIG. 27A, 27B). In addition, compared with IgG treatment, the triple combination treatment altered the immunosuppressive tumor microenvironment, as demonstrated by increased infiltration of tumor-killing IFNγ⁺ CD8⁺ T cells and antigen-presenting DCs with corresponding decreases in tumor-promoting CD4⁺ Tregs and MDSCs (FIG. 27C, 27D). To further control the formation of tumor metastases after primary tumor removal, we tested continued treatment with BiTNFo, aCD47, and aPD1. In contrast to the previous double combination of aCD47 and BiTNFo, the triple combination treatment led to prolonged inhibition of tumor metastasis in terms of metastasis burden (size and number). Indeed, 2 of 7 mice had long term survival and remained tumor free (FIG. 27E, 27F). FIGS. 31-32 show the gating strategy used for analyzing myeloid cells, including dendritic cells (DCs), myeloid-derived suppressor cells (MDSCs), macrophages and T cells from CD45-selected cells from tumors. Finally, we did not observe notably acute toxicities from the intravenous administration of BiTNFo (FIG. 30). These results indicate that BiTN could function as a robust neoadjuvant to generate in situ tumor vaccination via promoting macrophage phagocytosis and inducing systemic inhibition of highly immunosuppressive solid tumors when combined with immune checkpoint blockade. To be noted, luciferase has been reported to be weakly immunogenic in Balb/c mice⁴⁴, which could increase the sensitivity of tumors to checkpoint blockade. However, the immunogenicity of this xeno-antigen alone was insufficient to generate potent immune response as aCD47 and aPD1 only slight prolonged mice survival. In contrast, combination of BiTN with aCD47 and aPD1 strongly boosted the immune system to eliminate residual tumors and prevent metastasis.

To further increase the translational relevance of this model, a post-surgery treatment regimen (Treatment B) was performed. In brief, 4T1-Br4-Luci cells were inoculated, and the grown primary tumors were resected on Day 12 without any pretreatment. Starting from Day 15, mice were treated by i.v. injection of BiTNFo and i.p. injection of aCD47 and aPD1 (FIG. 27A). By frequent monitoring of tumor luciferase signal, we found the similar trend of metastasis inhibition and prolonged mice survival induced by the post-surgery treatment of BiTNFo, aCD47 and aPD1 (FIG. 27G-27H). These results indicated that even without pre-generation of in-situ tumor vaccine by intratumoral treatment, i.v. injection of the BiTNFo as an adjuvant therapy was sufficient to eliminate residual tumor cells and reduce systemic diseases when combined with aCD47 and aPD1. These results further strengthened the strategy of BiTN-based immunotherapy and provided solid evidence in its antitumor efficiency in the postoperative setting.

TABLE 1
Human and mouse SLAMF7 amino acid sequences.

| | | SEQ ID |
|---|---|---|
| Name | Sequence | NO: |
| hSLAMF7₁₋₃₃₅ | MAGSPTCLTLIYILWQLTGSAASGPVKELVGSVGGAV | 3 |
| (full length) | TFPLKSKVKQVDSIVWTFNTTPLVTIQPEGGTIIVTQNR | |
| | NRERVDFPDGGYSLKLSKLKKNDSGIYYVGIYSSSLQ | |
| | QPSTQEYVLHVYEHLSKPKVTMGLQSNKNGTCVTNL | |
| | TCCMEHGEEDVIYTWKALGQAANESHNGSILPISWR | |
| | WGESDMTFICVARNPVSRNFSSPILARKLCEGAADDP | |
| | DSSMVLLCLLLVPLLLSLFVLGLFLWFLKRERQEEYIE | |
| | EKKRVDICRETPNICPHSGENTEYDTIPHTNRTILKEDP | |
| | ANTVYSTVEIPKKMENPHSLLTMPDTPRLFAYENVI | |
| hSLAMF7₂₃₋₃₃₅ | SGPVKELVGSVGGAVTFPLKSKVKQVDSIVWTFNTTP | 4 |
| (mature | LVTIQPEGGTIIVTQNRNRERVDFPDGGYSLKLSKLKK | |
| polypeptide) | NDSGIYYVGIYSSSLQQPSTQEYVLHVYEHLSKPKVT | |
| | MGLQSNKNGTCVTNLTCCMEHGEEDVIYTWKALGQ | |
| | AANESHNGSILPISWRWGESDMTFICVARNPVSRNFSS | |
| | PILARKLCEGAADDPDSSMVLLCLLLVPLLLSLFVLGL | |
| | FLWFLKRERQEEYIEEKKRVDICRETPNICPHSGENTE | |
| | YDTIPHTNRTILKEDPANTVYSTVEIPKKMENPHSLLT | |
| | MPDTPRLFAYENVI | |
| hSLAMF7₂₃₋₂₂₆ | SGPVKELVGSVGGAVTFPLKSKVKQVDSIVWTFNTTP | 5 |
| (extracellular | LVTIQPEGGTIIVTQNRNRERVDFPDGGYSLKLSKLKK | |
| domain) | NDSGIYYVGIYSSSLQQPSTQEYVLHVYEHLSKPKVT | |
| | MGLQSNKNGTCVTNLTCCMEHGEEDVIYTWKALGQ | |
| | AANESHNGSILPISWRWGESDMTFICVARNPVSRNFSS | |
| | PILARKLCEGAADDPDSSM | |
| hSLAMF7₂₃₋₁₂₄ | SGPVKELVGSVGGAVTFPLKSKVKQVDSIVWTFNTTP | 6 |
| (Ig-like V-type | LVTIQPEGGTIIVTQNRNRERVDFPDGGYSLKLSKLKK | |
| domain) | NDSGIYYVGIYSSSLQQPSTQEYVLHV | |
| hSLAMF71₃₁₋₂₀₆ | PKVTMGLQSNKNGTCVTNLTCCMEHGEEDVIYTWK | 7 |
| (Ig-like C2-type) | ALGQAANESHNGSILPISWRWGESDMTFICVAFNPVS | |
| | RNFS | |
| hSLAMF7₃₁₋₁₂₅ | GSVGGAVTFPLKSKVKQVDSIVWTFNTTPLVTIQPEG | 8 |
| (immunoglobulin- | GTIIVTQNRNRERVDFPDGGYSLKLSKLKKNDSGIYY | |
| like domain) | VGIYSSSLQQPSTQEYVLHVY | |
| hSLAMF7₂₇₋₁₀₄ | KELVGSVGGAVTFPLKSKVKQVDSIVWTFNTTPLVTI | 9 |
| (immunoglobulin- | QPEGGTIIVTQNRNRERVDFPDGGYSLKLSKLKKNDS | |
| like domain) | GIYY | |
| mSLAMF7₁₋₃₃₃ | MARFSTYIIFTSVLCQLTVTAASGTLKKVAGALDGSV | 10 |
| (full length) | TFTLNITEIKVDYVVWTFNTFFLAMVKKDGVTSQSSN | |
| | KERIVFPDGLYSMKLSQLKKNDSGAYRAEIYSTSSQA | |
| | SLIQEYVLHVYKHLSRPKVTIDRQSNKNGTCVINLTCS | |
| | TDQDGENVTYSWKAVGQGDNQFHDGATLSIAWRSG | |
| | EKDQALTCMARNPVSNSFSTPVFPQKLCEDAATDLTS | |
| | LRGILYILCFSAVLILFAVLLTIFHTTWIKKGKGCEEDK | |
| | KRVDRHQEMPDLCPHLEENADYDTIPYTEKRRPEEDA | |
| | PNTFYSTVQIPKVVKSPSSLPAKPLVPRSLSFENVI | |
| mSLAMF7₂₃₋₃₃₃ | SGTLKKVAGALDGSVTFTLNITEIKVDYVVWTFNTFF | 11 |
| (mature | LAMVKKDGVTSQSSNKERIVFPDGLYSMKLSQLKKN | |
| polypeptide) | DSGAYRAEIYSTSSQASLIQEYVLHVYKHLSRPKVTID | |
| | RQSNKNGTCVINLTCSTDQDGENVTYSWKAVGQGDN | |
| | QFHDGATLSIAWRSGEKDQALTCMARNPVSNSFSTPV | |
| | FPQKLCEDAATDLTSLRGILYILCFSAVLILFAVLLTIF | |
| | HTTWIKKGKGCEEDKKRVDRHQEMPDLCPHLEENAD | |
| | YDTIPYTEKRRPEEDAPNTFYSTVQIPKVVKSPSSLPA | |
| | KPLVPRSLSFENVI | |
| mSLAMF7₂₃₋₂₂₄ | SGTLKKVAGALDGSVTFTLNITEIKVDYVVWTFNTFF | 1 |
| (extracellular | LAMVKKDGVTSQSSNKERIVFPDGLYSMKLSQLKKN | |
| domain) | DSGAYRAEIYSTSSQASLIQEYVLHVYKHLSRPKVTID | |
| | RQSNKNGTCVINLTCSTDQDGENVTYSWKAVGQGDN | |
| | QFHDGATLSIAWRSGEKDQALTCMARNPVSNSFSTPV | |
| | FPQKLCEDAATDLTSLRG | |
| mSLAMF7₂₇₋₁₁₂ | KKVAGALDGSVTFTLNITEIKVDYVVWTFNTFFLAMV | 12 |
| (Ig-like V-type) | KKDGVTSQSSNKERIVFPDGLYSMKLSQLKKNDSGA | |
| | YRAEIYSTSSQAS | |
| mSLAMF7₁₂₈₋₂₀₃ | PKVTIDRQSNKNGTCVINLTCSTDQDGENVTYSWKA | 13 |
| (Ig-like C2-type) | VGQGDNQFHDGATLSIAWRSGEKDQALTCMARNPVS | |
| | NSFS | |
| mSLAMF7₃₁₋₁₂₂ | GALDGSVTFTLNITEIKVDYVVWTFNTFFLAMVKKDG | 14 |
| (immunoglobulin | VTSQSSNKERIVFPDGLYSMKLSQLKKNDSGAYRAEI | |
| domain) | YSTSSQASLIQEYVLHVY | |
| mSLAMF7₁₂₇₋₁₉₆ | RPKVTIDRQSNKNGTCVINLTCSTDQDGENVTYSWKA | 15 |
| (immunoglobulin | VGQGDNQFHDGATLSIAWRSGEKDQALTCMARN | |
| domain) | | |

TABLE 2
HER2 antibody Herceptin heavy and light chain sequences

| | | SEQ ID |
|---|---|---|
| Name | Sequence | NO: |
| Herceptin | EVQLVESGGGLVQPGGSLRLSCAASGFNIKDTYIHW | 2 |
| (Trastazumab) | VRQAPGKGLEWVARIYPTNGYTRYADSVKGRFTISA | |
| heavy chain | DTSKNTAYLQMNSLRAEDTAVYYCSRWGGDGFYAM | |
| | DYWGQGTLVTVSSASTKGPSVFPLAPSSKSTSGGTAA | |
| | LGCLVKDYFPEPVTVSWNSGALTSGVHTFPAVLQSS | |
| | GLYSLSSWTVPSSSLGTQTYICNVNHKPSNTKVDKKV | |
| | EPKSCDKTHTCPPCPAPELLGGPSVFLFPPKPKDTLMISR | |
| | TPEVTCWVDVSHEDPEVKFNWYVDGVEVHNAKTKPR | |
| | EEQYNSTYRWSVLTVLHQDWLNGKEYKCKVSNKALP | |
| | APIEKTISKAKGQPREPQVYTLPPSREEMTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLSL | |
| | SPG | |
| Herceptin | DIQMTQSPSSLSASVGDRVTITCRASQDVNTAVANYQQ | 16 |
| (Trastazumab) | KPGKAPKLLIYSASFLYSGVPSRFSGSRSGTDFTLTISSL | |
| light chain | QPEDFATYYCQQHYTTPPTFGQGTKVEIKRTVAAPSVF | |
| | IFPPSDEQLKSGTASWCLLNNFYPREAKVQWKVDNAL | |
| | QSGNSQESVTEQDSKDSTYSLSSTLTLSKADYEKHKVY | |
| | ACEVTHQGLSSPVTKSFNRGEC | |

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

What is claimed is:

1. A particle comprising a nanoparticle, a first molecule which binds a tumor cell, and a second molecule which allows the tumor cell to be recognized by an antigen presenting cell, wherein the first and the second molecules are linked to the nanoparticle with a molecular ratio of about 1:10 to about 10:1.

2. The particle of claim 1, wherein the first molecule and the second molecule are covalently linked to the nanoparticle.

3. The particle of claim 1 or 2, wherein the first molecule is selected from an antibody, a single-chain antibody, a nanobody, and a non-antibody antigen-binding polypeptide.

4. The particle of any one of claims 1-3, wherein the first molecule binds to a protein expressed on the tumor cell selected from the group consisting of HER2, EGFR, EGFRviii, PSMA, CEA, CA125, TROP2, CD105, GD2, GD3, VEGFR1, VEGFR2, NCAM, CD133, ADAM17, MCSP, PSCA, FOLR1, EPCAM, HER3, EGFR, PSMA, Integrin α4β1, FAP, MUC-1, P glycoprotein, AFP, ALK, CD30, ErbB3, ErbB4, Mesothelin, CAIX, CD66e, cMet, EphA2, HGF/SF, TPBG, BRCA1, BORIS, FOLR1, GAGE-1, GAGE-2, GM3, gp100, MAGE-A3, MAGE1, MAGE2, MAGE3, MAGE4, MAGE6, MAGE12, MART-1, Muc1, Muc3A, Muc3B, Muc4, Muc12, Muc13, Muc15, Muc16, Muc17, Muc21, Muc22, PRLR, or CD340.

5. The particle of any one of claims 1-4, wherein the first molecule binds to a HER2 protein or EGFR protein expressed on the tumor cell.

6. The particle of any one of claims 1-5, wherein the first molecule comprises an antibody or antigen-binding fragment that binds a HER2 or an EGFR.

7. The particle of any one of claims 1-4, wherein the first molecule is selected from the group consisting of folate, mannose, dehydroabietic acid, and any combination thereof.

8. The particle of any one of claims 1-7, wherein the second molecule is not an antibody.

9. The particle of any one of claims 1-8, wherein the second molecule comprises a signaling lymphocytic activation molecule family member 7 (SLAMF7) polypeptide or a functional fragment thereof.

10. The particle of any one of claims 1-9, wherein the second molecule comprises a full-length SLAMF7 polypeptide.

11. The particle of any one of claims 1-9, wherein the second molecule comprises an extracellular domain of SLAMF7.

12. The particle of any one of claims 1-9, wherein the second molecule comprises an Ig-like domain of SLAMF7.

13. The particle of any one of claims 1-9, wherein the second molecule comprises an Ig-like V-type domain of SLAMF7.

14. The particle of any one of claims 1-9, wherein the second molecule comprises an Ig-like C2-type domain of SLAMF7.

15. The particle of any one of claims 9-12, wherein the SLAMF7 is a human SLAMF7 polypeptide or a functional fragment thereof.

16. The particle of any one of claims 1-15, wherein the molecular ratio of the first molecule to the second molecule is about 1:3 to 3:1.

17. The particle of any one of claims 1-16, wherein the molecular ratio of the first molecule to the second molecule is about 1:3.

18. The particle of any one of claims 1-17, wherein the nanoparticle comprises a poly lactic acid polymer, a polyglycolic acid polymer, a poly(D,L-lactic-co-glycolic acid) co-polymer, a polyethylene glycol polymer, a poly(D,L-lactic-co-glycolic acid)-b-poly(ethylene glycol) copolymer with terminal methoxy groups (PLGA-b-mPEG), a poly(D,L-lactic-co-glycolic acid)-b-poly(ethylene glycol) copolymer with terminal N-hydroxysuccinimide ester groups (PLGA-b-PEG-NHS), a poly(D,L-lactic-co-glycolic acid)-b-poly(ethylene glycol) copolymer with terminal folic acid groups (PLGA-b-PEG-Folate), or any combination thereof.

19. The particle of any one of claims 1-18, wherein the nanoparticle has a size of about 70 nm to about 85 nm.

20. The particle of any one of claims 1-19, wherein the nanoparticle has a polydispersity index of about 0.09 to about 0.4.

21. The particle of any one of claims 1-20, which is stable for at least 72 hours at 37° C.

22. The particle of any one of claims 1-21, wherein the first molecule comprises a HER2 antibody or antigen-binding fragment thereof and the second molecule comprises full-length SLAMF7 polypeptide.

23. The particle of any one of claims 1-22, wherein the particle comprises about 100 to 500 first molecules (e.g., HER2 antibody or antigen-binding fragments thereof molecules) and about 500 to 1800 second molecules (e.g., full length SLAMF7 polypeptide molecules).

24. The particle of any one of claims 1-23, wherein the particle comprises about 120 to 400 first molecules (e.g., HER2 antibody or antigen-binding fragments thereof molecules) and about 500 to 1600 second molecules (e.g., full length SLAMF7 polypeptide molecules).

25. The particle of any one of claims 1-24, wherein the particle comprises about 144 and 360 HER2 antibody or antigen-binding fragments thereof molecules and about 522 and 1564 full length SLAMF7 polypeptide molecules.

26. The particle of any one of claims 1-25, wherein the particle comprises 144 HER2 antibody or antigen-binding fragments thereof molecules and 1564 full length SLAMF7 polypeptide molecules.

27. A composition comprising a particle of any one of claims 1-26 and a pharmaceutically acceptable carrier.

28. A combination therapy comprising the particle of any one of claims 1-26 or composition of claim 27 and a CD47 blocking agent.

29. The combination therapy of claim 28, wherein the CD47 blocking agent is selected from the group consisting of an anti-CD47 antibody or antigen-binding fragment thereof, an anti-CD47 nanobody, an anti-CD47 single chain antibody, a CD47 binding polypeptide, and SIRPalpha inhibitor.

30. The combination therapy of claim 28 or 29, wherein the CD47 blocking agent comprises an anti-CD47 antibody or antigen-binding fragment thereof.

31. The combination therapy of claim 28 or 29, wherein the CD47 blocking agent comprises a SIRPalpha inhibitor.

32. The combination therapy of any one of claims 28-31 further comprising an immune checkpoint inhibitor, which is not the CD47 blocking agent.

33. The combination therapy of claim 32, wherein the immune checkpoint inhibitor is selected from the group consisting of a PD-1 antibody, a fragment thereof, a PD-1 single chain antibody, a PD-1 nanobody, a PD-1 binding polypeptide, a PD-L1 antibody, a fragment thereof, a PD-L1 single chain antibody, a PD-L1 nanobody, a PD-L1 binding polypeptide, and any combination thereof.

34. The combination therapy of claim 32 or 33, wherein the immune checkpoint inhibitor is selected from a PD-1 blocking agent, a PD-L1 blocking agent, or a combination thereof.

35. A method of treating a cancer in a subject comprising administering to the subject a therapeutically effective amount of the particle of any one of claims 1-26, the composition of claim 27, or the combination therapy of any one of claims 28-34.

36. The method of claim 35, wherein the cancer is a solid tumor.

37. The method of claim 36, wherein the cancer is a breast cancer, a brain cancer, a prostate cancer, a lung cancer, a colorectal cancer, a skin cancer, a head and neck cancer, or a pancreatic cancer.

38. The method of any one of claims 35-37, wherein the particle and the CD47 blocking agent are administered at the same time or sequentially.

39. The method of any one of claims 35-37, wherein the particle, the CD47 blocking agent, and the immune checkpoint inhibitor are administered at the same time or sequentially.

40. The method of claim 38 or 39, wherein the sequential administration comprises administration of the particle, the CD47 blocking agent, and/or the immune checkpoint inhibitor consecutively or administration together in one or more compositions consecutively.

41. The method of any one of claims 35-40, wherein the particle is administered via a route selected from intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrasternal, oral, rectal, topical, epidermal, mucosal, intranasal, vaginal, rectal, sublingual administration, and combinations thereof.

42. The method of any one of claims 35-41, wherein the CD47 blocking agent is administered via a route selected from intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrasternal, oral, rectal, topical, epidermal, mucosal, intranasal, vaginal, rectal, sublingual administration, and combinations thereof.

43. The method of any one of claims 35-42, wherein the immune checkpoint inhibitor is administered via a route selected from intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural, intrasternal, oral, rectal, topical, epidermal, mucosal, intranasal, vaginal, rectal, sublingual administration, and combinations thereof.


SEQUENCE LISTING (16 sequences)

SEQ ID NO: 1 - protein, 202 aa, synthetic construct
SGTLKKVAGALDGSVTFTLNITEIKVDYVVWTFNTFFLAMVKKDGVTSQSSNKERIVFPDGLYSMKLSQLKKNDSGAYRAEIYSTSSQASLIQEYVLHVYKHLSRPKVTIDRQSNKNGTCVINLTCSTDQDGENVTYSWKAVGQGDNQFHDGATLSIAWRSGEKDQALTCMARNPVSNSFSTPVFPQKLCEDAATDLTSLRG

SEQ ID NO: 2 - protein, 446 aa, synthetic construct
  source 1..446: note = heavy chain
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSWTVPSSSLGTQTYICNVNHKPSNTKVDKKVEPKSCDKTHTCPPCPAPELLGGPSVFLFPPKPKDTLMISRTPEVTCWVDVSHEDPEVKFNWYVDGVEVHNAKTKPREEQYNSTYRWSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 335 aa, synthetic construct
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 4 - protein, 313 aa, synthetic construct
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 5 - protein, 204 aa, synthetic construct
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSM

SEQ ID NO: 6 - protein, 102 aa, synthetic construct
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHV

SEQ ID NO: 7 - protein, 76 aa, synthetic construct
PKVTMGLQSNKNGTCVTNLTCCMEHGEEDVIYTWKALGQAANESHNGSILPISWRWGESDMTFICVAFNPVSRNFS

SEQ ID NO: 8 - protein, 95 aa, synthetic construct
GSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVY

SEQ ID NO: 9 - protein, 78 aa, synthetic construct
KELVGSVGGAVTFPLKSKVKQVDSIVWTFNTTPLVTIQPEGGTIIVTQNRNRERVDFPDGGYSLKLSKLKKNDSGIYY

SEQ ID NO: 10 - protein, 333 aa, synthetic construct
MARFSTYIIFTSVLCQLTVTAASGTLKKVAGALDGSVTFTLNITEIKVDYVVWTFNTFFLAMVKKDGVTSQSSNKERIVFPDGLYSMKLSQLKKNDSGAYRAEIYSTSSQASLIQEYVLHVYKHLSRPKVTIDRQSNKNGTCVINLTCSTDQDGENVTYSWKAVGQGDNQFHDGATLSIAWRSGEKDQALTCMARNPVSNSFSTPVFPQKLCEDAATDLTSLRGILYILCFSAVLILFAVLLTIFHTTWIKKGKGCEEDKKRVDRHQEMPDLCPHLEENADYDTIPYTEKRRPEEDAPNTFYSTVQIPKVVKSPSSLPAKPLVPRSLSFENVI

SEQ ID NO: 11 - protein, 311 aa, synthetic construct
SGTLKKVAGALDGSVTFTLNITEIKVDYVVWTFNTFFLAMVKKDGVTSQSSNKERIVFPDGLYSMKLSQLKKNDSGAYRAEIYSTSSQASLIQEYVLHVYKHLSRPKVTIDRQSNKNGTCVINLTCSTDQDGENVTYSWKAVGQGDNQFHDGATLSIAWRSGEKDQALTCMARNPVSNSFSTPVFPQKLCEDAATDLTSLRGILYILCFSAVLILFAVLLTIFHTTWIKKGKGCEEDKKRVDRHQEMPDLCPHLEENADYDTIPYTEKRRPEEDAPNTFYSTVQIPKVVKSPSSLPAKPLVPRSLSFENVI

SEQ ID NO: 12 - protein, 86 aa, synthetic construct
KKVAGALDGSVTFTLNITEIKVDYVVWTFNTFFLAMVKKDGVTSQSSNKERIVFPDGLYSMKLSQLKKNDSGAYRAEIYSTSSQAS

SEQ ID NO: 13 - protein, 76 aa, synthetic construct
PKVTIDRQSNKNGTCVINLTCSTDQDGENVTYSWKAVGQGDNQFHDGATLSIAWRSGEKDQALTCMARNPVSNSFS

SEQ ID NO: 14 - protein, 92 aa, synthetic construct
GALDGSVTFTLNITEIKVDYVVWTFNTFFLAMVKKDGVTSQSSNKERIVFPDGLYSMKLSQLKKNDSGAYRAEIYSTSSQASLIQEYVLHVY

SEQ ID NO: 15 - protein, 70 aa, synthetic construct
RPKVTIDRQSNKNGTCVINLTCSTDQDGENVTYSWKAVGQGDNQFHDGATLSIAWRSGEKDQALTCMARN

SEQ ID NO: 16 - protein, 213 aa, synthetic construct
  source 1..213: note = light chain
DIQMTQSPSSLSASVGDRVTITCRASQDVNTAVANYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC